(19) United States
(12) Patent Application Publication
(10) Pub. No.: US 2025/0164473 A1
(43) Pub. Date: May 22, 2025

(54) AUTOMATED HIGH-THROUGHPUT SCREENING PLATFORM

(71) Applicant: Lawrence Livermore National Security, LLC, Livermore, CA (US)

(72) Inventors: Nicholas Noel Watkins, Livermore, CA (US); Matthew A. Coleman, Livermore, CA (US); Chao Liu, Livermore, CA (US); Steven Allen Hoang-Phou, Livermore, CA (US); Erika Jo Fong, Livermore, CA (US); Congwang Ye, Livermore, CA (US); Ted Alfred Laurence, Livermore, CA (US); William Louis Smith, Livermore, CA (US); Parker Larry Moberg, Livermore, CA (US)

(73) Assignee: Lawrence Livermore National Security, LLC, Livermore, CA (US)

(21) Appl. No.: 18/948,342

(22) Filed: Nov. 14, 2024

Related U.S. Application Data
(60) Provisional application No. 63/602,033, filed on Nov. 22, 2023.

(51) Int. Cl. G01N 33/543; G01N 33/569; G01N 35/00; G16B 40/00

(52) U.S. Cl. CPC G01N 33/54366; G01N 33/56983; G01N 35/0099; G16B 40/00

(57) ABSTRACT

Aspects of this technical solution can include transporting one or more microreactors comprising one or more polypeptides and one or more putative binding partners through a channel of an interrogation chamber, capturing data corresponding to the one or more microreactors comprising the one or more polypeptides and one or more putative binding partners in the channel, determining one or more binding affinities of the one or more polypeptides to the one or more putative binding partners based on the captured data, and generating, by a machine learning model (e.g., a neural network) receiving input based on the one or more binding affinities, output indicative of one or more predicted amino acid sequences of a polypeptide.

### CROSS-REFERENCE TO RELATED APPLICATIONS

This application claims the benefit under 35 U.S.C. § 119(e) of U.S. Provisional Patent Application No. 63/602,033, filed Nov. 22, 2023, the entire contents of which is incorporated herein by reference in its entirety.

### STATEMENT OF GOVERNMENT SUPPORT

This invention was made with government support under Grant Number PLS-21ERD039, awarded by the Laboratory Directed Research and Development (LDRD) Program, Department of Energy. The government has certain rights in the invention.

### BACKGROUND

The ability to rapidly create biological countermeasures (BCMs) to existing and/or emerging and novel biological threats, such as antibodies capable of neutralizing emerging viral variants, has continued to be a major challenge for public health for decades and, in some cases, national security. The rate at which new BCM designs can be experimentally evaluated using conventional experimental techniques is on the order of weeks and months. Thus, the use of traditional experimental techniques to evaluate, for example, polypeptide-putative binding partner binding (e.g., to screen new BCM designs), can often significantly delay the generation of suitable BCM candidates. As the need for novel and/or improved BCMs increases, for example, due to increased rates of evolution in biological systems yielding additional viral variants that can “escape” or “evade” current BCMs, the need for improved (e.g., faster) methods to assess binding between polypeptides and putative binding partners (e.g., antigens), facilitating rapid development of BCMs, are needed.

### SUMMARY

The present disclosure provides, among other things, automated, high-throughput methods of generating one or more amino acid sequences of a polypeptide and systems of automated, high-throughput screening of one or more polypeptides that bind one or more putative binding partners. Such systems and methods can, for example, increase the speed by which polypeptide-putative binding partner binding can be assessed and can incorporate such binding information to generate one or more amino acid sequences of polypeptides (e.g., with potentially improved characteristics, such as increased binding affinity to putative binding partners). Such systems and methods can fill a gap in quantitative biology, including facilitation of rapid creation of BCMs (e.g., in response to emerging viral variants). This technical solution can provide such a technical solution by combining predictive computational tools with instrumentation capabilities to produce and/or evaluate high numbers of polypeptides (e.g., polypeptide variants) for studying polypeptide-putative binding partner binding, including, for example, polypeptide-small molecule binding and polypeptide-antigen binding.

The technical solutions described herein are directed at least to a method and device for the high throughput screening of various polypeptides and putative binding partners using a novel combination of one or more of automated sample handling, micro emulsions, microfluidics manipulation, machine vision, and fluorescent interrogation. This automated system can be used to partition cell-free synthesized polypeptides into thousands of independent microreactors that are tracked and optically interrogated for assay activity, such as binding to putative binding partners in individual microreactors. Since timing of microreactor generation and position can be identified in methods of the present disclosure, an n-dimensional pooling strategy is performed to increase screening throughput compared to, for example, polypeptide-putative binding partner evaluation using conventional approaches, such as enzyme-linked immunosorbent assay (ELISA). An optical interrogation method, such as fluorescence correlation spectroscopy (FCS), can provide highly quantitative information for each “reaction” in real time (e.g., affinity constant for novel antibody binding to one or more antigen), significantly increasing the precision and quality of data. Iterative computational analysis and machine learning can optionally be combined with one or more of cell-free protein synthesis, microencapsulation and fluorescent correlation spectroscopy (FCS) to provide, for example, one or more amino acid sequences with potentially improved characteristics (e.g., increased binding affinity to putative binding partners), resulting in an automated, iterative, high-throughput screening method that allows for rapid biological countermeasure design and implementation.

At least one aspect is directed to an automated high-throughput method for obtaining one or more predicted amino acid sequences of a polypeptide. The method comprises transporting one or more microreactors comprising one or more polypeptides and one or more putative binding partners through a channel of an interrogation chamber wherein binding of the one or more polypeptides to at least one putative binding partner can occur for further detection. The method then further comprises capturing data corresponding to the bound polypeptide-binding partner complexes in the channel. The method can further comprise determining one or more binding affinities of the one or more polypeptides bound to the one or more putative binding partners based on the captured data. The method can include generating, by a machine learning model receiving input based on the one or more binding affinities, output indicative of one or more predicted amino acid sequences of a polypeptide. In one aspect, the polypeptide and/or the putative binding partner is detectably labeled, which labels may be the same or different from each other. The method can further comprise measuring, managing, and/or controlling oxygen levels within the one or more microreactors.

In some embodiments, the method further includes selecting one or more sets of microreactors in the channel of the interrogation chamber; monitor positions of the selected one or more sets of microreactors within the channel of the interrogation chamber over time. The method can include generating the output indicative of the one or more predicted amino acid sequences of the polypeptide by generating, by the machine learning model, the output indicative of the one or more predicted amino acid sequences based on the monitored positions of the selected one or more sets of microreactors.

At least one aspect is directed to a system of automated high-throughput screening of one or more polypeptides. The system comprises an interrogation chamber defining a channel having a dimension based on a size of one or more microreactors optionally comprising the one or more polypeptides and one or more putative binding partners, the channel configured to transport the one or more microreactors. The system can comprise one or more electrodes disposed at a surface of the interrogation chamber according to the dimension, the one or more electrodes to generate an electric field to hold the one or more microreactors in the channel. The system can further comprise a camera oriented toward a face of the interrogation chamber, the camera to capture data corresponding to the one or more microreactors in the channel. The system can further comprise a memory and one or more processors configured to determine, based on the captured data, one or more binding affinities of a polypeptide with a putative binding partner, and generate, by a machine learning model receiving input based on the one or more binding affinities, output indicative of one or more predicted amino acid sequences of a polypeptide. In some embodiments, oxygen levels within the system can be measured, managed, and/or controlled.

At least one aspect is directed to a method of automated high-throughput screening of polypeptides that can be obtained or generated from cell-free protein synthesis. The method can include, for example, encapsulating, into one or more microcapsules in a carrier fluid, one or more sample fluids with one or more corresponding encapsulating fluids comprising one or more polypeptide and one or more putative binding partner to the one or more polypeptide. In one aspect, the one or more polypeptide and/or the one or more putative binding partners are detectably labeled. The method can comprise transporting the microcapsules into a channel of an interrogation chamber. The method can further comprise capturing one or more images of the microcapsules in the channel. The method can further comprise determining, based on the one or more images, one or more binding affinities of the one or more polypeptides with one or more putative binding partners in the carrier fluid. The method can further comprise generating, by a machine learning model receiving input based on the one or more binding affinities, one or more predicted amino acid sequences of one or more polypeptides. The method can further comprise an iterative process of the method of automated high-throughput screening of the one or predicted amino acid sequences of the one or more polypeptide to further refine the polypeptides by evaluating the one or more predicted amino acids sequence against the same or different putative binding partners, or alternatively evaluating the one or more putative amino acid sequences of the one or more polypeptide by a biological assay, e.g., ELISA.

At least one aspect is directed to a system of automated high-throughput screening of polypeptide sequences that can be present in biological samples. The system can comprise an interrogation chamber defining a channel having a dimension based on a size of one or more microcapsules, the channel configured to transport the microcapsules. The system can comprise one or more electrodes disposed at an exterior surface of the interrogation chamber according to the dimension, the one or more electrodes to generate an electric field to hold the microcapsules in the channel. The system can comprise a camera oriented toward a face of the interrogation chamber, the camera to capture one or more images of the microcapsules in the channel. The system can further comprise a memory and one or more processors configured to determine, based on the one or more images, one or more binding affinities of a first analyte in the sample fluids with a second analyte in the carrier fluid, and generate, by a machine learning model receiving input based on the one or more binding affinities, one or more predicted amino acid sequences.

### BRIEF DESCRIPTION OF THE FIGURES

These and other aspects and features of the present implementations are depicted by way of example in the figures discussed herein. Present implementations can be directed to, but are not limited to, examples depicted in the figures discussed herein. Thus, this disclosure is not limited to any figure or portion thereof depicted or referenced herein, or any aspect described herein with respect to any figures depicted or referenced herein.

FIG. 1 depicts an example system, according to this disclosure.

FIG. 2 depicts an example apparatus, according to this disclosure.

FIG. 3A depicts an example encapsulation of cell-free lysates, according to this disclosure.

FIG. 3B depicts an example encapsulated cell-free lysates, according to this disclosure.

FIG. 4 depicts example candidate samples, according to this disclosure.

FIG. 5 depicts an example controller architecture, according to this disclosure.

FIG. 6 depicts an exploded view of an interrogation chamber, according to this disclosure.

FIG. 7 depicts an interrogation chamber with microcapsules, according to this disclosure.

FIG. 8 depicts a plan view of microcapsules in an interrogation chamber, according to this disclosure.

FIG. 9 depicts a plan view of tracked movement of microcapsules in an interrogation chamber, according to this disclosure.

FIG. 10A depicts screening instrumentation, according to this disclosure.

FIG. 10B depicts screening instrumentation, according to this disclosure.

FIG. 10C depicts a fluorescence signal from microcapsules, according to this disclosure.

FIG. 10D depicts correlation curves from microcapsules, according to this disclosure.

FIG. 11A depicts protein produced inside microcapsules, according to this disclosure.

FIG. 11B depicts correlation curves from microcapsules, according to this disclosure.

FIG. 11C depicts amplitudes of correlation curves, according to this disclosure.

FIG. 11D depicts diffusion times from microcapsules, according to this disclosure.

FIG. 12A depicts protein produced inside microcapsules, according to this disclosure.

FIG. 12B depicts correlation curves from microcapsules, according to this disclosure.

FIG. 12C depicts amplitudes of correlation curves, according to this disclosure.

FIG. 12D depicts a concentration curve from GFP, according to this disclosure.

FIG. 12E depicts a correlation to time delay, according to this disclosure.

FIG. 12F depicts diffusion times from GFP, according to this disclosure.

FIG. 13 depicts an example method of automated high-throughput screening, according to this disclosure.

FIG. 14 depicts an example method of automated high-throughput screening, according to this disclosure.

FIGS. 15A-15C is an example to measure binding of a polypeptide during cell-free synthesis to a putative binding partner by fluorescence correlation spectroscopy (FCS). FIG. 15A shows a schematic of reactions comprising the cell-free expression lysate, the protein of interest (“putative binding partner” or “binding partner”), and a plasmid encoding a fluorescently labeled polypeptide (“polypeptide-GFP” or “Protein2-GFP”) that is hypothesized to bind to the putative binding partner. Reaction solutions (˜5 μL) are incubated in sample wells between coverslips, keeping a layer of air above. Different sample wells may contain different variants of polypeptide-GFP when screening the polypeptide for binding, for example. The concentration of polypeptide-GFP increases over time as the expression proceeds while that of the putative binding partner remains constant. FIG. 15B shows samples can be measured over time by simple one-color FCS. A 488 nm laser excites the GFP, and emitted photons are selected by a confocal pinhole (PH) and detected by two avalanche photodiodes (APDs). OJB: objective, DM: dichroic mirror, L: lens, BS: beamsplitter, BP: bandpass filter. FIG. 15C Bottom: data consists of arrival times of each photon on the detectors, collected for different samples over several hours of polypeptide synthesis. Top left: calculated correlations are expected to shift down and left over experimental time, as amplitude decreases due to increasing polypeptide concentration while overall diffusion time decreases due to an excess of free polypeptide-GFP over its putative binding partner. Top right: a two-species fit to correlations yields the fraction of polypeptide-GFP bound and its concentration, producing a binding curve from which the binding affinity (KD) is determined.

FIGS. 16A-16E demonstrates that FCS tracks fluorescent polypeptides synthesized in cell-free lysates in real time. FIG. 16A provides a schematic of production of GFP by cell-free reaction containing a GFP plasmid (pGFP) that was measured by FCS over time. FIGS. 16B-16C shows measurements of a commercially available (Rabbit Biotech) (FIG. 16B) and homemade (FIG. 16C) cell-free lysates without plasmid. Left: correlation curves (left) are colored black (earlier time points) to white (later) and show noise from the lysate background. Right: photon counts for both lysates initially decreased before increasing, and the homemade lysate had lower lysate background signal. FIGS. 16D-16E shows measurements of GFP production over time in commercial (FIG. 16D) and homemade (FIG. 16E) lysates containing 1 μg/ml (0.23 nM) plasmid encoding GFP at room temperature. From left to right: correlation curves, both raw and normalized, colored light (earlier) to dark (later) green; photon count rate; fitted amplitude f₀ and diffusion times Tpover experimental time. Concentrations (green) were calculated from the f₀ given the calibrated detection volume and were corrected for background signal using the lysate-only data at every time point. The initially lower f₀ and t_{D} are due to background noise contribution at low GFP concentrations.

FIGS. 17A-17B demonstrate rate and amount of polypeptide synthesis by CFPS can be tuned by plasmid concentration. FIGS. 17A-17B show photon count rate (top), fitted amplitude f₀ (middle) and diffusion times tp (bottom) are plotted over experimental time for several GFP plasmid concentrations in commercial (FIG. 17A) and homemade (FIG. 17B) cell-free lysates. Lysate-only photon count rates are plotted as gray open circles. Calculated concentrations included background subtraction. The homemade lysate had slightly slower production rates than the commercial lysate but similar final yields.

FIGS. 18A-18H shows binding affinity between GFP and an anti-GFP antibody in buffer solution. FIG. 18A shows a schematic that to measure the binding affinity between GFP and an anti-GFP antibody, samples containing a constant concentration of GFP (200 pM) and varying concentrations of the unlabeled antibody in buffer were measured by FCS. FIG. 18B shows normalized correlation curves shifted right at higher antibody concentrations. Inset shows non-normalized correlations. FIG. 18C shows diffusion times from one-species fits to correlations increased as a function of antibody concentration. FIG. 18D shows the fraction of GFP bound to antibody was calculated from two-species fits to correlations. The quadratic binding equation was fitted to the binding data to obtain the dissociation constant K_{D}=0.03+−0.01 nM and antibody concentration P=0.19+−0.03 nM, which agrees with the known amount of GFP used. FIG. 18E shows a schematic that binding curve was also obtained by varying the GFP concentration while keeping constant the unlabeled antibody concentration (400 pM). FIG. 18F shows normalized correlation curves shifted left at higher GFP concentrations. Inset shows non-normalized curves with lower amplitudes at higher GFP concentrations. FIG. 18G shows diffusion times from one-species fits decreased as a function of GFP concentration. FIG. 18H shows the fraction of GFP bound calculated from two-species fits to correlations, plotted against GFP concentration. The variant quadratic binding equation was fitted to the measurements to obtain K_{D}=0.02+−0.01 nM and antibody concentration P=0.41+−0.02 nM, consistent with both the K_{D} determined by the standard binding method (FIGS. 18A-18D) and the known amount of antibody used. This antibody and GFP have very tight binding with low picomolar K_{D}.

FIGS. 19A-19F shows binding affinity between GFP and an anti-GFP antibody in cell-free lysates. Control experiments as described in FIGS. 18A-18H, varying either unlabeled antibody (19A-19C) or GFP (19D-19F), were repeated in cell-free lysates. Higher amounts of the non-varying protein were required than in the buffer experiments in order to measure GFP signals above the higher background due to lysate. FIG. 19A shows a schematic that to measure the binding affinity between GFP and an anti-GFP antibody, samples containing a constant concentration of GFP and varying concentrations of the unlabeled antibody in cell-free lysate were measured by FCS. FIG. 19B shows vary antibody in commercial lysate, using 2 nM GFP. Fitted K_{D}=0.04+−0.02 nM, GFP concentration P=1.9+−0.1 nM. FIG. 19C shows vary antibody in homemade lysate, using 5 nM GFP. Fitted K_{D}=0.25+−0.17 nM, GFP concentration P=5.3 +-0.6 nM. FIG. 19D shows a schematic that binding curve was also obtained by varying the GFP concentration while keeping constant the unlabeled antibody concentration. FIG. 19E shows vary GFP in commercial lysate, using 2 nM antibody. Fitted K_{D}=0.06+−0.02 nM, antibody concentration P=2.2+−0.1 nM. FIG. 19F shows fary GFP in homemade lysate, using 10 nM antibody. Fitted K_{D}=0.28+−0.1 nM, antibody concentration P=8.1+−0.4 nM.

FIGS. 20A-20G shows determination of binding affinity from FCS measurements of polypeptide synthesis over time in cell-free lysates. FIG. 20A shows a schematic of samples containing GFP plasmid and antibody in cell-free lysate were measured over time by FCS for production of GFP and antibody binding. FIG. 20B-20D show results from 5 μg/ml (1.2 nM) plasmid and 20 nM antibody in commercial lysate. Normalized correlation curves (FIG. 20B) shifted left as GFP concentration increased over time (FIG. 20B inset). Total GFP concentrations were determined from the total amplitude f₀ of two-species fits and background subtracted using lysate-only data at each time point (FIG. 20C). Fraction of GFP bound calculated from two-species fits is plotted against the measured GFP concentrations (FIG. 20D). The variant quadratic binding equation was fitted to the measurements to obtain K_{D}=11+−1.5 nM and antibody concentration P=19+−1.5 nM. FIG. 20E-20G show correlation curves (FIG. 20E), total amplitudes and background-subtracted concentrations (FIG. 20F), and binding curve (FIG. 20G) for measurements of 2 μg/ml (0.5 nM) plasmid and 20 nM antibody in homemade lysate. Note that the amplitude was initially low (FIG. 20F) due to the dominant contribution of lysate background to the total signal, as also evident in the earliest correlations (FIG. 20E). Fitted K_{D}=15+−3 nM, antibody concentration P=20+−2 nM. The fitted antibody concentrations are consistent with the known amount (20 nM) used. Since the concentration of antibody, required to be sufficiently high to measure signal above lysate background, was much higher than the true low-picomolar K_{D} (FIGS. 18A-18H and FIGS. 19A-19F), these real-time binding curves establish an upper bound on the KD.

FIGS. 21A-21C demonstrate use of higher concentrations of trace putative binding partner establishes upper bound on the K_{D} measured by cell free protein synthesis. FIG. 21A shows a compilation of the measured K_{D} vs. the “trace” protein concentration P used from all binding experiments. Accurate determination of K_{D} typically requires P<K_{D}, a condition often not met in biological experiments due to practical constraints. Control experiments, in which sub-nanomolar P (antibody or GFP concentration) were used (solid markers) (from FIGS. 18A-18H and FIGS. 19A-19F), suggested that the true affinity was ˜30 pM or less (shaded area). Much higher K_{D}S were measured from real-time protein synthesis experiments (open markers) due to a combination of the much higher P (antibody concentration) required in lysate background, GFP maturation time, and/or nonequilibrium binding. Slowing down and lowering protein expression produced K_{D} measurements more consistent with control experiments (red open markers) (FIGS. 21B, 21C). FIG. 21B shows binding experiment from protein synthesis using 0.5 μg/ml (0.12 nM) GFP plasmid and 20 nM antibody in commercial lysate. Left: total GFP concentrations were determined from the total amplitude f₀ of two-species fits and background subtracted using lysate-only data at each time point. Right: fraction of GFP bound calculated from two-species fits is plotted against the measured GFP concentrations. The variant quadratic binding equation was fitted to the measurements to obtain K_{D}=0.2+−0.1 nM and antibody concentration P=18.7+−0.5 nM (red open circle in (a)). FIG. 21C shows total amplitudes and background-subtracted concentrations (left), and binding curve (right) for measurements of 0.5 μg/ml (0.12 nM) plasmid and 20 nM antibody in homemade lysate. Fitted K_{D}=0.18+−0.06 nM, antibody concentration P=9.3+−0.2 nM (red open triangle in (a)).

FIGS. 22A-22E shows determination of binding affinity from protein synthesis inside microcapsules. FIG. 22A Top: schematic of double emulsion droplet generation on a microfluidic device. Through the flow of fluids in two round capillaries with tapered openings within a larger square capillary, the core fluid (cell-free reaction) (yellow) is packaged into droplets with a polymer shell (red) surrounded by the carrier fluid (light blue). Droplets pass under a UV lamp to crosslink the shell polymer before collection and measurement of the microcapsules by FCS. Bottom: image of droplet formation in the region of the interface between the three fluids. FIGS. 22B-22E show FCS measurements of protein synthesis and binding inside a microcapsule containing 1.45 μg/ml (0.34 nM) GFP plasmid and 20 nM antibody in commercial lysate. FIG. 22B shows normalized correlation curves shifted left as GFP concentration increased over time (inset). FIG. 22C shows total GFP concentrations were determined from the total amplitude f₀ of two-species fits and background subtracted using lysate-only data at each time point. FIG. 22D shows diffusion times from one-species fits decreased over time as GFP concentration increased. FIG. 22E shows the fraction of GFP bound calculated from two-species fits is plotted against the measured GFP concentrations. The variant quadratic binding equation was fitted to the measurements to obtain K_{D}=0.11+−0.08 nM and antibody concentration P=22.2+−0.5 nM.

FIG. 23 provides a schematic overview of technologies of the present disclosure.

FIG. 24 provides a schematic of an interrogation chamber of the present disclosure comprising electrodes integrated into the chamber to create a DEP exit gate or “parking lot” that can hold microreactors within the chamber while they are being loaded or undergoing analysis.

FIG. 25 shows images of an interrogation chamber (top) loaded with microdroplets comprising cell-free lysates comprising DNA templates encoding mCherry and GFP and microscopy images of such microdroplets (bottom).

FIG. 26 shows images of an interrogation chamber (top) loaded with microdroplets comprising cell-free lysates comprising DNA templates encoding mCherry and GFP and microscopy images of such microdroplets (bottom).

FIG. 27 shows images of an interrogation chamber (top) loaded with microdroplets comprising cell-free lysates comprising a DNA template encoding mCherry and microscopy images of such microdroplets (bottom).

FIG. 28 shows images of an interrogation chamber (top) loaded with microdroplets comprising cell-free lysates comprising a DNA template encoding mCherry and microscopy images of such microdroplets (bottom).

FIG. 29 shows images of an interrogation chamber (top) loaded with microdroplets comprising cell-free lysates comprising a DNA template encoding mCherry and microscopy images of such microdroplets (bottom).

FIG. 30 shows images of an interrogation chamber (top) loaded with microdroplets comprising cell-free lysates comprising a DNA template encoding mCherry and microscopy images of such microdroplets (bottom).

FIG. 31 shows images of an interrogation chamber (top) loaded with microdroplets comprising cell-free lysates comprising a DNA template encoding mCherry and microscopy images of such microdroplets (bottom).

FIG. 32 shows images of an interrogation chamber (top) loaded with microdroplets comprising cell-free lysates comprising a DNA template encoding mCherry and microscopy images of such microdroplets (bottom).

FIG. 33 shows images of an interrogation chamber (top) loaded with microdroplets comprising cell-free lysates comprising a DNA template encoding mCherry and microscopy images of such microdroplets (bottom).

FIG. 34 shows images of an interrogation chamber (top) loaded with microdroplets comprising cell-free lysates comprising a DNA template encoding GFP and microscopy images of such microdroplets (bottom).

FIG. 35 shows images of an interrogation chamber (top) loaded with microdroplets comprising cell-free lysates comprising a DNA template encoding GFP and microscopy images of such microdroplets (bottom).

FIG. 36 shows images of an interrogation chamber (top) loaded with microdroplets comprising cell-free lysates comprising a DNA template encoding GFP and microscopy images of such microdroplets (bottom).

FIG. 37 shows images of an interrogation chamber (top) loaded with microdroplets comprising cell-free lysates comprising a DNA template encoding GFP and microscopy images of such microdroplets (bottom).

FIG. 38 shows images of an interrogation chamber (top) loaded with microdroplets comprising cell-free lysates comprising a DNA template encoding GFP and microscopy images of such microdroplets (bottom).

FIG. 39 shows images of an interrogation chamber (top) loaded with microdroplets comprising cell-free lysates comprising a DNA template encoding mCherry and microscopy images of such microdroplets (bottom).

FIG. 40 shows a schematic of a “parking lot” of the present disclosure.

FIG. 41 provides FCS results of GFP expression from CFPS in microdroplets conducted by loading microdroplets into “parking lots” with concentrations 0 μg/mL of plasmid encoding GFP.

FIG. 42 provides FCS results of GFP expression from CFPS in microdroplets conducted by loading microdroplets into “parking lots” with concentrations 1 μg/mL of plasmid encoding GFP.

FIG. 43 provides FCS results of GFP expression from CFPS in microdroplets conducted by loading microdroplets into “parking lots” with concentrations 5 μg/mL of plasmid encoding GFP.

FIG. 44 provides FCS results of GFP expression from CFPS in microdroplets conducted by loading microdroplets into “parking lots” with concentrations 15 μg/mL of plasmid encoding GFP.

FIG. 45 depicts an example method of automated high-throughput screening, according to this disclosure.

FIG. 46 provides a schematic of partial loading (left) and a fluid robot, which can be utilized to complete such partial loading.

FIG. 47 shows FCS counts of GFP produced in microdroplets of different concentrations of GFP plasmid.

FIG. 48 shows the distribution of microdroplets of differing samples and darker “spacer” microdroplets in a system according to the present disclosure.

FIG. 49 shows an example of monitoring particles or microdroplets in an interrogation chamber according to the present disclosure.

### DETAILED DESCRIPTION

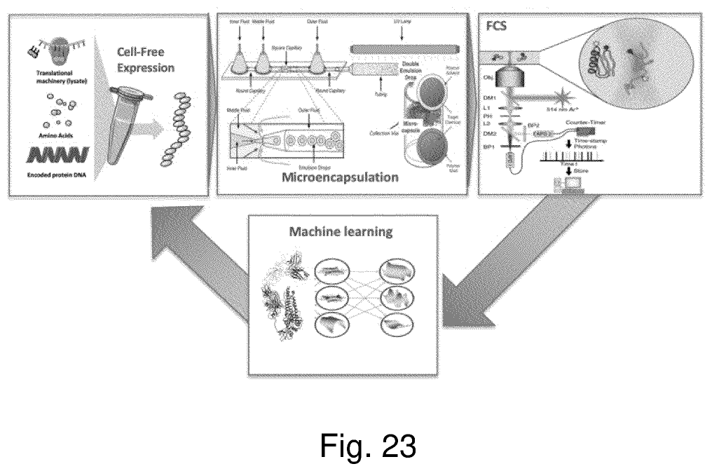

This disclosure is directed to, among other things, methods and devices for high-throughput production and/or assessment of polypeptides (e.g., antibodies and antibody fragments), including their potential binding to putative binding partners (e.g., epitopes and antigens). Such approaches utilize a novel combination of one or more of automated sample handling, microreactors, microfluidics manipulation, machine vision, and optical interrogation (FIG. 23). In some embodiments, approaches involve in silico iterative design that can rapidly generate thousands of predicted amino acid sequences of interest for production as functional polypeptides and proteins which can then be studied for their effects on function or binding to putative binding partners. Binding information can then be incorporated into a machine learning model which can then generate one or more predicted amino acid sequences of a polypeptide, e.g., that may demonstrate improved characteristics, such as enhanced binding affinity to putative binding partners. For example, methods of the present disclosure can involve evaluation of antibodies (Ab) for antigen binding, including, for example, binding specificity. Existing methods used for characterizing Ab-antigen (Ag) binding, rely heavily on techniques such as surface plasmon resonance (SPR) or enzymatic assays like ELISA, which often suffer from low throughput, linear scalability, and high requirements for purity and yield. Beyond those requirements, typical workflows involve the use of mammalian cells to express the protein of interest for purification and usage in downstream assays, which has an associated time-cost of weeks to months prior to developing, optimizing and running assays.

The technical solutions in this disclosure remove at least these key limitations and enables simultaneous screening and kinetic characterization of polypeptides and putative binding partners in relatively shorter timescales than traditional experimental methods (e.g., in 1- to 3-days). Technologies of the present disclosure can combine cell-free protein synthesis (CFPS), high-throughput microfluidics, and fluorescence correlation spectroscopy (FCS) (or any permutation thereof) to rapidly produce polypeptides (i.e., antibodies) in the presence of putative binding partners (e.g., antigens) within thousands of microreactors and measure polypeptide production as well as their binding kinetics to putative binding partners in real time. Without wishing to be bound by any one theory, it is understood that the flexibility of this system stems from, at least in part, the use of CFPS, where any compatible polynucleotide can be converted into polypeptide and/or putative binding partner within hours, bypassing the need for mammalian cell-based expression. Non-linear scaling can be achieved through microfluidic encapsulation of the starting polypeptides (e.g., polypeptides with known amino acid sequences) to generate independent discrete partitions containing polypeptide and/or putative binding partner-producing cell-free protein synthesis reactions. Readouts from optical interrogation methods, such as FCS, enable rapid evaluation of any nascent polypeptide-putative binding partner complexes forming within the microreactors, enabling assessment of binding interactions in real-time, the data from which can then be incorporated into the in silico iterative design to produce one or more additional predicted amino acid sequences (e.g., with improved binding characteristics). Since this platform can utilize, for example, fluorescent signal detection for interrogating pico- to nanoliter scale microreactor volumes, reagent usage can be minimized while still maximizing signal to noise.

Aspects of this technical solution are described herein with reference to the figures, which are illustrative examples of this technical solution. The figures and examples below are not meant to limit the scope of this technical solution to the present implementations or to a single implementation, and other implementations in accordance with present implementations are possible, for example, by way of interchange of some or all of the described or illustrated elements. Where certain elements of the present implementations can be partially or fully implemented using known components, only those portions of such known components that are necessary for an understanding of the present implementations are described, and detailed descriptions of other portions of such known components are omitted to not obscure the present implementations. Terms in the specification and claims are to be ascribed no uncommon or special meaning unless explicitly set forth herein. Further, this technical solution and the present implementations encompass present and future known equivalents to the known components referred to herein by way of description, illustration, or example.

### Definitions

Unless defined otherwise, all technical and scientific terms used herein generally have the same meaning as commonly understood by one of ordinary skill in the art to which this disclosure belongs. All nucleotide sequences provided herein are presented in the 5′ to 3′ direction. Although any methods and materials similar or equivalent to those described herein can be used in the practice or testing of the present disclosure, particular, non-limiting exemplary methods, devices, and materials are now described. All technical and patent publications cited herein are incorporated herein by reference in their entirety. Nothing herein is to be construed as an admission that the disclosure is not entitled to antedate such disclosure by virtue of prior disclosure.

It is also to be understood that the terminology used herein is for the purpose of describing particular embodiments only, and is not intended to be limiting.

The practice of the present disclosure will employ, unless otherwise indicated, conventional techniques of tissue culture, immunology, molecular biology, microbiology, cell biology and recombinant DNA, which are within the skill of the art. See e.g., Green and Sambrook eds. (2012) Molecular Cloning: A Laboratory Manual, 4^{th} edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6^{th} edition; Gait ed. (1984) Oligonucleotide Synthesis; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi ed. (2007) In Vitro Transcription and Translation Protocols, 2^{nd} edition; Guisan ed. (2006) Immobilization of Enzymes and Cells; Perbal (1988) A Practical Guide to Molecular Cloning, 2^{nd} edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4^{th} edition; and Herzenberg et al. eds (1996) Weir's Handbook of Experimental Immunology, 5^{th} edition.

As used in this specification and the appended claims, the singular forms “a”, “an” and “the” include plural references unless the context clearly dictates otherwise. For example, reference to “a cell” includes a combination of two or more cells, and mixtures thereof.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2% or alternatively 1%. It is to be understood, although not always explicitly stated, that all numerical designations are preceded by the term “about”. It also is to be understood, although not always explicitly stated, that the reagents described herein are merely exemplary and that equivalents of such are known in the art.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) (±) 15%, 10%, 5%, 2%, or 1%. Preferably ±5%, more preferably ±1%, and still more preferably +0.1% from the specified value, as such variations are appropriate to perform the disclosed methods.

The term “antigen” as used herein is defined as a molecule that can provoke an immune response and comprises one or more epitopes. In one aspect, the “antigen” is a naturally occurring or synthetically that comprises, or consists essentially of, or yet further consists of one or more putative epitopes and in one aspect, is a fragment of the antigen. When the antigen provokes an immune response, the immune response may involve either antibody production, or the activation of specific immunologically-competent cells, or both. The skilled artisan will understand that any macromolecule, including proteins, polypeptides, small molecule, or polynucleotide can serve as an antigen. Furthermore, antigens can be derived from recombinant or genomic DNA. A skilled artisan will understand that any DNA, which comprises a nucleotide sequence or a partial nucleotide sequence encoding a polypeptide or protein that in one aspect elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to elicit the desired immune response.

As used herein, the term “antibody” refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, variable domain fragment (Fv), Fab and F(ab)2, as well as single chain antibodies (scFv), humanized antibodies, fully human antibodies, antibody variants, synthetic antibodies, and modified or “derivatized” antibodies. In some embodiments, antibody refers to such assemblies (e.g., intact antibody molecules, immunoadhesins, or variants thereof) which have significant known specific immunoreactive activity to an antigen of interest (e.g., a “putative binding partner”). Antibodies and immunoglobulins comprise light and heavy chains, with or without an interchain covalent linkage between them. Basic immunoglobulin structures in vertebrate systems are relatively well understood.

In some embodiments, an antibody is a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules. In one embodiment, the antibody or antibody molecule comprises, e.g., consists of, an antibody fragment.

In some embodiments, an antibody is a “modified” antibody or antibody fragment or a “derivative” of an antibody or an antibody fragment. The term “derivative” refers to an antibody or antibody fragment that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example, antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc.

In some embodiments, the altered carbohydrate modifications modulate one or more of the following: solubilization of the antibody or antibody fragment, facilitation of subcellular transport and secretion of the antibody or antibody fragment, promotion of antibody or antibody fragment assembly, conformational integrity, and antibody- or antibody fragment-mediated effector function. In a specific embodiment, the altered carbohydrate modifications enhance antibody-or antibody fragment-mediated effector function relative to the antibody or antibody fragment lacking the carbohydrate modification. Carbohydrate modifications that lead to altered antibody-or antibody fragment-mediated effector function are well known in the art. See e.g., Shields et al., J. Biol. Chem. 277 (30): 26733-26740 (2002); Davies J. et al., Biotechnology & Bioengineering 74(4): 288-294 (2001). Methods of altering carbohydrate contents are also known to those skilled in the art. See, e.g., Wallick et al. J. Exp. Med. 168(3):1099-1109 (1988); Tao et al. J. Immunol. 143(8): 2595-2601 (1989); Routledge et al., Transplantation 60(8): 847-53 (1995); Elliott et al., Nature Biotechnol. 21:414-21 (2003); Shield et al. J. Biol. Chem. 277(30): 26733-26740 (2002).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody or antibody fragment derivative will possess a similar or identical function as the parental antibody or antibody fragment. In another embodiment, an antibody or antibody fragment derivative will exhibit an altered activity relative to the parental antibody or antibody fragment. For example, a derivative antibody (or antibody fragment) can bind to its epitope more tightly or be more resistant to proteolysis than the parental antibody (or antibody fragment).

The term “antibody fragment” refers to a portion of an intact antibody and refers to the antigenic determining variable regions of an intact antibody. In some embodiments, the term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, Fv fragments, scFv antibody fragments, linear antibodies, single domain antibodies such as sdAb (either V_{L} or V_{H}), camelid VHH domains, and multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3)(see U.S. Pat. No. 6,703,199, which describes fibronectin polypeptide minibodies). “Fab” means a monovalent antigen-binding fragment of an immunoglobulin that is composed of the light chain and part of the heavy chain. F(ab′)2 means a bivalent antigen-binding fragment of an immunoglobulin that contains both light chains and part of both heavy chains.

As used herein, the term “Fv fragment” or “variable domain fragment” refers to a VH domain and a VL domain of an antibody specifically binding to an antigen, both domains forming together a Fv fragment. In some embodiment, Fv fragments means an antibody fragment comprising the V_{H} and V_{L} domains of an antibody, wherein these domains are present in a single polypeptide chain. Generally, the Fv fragment polypeptide further comprises a polypeptide linker between the V_{H} and V_{L} domains polypeptide that enables the scFv to form.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

As used herein, the term “antibody heavy chain” refers to the larger of the two types of polypeptide chains present in all antibody molecules in their naturally occurring conformations.

As used herein, the term “antibody light chain” refers to the smaller of the two types of polypeptide chains present in all antibody molecules in their naturally occurring conformations. Kappa (κ) and lambda (λ) light chains refer to the two major antibody light chain isotypes.

As used herein, the term “synthetic antibody” means an antibody, which is generated using recombinant DNA technology, such as, for example, an antibody expressed by a bacteriophage. The term should also be construed to mean an antibody, which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “antibody variant” includes synthetic and engineered forms of antibodies which are altered such that they are not naturally occurring, e.g., antibodies that comprise at least two heavy chain portions but not two complete heavy chains (such as, domain deleted antibodies or minibodies); multi-specific forms of antibodies (e.g., bi-specific, tri-specific, etc.) altered to bind to two or more different antigens or to different epitopes on a single antigen); heavy chain molecules joined to scFv molecules and the like. In addition, the term “antibody variant” includes multivalent forms of antibodies (e.g., trivalent, tetravalent, etc., antibodies that bind to three, four or more copies of the same antigen.

As used herein, the term “complementarity determining region” or “CDR” refers to the sequences of amino acids within antibody variable regions which confer antigen specificity and binding affinity. For example, in general, there are three CDRs in each heavy chain variable region (e.g., CDRH1, CDRH2, and CDRH3) and three CDRs in each light chain variable region (CDRL1, CDRL2, and CDRL3). The precise amino acid sequence boundaries of a given CDR can be determined using any of a number of well-known schemes, including those described by Kabat et al. (1991), “Sequences of Proteins of Immunological Interest,” 5^{th} Ed. Public Health Service, National Institutes of Health, Bethesda, Md. (“Kabat” numbering scheme), Al-Lazikani et al., (1997) JMB 273,927-948 (“Chothia” numbering scheme), or a combination thereof, North et al., J. Molecular Biology, 406 (2): 228-256 (2011) and; Lefranc et al. Nucl. Acids Res. 27:209-212 (1999) or Ruiz et al. Nucl. Acids Res. 28:219-221 (2000)) (IMGT numbering scheme).

Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). Under the IMGT numbering scheme, in some embodiments, the CDR amino acids in the VH are numbered 27-38 (HCDR1), 56-65 (HCDR2), and 105-120 (HCDR3); and the CDR amino acid residues in the VL are numbered 27-38 (LCDR1), 56-65 (LCDR2), and 105-120 (LCDR3).

In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

As used herein, the term “humanized antibodies” refers to human forms of non-human (e.g., murine) antibodies, and are chimeric immunoglobulins, immunoglobulin chains or fragments thereof (such as Fv, Fab, Fab′, F(ab′)2 or other antigen-binding subsequences of antibodies), which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues.

Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature 321:522-525 (1986); Reichmann et al., Nature 332:323-329 (1988); Presta, Curr. Op. Struct. Biol. 2:593-596 (1992).

As used herein, the term “fully human antibody” refers to an immunoglobulin, such as an antibody, where the whole molecule is of human origin or consists of an amino acid sequence identical to a human form of the antibody. In some embodiments, the antibodies disclosed herein are fully human antibodies. In some embodiments, the antibodies are humanized antibodies.

As used herein, the term “immunoglobulin” or “Ig,” defines a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

As used herein the term, “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

As used herein, the term “single chain antibodies” refer to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird, Science 242:423-442 (1988); Huston et al., Proc. Natl. Acad. Sci. USA 85:5879-5883 (1988); Ward et al., Nature 334:54454 (1989); Skerra et al., Science 242:1038-1041 (1988).

As used herein, the terms “cell-free protein synthesis” or “synthesized in a cell-free lysate” or “cell-free protein expression” refer to a method of producing recombinant polypeptides in solution using biomolecular translation machinery extracted from a range of different cell types (e.g., Escherichia coli), but without the use of living cells. Cell-free protein synthesis methods require a polynucleotide template (e.g., plasmid, PCR product) encoding the polypeptide to be synthesized and a solution comprising all the necessary components to drive transcription and/or translation. In some embodiments, such a solution is a “cell-free extract” or “cell-free lysate”, which comprises cytosolic and organelle material, and does not comprise the cell membranes of the cell from which the cell-free extract was produced. Cell-free protein synthesis methods are described in the art and their use is well within the level of a person of ordinary skill in the art. In some embodiments, cell-free protein synthesis is utilized to produce polypeptides of the disclosure (e.g., in a microreactor). In some embodiments, cell-free protein synthesis is utilized to produce putative binding partners of the disclosure (e.g., in a microreactor). cell-free protein synthesis is utilized to produce polypeptides and putative binding partners of the disclosure (e.g., in a microreactor).

As used herein, the term “conservative sequence modifications” is intended to refer to amino acid modifications that do not significantly affect or alter the binding characteristics of a polypeptide (e.g., an antibody) containing the amino acid sequence. Such conservative modifications include amino acid substitutions, additions and deletions. Modifications can be introduced into a polypeptide by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, for example, one or more amino acid residues within the CDR regions of an antibody can be replaced with other amino acid residues from the same side chain family and the altered antibody can be tested for the ability to bind antigens using the assays described herein.

A “composition” is intended to mean a combination of active agent and another compound or composition, inert (for example, a liquid carrier, pharmaceutically acceptable carrier, detectable agent or label) or active, such as an adjuvant.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives (e.g., sodium benzoate, potassium sorbate, and methyl hydroxybenzoate), and the like. “Consisting of” shall mean excluding more than trace elements of other ingredients and substantial method steps for administering the compositions disclosed herein. Embodiments defined by each of these transition terms are within the scope of this disclosure.

As used herein, the term “encoding” refers to the inherent property of specific sequences of nucleotides in a polynucleotide (such as a gene, a cDNA, or an mRNA), to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system (e.g., a cell-free protein synthesis system). Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the term “host cell” refers to a cell into which exogenous polypeptide (recombinant or otherwise) or virus has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous polynucleotides (e.g., a recombinant nucleic acid sequence) and/or for introduced viruses to replicate. Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO K1, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, and a cell line derived from an aforementioned cell. In some embodiments, the cell comprises one or more viral genes.

A host cell, according to the present disclosure, may be, but is not limited to, prokaryotic cells, eukaryotic cells, archeobacteria, bacterial cells, insect cells, yeast, mammal cells, and/or plant cells. Bacteria envisioned as host cells can be either gram-negative or gram-positive, e.g. Escherichia coli, Erwinia sp., Klebsellia sp., Lactobacillus sp. or Bacillus subtilis. In some embodiments, the host cell is a yeast cell. In that embodiment, the yeast host cell is selected from the group consisting of Saccharomyces cerevisiae, Hansenula polymorpha, and Pichia pastoris.

As used herein, “homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure. In some embodiments, “homology” or “identity” or “similarity” can also refer to two nucleic acid molecules that hybridize under stringent conditions.

As used herein, the term “immune response” as used herein is defined as a cellular response to an antigen that occurs when lymphocytes identify antigenic molecules as foreign and induce the formation of antibodies and/or activate lymphocytes to remove the antigen.

As used herein, the term “label” intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to a polynucleotide that will provide a signal upon expression of the inserted sequences, such as green fluorescent protein (GFP) and the like. The term also includes purification tags or labels that aid in the isolation of biological materials from mixed populations. While the term “label” generally intends compositions covalently attached to the composition to be detected, in one aspect it specifically excludes naturally occurring nucleosides and amino acids that are known to fluoresce under certain conditions (e.g., temperature, pH, etc.) when positioned within the polynucleotide or protein in its native environment and generally any natural fluorescence that may be present in the composition to be detected. The label may be detectable by itself (e.g., radioisotope labels or fluorescent labels) or, in the case of an enzymatic label, may catalyze chemical alteration of a substrate compound or composition that is detectable. The labels can be suitable for small-scale detection or more suitable for high-throughput screening. As such, suitable labels include, but are not limited to magnetically active isotopes, non-radioactive isotopes, radioisotopes, fluorochromes, chemiluminescent compounds, dyes (e.g., Cyanine-5 (Cy5)), tandem dyes (also called a Fluorescence Resonance Energy Transfer (FRET) pair or dye), and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6^{th} ed). Examples of luminescent probes include, but are not limited to, aequorin and luciferases.

Examples of suitable fluorescent labels include, but are not limited to, fluorescein, fluorescein isothiocyanate (FITC), green fluorescent protein (GFP), mCherry, Cy5, tetramethylrhodamine (TRITC), red fluorescent protein (RFP), rhodamine, tetramethylrhodamine, eosin, erythrosin, coumarin, methyl-coumarins, pyrene, Malacite green, stilbene, Lucifer Yellow, CASCADE BLUE™, and Texas Red.

As used herein, the term “flexible polypeptide linker” or “linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser) n, where n is a positive integer equal to or greater than 1. For example, n=1, n=2, n=3. N=4, n=5 and n=6, n=7, n=8, n=9 and n=10. In one embodiment, the flexible polypeptide linkers include, but are not limited to, (Gly4 Ser)4 or (Gly4 Ser)3. In another embodiment, the linkers include multiple repeats of (Gly2Ser), (GlySer) or (Gly3Ser).

As used herein, a “model” generally comprises an approach to generating and analyzing particular data using particular techniques to obtain a particular desired result, and may employ various supervised and unsupervised machine learning and artificial intelligence techniques, statistical analyses, etc. The data may comprise specific data in particular formats and structures, and application of the model may include implementing a specific combination of techniques and analyses to obtain, for example, a particular solution, and/or a classifier capable of receiving certain inputs and providing certain outputs, etc. The model may be multi-stage, such that first-stage input data may be generated and processed to obtain a first-stage result that in turn is used to generate second-stage input data (or the first-stage result may itself be the second-stage input data) that is analyzed to obtain a second-stage result, which may itself be a final desired result or may be subjected to further analysis or processing to obtain the final desired result. Datasets may include, for example, training datasets used to train classifiers, and other datasets that may serve as inputs to trained classifiers to obtain desired outputs.

As used herein, the term “microdroplets” refers to discrete volumes of aqueous sample (e.g., a sample fluid comprising one or more polypeptides) in water-immiscible encapsulation fluids, thereby forming aqueous sample droplets in water-immiscible encapsulation fluids (water-in-oil emulsions). As used herein, “water-immiscible” refers to a liquid that does not mix with water, creating an interface between the liquid and water, e.g., mineral oil, fluorinated oil, paraffin oil, etc. In some embodiments, a microdroplet of the present disclosure has a diameter of from about 10 μm to about 1 mm, about 10 μm to about 900 μm, about 10 μm to about 800 μm, about 10 μm to about 700 μm, about 10 μm to about 600 μm, about 10 μm to about 500 μm, about 10 μm to about 400 μm, about 10 μm to about 300 μm, or about 10 μm to about 200 μm. In some embodiments, a microdroplet of the present disclosure has a diameter of about 10 μm, about 25 μm, about 50 μm, about 100 μm, about 200 μm, about 300 μm, about 400 μm, about 500 μm, about 600 μm, about 700 μm, about 800 μm, about 900 μm, or about 1 mm. In a preferred embodiment, a microdroplet of the present disclosure has a diameter of about 300 μm. Such “microdroplets” can be further encapsulated in an aqueous sample fluid (water-in-oil-in-water double emulsions) to form a “microcapsule”. In some embodiments, a microcapsule of the present disclosure has a diameter of from about 50 μm to about 600 μm, about 100 μm to about 600 μm, about 200 μm to about 600 μm, about 300 μm to about 600 μm, about 400 μm to about 600 μm, about 50 μm to about 500 μm, about 100 μm to about 500 μm, about 200 μm to about 500 μm, about 300 μm to about 500 μm, or about 400 μm to 500 μm. In some embodiments, a microcapsule of the present disclosure has a diameter of about 50 μm, about 100 μm, about 200 μm, about 300 μm, about 400 μm, about 500 μm, or about 600 μm. In a preferred embodiment, a microcapsule of the present disclosure has a diameter of about 400 μm to 500 μm. Throughout the present disclosure, “microdroplets” and “microcarriers” can be referred to collectively as “microreactors.” In some embodiments, “microreactors” comprise more than one “core” (e.g., “multi-core capsules”). Methods for producing microreactors described herein and are known in the art and selecting and using such methods to produce microreactors in accordance with the technologies described herein is well within the level of one of ordinary skill in the art.

As used herein, the term “modified” means a changed state or structure of a molecule or cell of the invention. Molecules may be modified in many ways, including chemically, structurally, and functionally. Cells may be modified through the introduction of nucleic acids.

As used herein, the term “neutralization” refers to the ability of an antibody by itself to inhibit infection of susceptible cells or, in the case of some extracellular organisms, to inhibit an initial pathogenic step. In some embodiments, neutralization of infectivity in vitro means that antibodies are capable of blocking the infectivity or pathogenesis of viruses, bacteria, parasites, and fungi. Neutralization generally occurs as a result of interfering with an organism's attachment to host tissues. Several mechanisms account for the neutralization of a given organism. In addition, a single antibody or antibodies with different specificities can neutralize a given organism, at least in vitro, through multiple mechanisms.

The term “protein,” “peptide,” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide or polypeptide must contain at least two amino acids and no limitation is placed on the maximum number of amino acids which may comprise a protein's or peptide's or polypeptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or a gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment disclosed herein that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, fragment, polypeptide or nucleic acid, intends those having minimal homology while still maintaining desired structure or functionality. Unless specifically recited herein, it is contemplated that any polynucleotide, polypeptide or protein mentioned herein also includes equivalents thereof. In one aspect, an equivalent polynucleotide is one that hybridizes under stringent conditions to the polynucleotide or complement of the polynucleotide as described herein for use in the described methods. In another aspect, an equivalent antibody or antigen binding polypeptide or Fab (fragment antigen binding) intends one that binds with at least 70%, or alternatively at least 75%, or alternatively at least 80%, or alternatively at least 85%, or alternatively at least 90%, or alternatively at least 95% affinity or higher affinity to a reference antibody or antigen binding fragment. In another aspect, the equivalent thereof competes with the binding of the antibody or antigen binding fragment to its antigen under a competitive ELISA assay. In another aspect, an equivalent intends at least about 80% homology or identity and alternatively, at least about 85%, or alternatively at least about 90%, or alternatively at least about 95%, or alternatively 98% percent homology or identity and exhibits substantially equivalent biological activity to the reference protein, polypeptide or nucleic acid.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters. In particular, exemplary programs include BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+Spupdate+PIR. Details of these programs can be found at the following Internet address: ncbi.nlm.nih.gov/cgi-bin/BLAST. Sequence identity and percent identity can determined by incorporating them into clustalW (available at the web address: genome.jp/tools/clustalw/, last accessed on Jan. 13, 2017).

As used herein, a “processor” or “processing unit” may comprise a single processor, which can have one or more cores, or multiple processors. Processors can include one or more general-purpose primary processor as well as one or more special-purpose co-processors such as graphics processors, digital signal processors, or the like. Some or all processors may be implemented using customized circuits, such as application specific integrated circuits (ASICs) or field programmable gate arrays (FPGAs). In some embodiments, such integrated circuits execute instructions that are stored on the circuit itself. In other embodiments, processors can execute instructions stored in local or remote computing storage devices.

As used herein, the term “putative binding partner” refers to any molecule (e.g., polypeptide, small molecule) that may or does specifically bind to a particular polypeptide. The putative binding partner may be a single molecule or a combination of molecules, such as a complex of polypeptides. For example, a “putative binding partner” may be an amino acid molecule, a polypeptide, a polynucleotide, a small molecule, or a combination thereof. In some embodiments, a “putative binding partner” is or comprises an antigen.

As used herein, the term “recognize or bind” intends that the binding agent, e.g., a polypeptide, a antibody, antigen binding fragment or a Fab (fragment antigen binding) is more likely than not to bind to its intended target or binding partner.

As used herein, the term “sequence identity” refers to the subunit sequence identity between two polymeric molecules particularly between two amino acid molecules, such as, between two polypeptide molecules. When two amino acid sequences have the same residues at the same positions, then they are identical at that position. For example, if a position in each of two polypeptide molecules is occupied by an Arginine, then the two polypeptides are identical. The identity or extent to which two amino acid sequences have the same residues at the same positions in an alignment is often expressed as a percentage. The identity between two amino acid sequences is a direct function of the number of matching or identical positions. For example, if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, a “subject” of diagnosis or treatment is a cell or an animal such as a mammal, or a human. A subject is not limited to a specific species and includes non-human animals subject to diagnosis or treatment and are those subject to infections or animal models, for example, simians, murines, such as, rats, mice, chinchilla, canine, such as dogs, leporids, such as rabbits, livestock, sport animals, and pets. Human patients are included within the term as well.

As used herein, “small molecule” is broadly used to refer to an organic, inorganic, or organometallic compound typically having a molecular weight of less than about 5 kDa.

As used herein, the term “specificity” refers to the ability to specifically bind (e.g., immunoreact with) a given target antigen (e.g., a human target antigen). For example, a polypeptide (e.g., an antibody) may be monospecific and contain one or more binding sites, which specifically bind a target (e.g., a putative binding partner) or a polypeptide (e.g., an antibody) may be multi-specific and contain two or more binding sites which specifically bind the same or different targets (e.g., putative binding partners). In certain embodiments, a polypeptide (e.g., an antibody) is specific for two different (e.g., non-overlapping) portions of the same target (e.g., putative binding partner). In certain embodiments, a polypeptide (e.g., an antibody) is specific for more than one target (e.g., putative binding partner).

As used herein, the term “specifically binds,” with respect to a polypeptide (e.g., an antibody), means a polypeptide or binding fragment thereof (e.g., Fv fragment or scFv) which recognizes a specific antigen, but does not substantially recognize or bind other molecules (e.g., other molecules in a sample). For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, a chimeric antigen receptor, or a peptide with a second chemical species (e.g., a putative binding partner), to mean that the interaction is dependent upon the presence of a particular structure (e.g., an antigenic determinant or epitope) on the chemical species; for example, a chimeric antigen receptor recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

As used herein, a “storage medium” can include any combination of volatile storage media (e.g., dynamic random-access memory (DRAM), static random-access memory (SRAM), synchronous DRAM (SDRAM), or the like) and/or non-volatile storage media (e.g., magnetic or optical disk, flash memory, or the like). Storage media can be fixed, removable, or upgradeable as desired. Storage media can be physically or logically divided into various subunits such as a system memory, a read-only memory (ROM), and a permanent storage device. The memory of a system can be a read-and-write memory device or a volatile read-and-write memory, such as dynamic random-access memory. The system memory can store some or all of the instructions and data that a system's processors need at runtime. The ROM can store static data and instructions that are needed by the processors. The permanent storage device can be a non-volatile read-and-write memory device that can store instructions and data even when the memory device is powered down. The term “non-transitory storage medium” as used herein includes any medium in which data can be stored indefinitely (subject to overwriting, electrical disturbance, power loss, or the like) and does not include carrier waves and transitory electronic signals propagating wirelessly or over wired connections. Local storage media may be intended to provide working memory for processors, providing fast access to programs and/or data to be processed while reducing traffic on the network. Storage for larger quantities of data can be provided on the network by one or more mass storage subsystems that can be interconnected to one or more computing devices. Mass storage subsystem can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystems. In some embodiments, additional data storage resources may be accessible via the network (potentially with increased latency).

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing units to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing units can provide various functionality for server system and client computing system, including any of the functionality described herein as being performed by a server or client, or other functionality associated with message management services.

As used herein, a “system” means one or more computing devices capable of receiving data inputs, executing computing code, and generating data outputs, and may be part of a network of co-located and/or remote computing devices capable of transmitting and receiving data via wired or wireless communication protocols. The system may include a server system that can operate in response to requests received via a network interface that can connect multiple server systems or other computing devices to each other, providing scalable systems capable of managing high volumes of activity. Techniques for managing server systems and server farms (collections of server systems that cooperate) can be used, including dynamic resource allocation and reallocation. Network interfaces can provide a connection to the network, such as a wide area network (e.g., the Internet) to which a network interface of server system 1100 is also connected. Network interfaces can include a wired interface (e.g., Ethernet) and/or a wireless interface implementing various RF data communication standards such as Wi-Fi, Bluetooth, or cellular data network standards (e.g., 3G, 4G, LTE, 5G, etc.). Server systems can interact with various user-owned or user-operated devices via a wide-area network such as the Internet. Client computing systems can be implemented, for example, as a consumer device such as a smartphone, other mobile phone, tablet computer, wearable computing device (e.g., smart watch, eyeglasses), desktop computer, laptop computer, and so on. Client computing systems can communicate via network interfaces and can include computer components such as processors, storage media, network interfaces, user input devices, and user output devices. Suitable devices can be selected based on the demands to be placed on client computing system; for example, client computing system can be implemented as a “thin” client with limited processing capability or as a high-powered computing device. Client computing system can be provisioned with program code executable by processors to enable various interactions with server system of a message management service such as accessing messages, performing actions on messages, and other interactions described herein. Some client computing systems can also interact with a messaging service independently of the message management service.

“Liposomes” are microscopic vesicles consisting of concentric lipid bilayers. A liposome is an example of a carrier, e.g., a pharmaceutically acceptable carrier. Structurally, liposomes range in size and shape from long tubes to spheres, with dimensions from a few hundred Angstroms to fractions of a millimeter. Vesicle-forming lipids are selected to achieve a specified degree of fluidity or rigidity of the final complex providing the lipid composition of the outer layer. These are neutral (cholesterol) or bipolar and include phospholipids, such as phosphatidylcholine (PC), phosphatidylethanolamine (PE), phosphatidylinositol (PI), and sphingomyelin (SM) and other types of bipolar lipids including but not limited to dioleoylphosphatidylethanolamine (DOPE), with a hydrocarbon chain length in the range of 14-22, and saturated or with one or more double C═C bonds. Examples of lipids capable of producing a stable liposome, alone, or in combination with other lipid components are phospholipids, such as hydrogenated soy phosphatidylcholine (HSPC), lecithin, phosphatidylethanolamine, lysolecithin, lysophosphatidylethanol-amine, phosphatidylserine, phosphatidylinositol, sphingomyelin, cephalin, cardiolipin, phosphatidic acid, cerebrosides, distearoylphosphatidylethan-olamine (DSPE), dioleoylphosphatidylcholine (DOPC), dipalmitoylphosphatidylcholine (DPPC), palmitoyloteoylphosphatidylcholine (POPC), palmitoyloleoylphosphatidylethanolamine (POPE) and dioleoylphosphatidylethanolamine 4-(N-maleimido-triethyl) cyclohexane-1-carboxylate (DOPE-mal). Additional non-phosphorous containing lipids that can become incorporated into liposomes include stearylamine, dodecylamine, hexadecylamine, isopropyl myristate, triethanolamine-lauryl sulfate, alkyl-aryl sulfate, acetyl palmitate, glycerol ricinoleate, hexadecyl stereate, amphoteric acrylic polymers, polyethyloxylated fatty acid amides, and the cationic lipids mentioned above (DDAB, DODAC, DMRIE, DMTAP, DOGS, DOTAP (DOTMA), DOSPA, DPTAP, DSTAP, DC-Chol). Negatively charged lipids include phosphatidic acid (PA), dipalmitoylphosphatidylglycerol (DPPG), dioteoylphosphatidylglycerol and (DOPG), dicetylphosphate that are able to form vesicles. Typically, liposomes can be divided into three categories based on their overall size and the nature of the lamellar structure. The three classifications, as developed by the New York Academy Sciences Meeting, “Liposomes and Their Use in Biology and Medicine,” December 1977, are multi-lamellar vesicles (MLVs), small uni-lamellar vesicles (SUVs) and large uni-lamellar vesicles (LUVs). The biological active agents can be encapsulated in such for administration in accordance with the methods described herein.

A “micelle” is an aggregate of surfactant molecules dispersed in a liquid colloid. A typical micelle in aqueous solution forms an aggregate with the hydrophilic “head” regions in contact with surrounding solvent, sequestering the hydrophobic tail regions in the micelle center. This type of micelle is known as a normal phase micelle (oil-in-water micelle). Inverse micelles have the head groups at the center with the tails extending out (water-in-oil micelle). Micelles can be used to attach a polynucleotide, polypeptide, antibody, antigen binding fragment, vaccine, or composition described herein to facilitate efficient delivery to the target cell or tissue.

### Modes For Carrying Out The Disclosure

This disclosure is directed to, among other things, methods and devices for high-throughput production and/or assessment of polypeptides and their potential binding to putative binding partners. Such methods utilize a novel combination of one or more of automated sample handling, microreactors, microfluidics manipulation, machine vision, and optical interrogation. In some embodiments, such approaches involve in silico iterative design that can rapidly generate thousands of predicted amino acid sequences of interest which can then be produced as polypeptides and proteins and their function and/or binding (e.g., binding affinity) to putative binding partners can be evaluated. Subsequently, the in silico iterative design process can generate one or more predicted amino acid sequences by a machine learning model (e.g., a neural network, a random forest, a support vector machine, a transformer model, a large language model, etc.) receiving input based on the one or more binding affinities. In some embodiments, the one or more predicted amino acid sequences generated by the machine learning model have improved characteristics compared to the initially generated amino acid sequences by the in silico iterative design process or polypeptides with known amino acid sequences (e.g., can bind to a certain putative binding partner, e.g., increased binding affinity to a putative binding partner).

For example, methods of the present disclosure can involve evaluation of antibodies and antigen-binding fragments thereof (Ab) (e.g., comprising amino acid sequences generated by in silico design as described herein, comprising known amino acid sequences) for antigen binding, including, for example, binding specificity. Existing methods used for characterizing Ab-antigen (Ag) binding, rely heavily on techniques such as surface plasmon resonance (SPR) or enzymatic assays like ELISA, which often suffer from low throughput, linear scalability, and high requirements for polypeptide and/or putative binding partner purity and yield. Beyond those requirements, typical workflows involve the use of mammalian cells to express the polypeptide of interest for purification and usage in downstream assays, which has an associated time-cost of weeks to months prior to developing, optimizing, and running assays.

The technical solutions in this disclosure remove at least these key limitations and enables simultaneous screening and kinetic characterization of polypeptides and putative binding partners in relatively shorter timescales than traditional experimental methods (e.g., in 1- to 3-days). Technologies of the present disclosure can combine cell-free protein synthesis (CFPS), high-throughput microfluidics, and fluorescence correlation spectroscopy (FCS) (or any permutation thereof) to rapidly produce polypeptides (i.e., antibodies) and/or putative binding partners (e.g., antigens) within thousands of microreactors and measure polypeptide production as well as their binding kinetics to putative binding partners in real time. In some embodiments, polypeptides (e.g., antibodies) are produced by CFPS in microreactors. In some embodiments, putative binding partners (e.g., antigens) are produced by CFPS in microreactors. In some embodiments, polypeptides (e.g., antibodies) and putative binding partners (e.g., antigens) are produced by CFPS in microreactors. In embodiments wherein only one of polypeptide or putative binding partner are produced by CFPS in microreactors, the other (e.g., polypeptide or putative binding partner) may be introduced into the microreactor by contacting the microreactor with a carrier fluid comprising either the polypeptide or the putative binding partner. In some embodiments, both the polypeptide and the putative binding partner are introduced into the microreactor by contacting the microreactor with carrier fluid(s) comprising one or both of the polypeptide and putative binding partner. Regardless of whether the polypeptide and/or putative binding partner are produced by CFPS in microreactors or a microreactor is contacted with a carrier fluid comprising either or both of the polypeptide and the putative binding partner, such binding interaction assessment or “reactions” should be conducted under conditions wherein polypeptide-putative binding partner binding can occur. One of ordinary skill in the art will readily understand conditions wherein polypeptide-putative binding partner binding can occur and determination of such conditions. Such conditions can include selection of, for example, incubation time, salt concentrations, temperature, variation of cationic and anionic solutions, pH, reducing conditions, oxidative conditions, inclusion and/or exclusion of, for example, additional peptides (e.g., post-translational modifying enzymes), non-specific covalently modifying small molecules, and/or lipids (e.g., nanodiscs, bicelles, telodiscs).

Without wishing to be bound by any one theory, it is understood that the flexibility of this system stems from, at least in part, the use of CFPS, where any compatible polynucleotide can be converted into polypeptide and/or putative binding partner within hours, bypassing the need for mammalian cell-based expression. Non-linear scaling is achieved through microfluidic encapsulation of CFPS components and a polynucleotide(s) encoding one or more polypeptides and/or one or more putative binding partners to generate independent discrete partitions (e.g., microreactors) containing polypeptide and/or putative binding partner-producing reactions. Readouts from optical interrogation methods, such as FCS or Fluorescence Resonance Energy Transfer (FRET), enable rapid evaluation of any nascent polypeptide-putative binding partner complexes forming within the microreactors, enabling reading of binding interactions in real-time. In some embodiments, wherein technologies of the present disclosure utilize robust fluorescent signal detection for interrogating pico- to nanoliter-scale microreactor volumes, reagent usage can be minimized while still maximizing signal to noise.

A bottleneck for rapidly designing certain BCMs, such as antibodies (e.g., therapeutic antibodies), is the speed with which experimental evaluations can be performed. This disclosure is directed to an engineered platform for performing polypeptide and/or putative binding partner production and binding/affinity measurements in a fraction of the time required by traditional experimental methods, without a requirement for large experimental datasets for enabling accurate predictions. Thus, in some embodiments, this platform can produce up to 10³-10⁴ binding/affinity measurements in under three days allowing a computational-experimental approach to produce quality polypeptide (e.g., BCM) designs and experimental feedback in weeks rather than several months. This platform can be broadly applicable beyond, for example antibody development, including functional polypeptide analysis and detection. This can be accomplished by integrating cell-free protein synthesis, which provides rapid, reliable, on-demand production of polypeptide and/or putative binding partner, with engineered automated encapsulating systems that can be used to interrogate polypeptide function. This technical solution can computationally design polypeptides, such as antibodies (e.g., recombinant single chain antibody fragments (scFv)) utilizing in silico design for production and affinity testing to putative binding partners using high-throughput cell-free protein synthesis techniques for producing putative binding partners, polypeptides and/or and larger complexes known to exist and form interactions between polypeptides and putative binding partners. Furthermore, cell-free protein synthesis can be engineered as part of an additive manufacturing for high-throughput production and selection of polypeptides (e.g., antibodies) of interest based on binding affinities to putative binding partners. This technical solution includes, for example, at least computational selection and modeling of scFv and epitope interactions, engineered generation of predicted recombinant scFvs, and kinetic characterization of complex interactions using labeled affinity reagents coupled to biophysical methods such as fluorescence correlation spectroscopy within an integrated device.

This technical solution is directed at least to an in-silico polypeptide (e.g., antibody) design platform that is based on leveraging the strengths of experiment-driven, data-driven, and theory-driven approaches to mitigate the limitations of each. The initial machine-learning predictions from this platform are based on integrating existing experimental data, structural biology and bioinformatic modeling, and molecular simulations on high performance computing. For example, the technologies of the present disclosure iteratively improve its predictions by experimentally evaluating polypeptide (e.g., antibody) designs binding to putative binding partners and incorporating the binding results into the machine learning model in a feedback loop. Thus, this technical solution can enable the feedback loop from experimental evaluations to occur in about 3 days, rather than weeks or months required by conventional experimental approaches, thereby enabling high quality polypeptide (e.g., antibody, e.g., antibodies for use as BCMs) designs in weeks rather than months.

This technical solution can achieve improvements in sequence design from DNA to protein, including functional validation, in potentially under 3 days. Instead of random libraries of polypeptides, such as antibodies or antibody fragments, amino acid sequences computationally designed to have high affinity toward putative binding partners of interest, can be used to generate a library of polypeptides (e.g., antibodies or antibody fragments, such as scFv) and corresponding putative binding partners. For example, this technical solution can pursue antibodies directed against the surface antigens of viruses, including viruses of national and global concern (e.g., SARS-COV-2, Ebola, and Venezuelan Equine Encephalitis Virus (VEEV)). In some embodiments, existing techniques for recombinant antibody production can be combined with cell-free protein synthesis techniques, potentially for producing not only polypeptides, antibodies, or fragments thereof (e.g., single-chain antibodies), but also the computationally-predicted epitopes, full-length protein antigens, or even larger protein complexes in a single microreactor. For example, this technical solution can express large numbers (103-105) of polypeptide (e.g., antibody)/putative binding partner (e.g., antigen) pairs at once. This can be accomplished using engineered micro-encapsulation techniques that can also allow one to compartmentalize production and functional assessment using spectral fluorescent techniques that require small volumes or low concentrations of solutes. This can also dramatically reduce the cost and possibly the time to generate polypeptides that bind to putative binding partners (e.g., antibodies, e.g., antibodies for use as a BCM).

This technical solution can establish an iterative loop for predicting, generating, evaluating and refining polypeptide design (e.g., amino acid sequences) in under three days using a microfluidic system. This technical solution can also include or integrate with a biological additive manufacturing solution for rapid development of key BCMs needed in biodefense and/or health.

An aspect of this disclosure is directed to generating polypeptide design candidates (e.g., predicted amino acid sequences of a polypeptide) using technologies of the present disclosure based on machine learning and simulation. This technical solution can use an in-silico machine-learning-driven polypeptide design platform to generate initial polypeptide designs for particular putative binding partners of interest (e.g., antigens). Further, throughout the proposed effort, this technical solution can incorporate binding and affinity measurements from the proposed system iteratively so that each subsequent iteration provides improved polypeptide (e.g., antibody) designs for testing performance. This platform is applicable to the design of, for example, full-length antibodies (e.g., against viral antigens such as SARS-COV-2 S-protein), performing over one hundred thousand in-silico binding computations in a few days. This can be adapted for predicting and analyzing for example, scFvs, for example, against SARS-COV-2, as well as other viruses of interest, and keeping pace with the potential speed at with the system this technical solution are proposing.

An aspect of this disclosure is directed to microencapsulation development and integration for production of polypeptides and/or putative binding partners. To generate polypeptides (e.g., antibodies, scFvs), this technical solution can use cell-free protein synthesis, to bypass polypeptide production procedures such as tissue culture-based hybridoma or animal immunization techniques. Cell-free protein synthesis can utilize a plurality of types of cell lysates, including, for example, E. coli lysates. To enable high-throughput screening, this technical solution can also develop biologically compatible micro-encapsulation techniques on a microfluidic card comprising the cell-free lysates and a polynucleotide(s) encoding one or more polypeptides (e.g., with amino acid sequences generated from in silico iterative design, e.g., as described herein) and/or one or more putative binding partners. In some embodiments, technologies of the present disclosure start with polypeptides with known amino acid sequences that bind to specific putative binding partners as a test case. By leveraging the monodisperse microreactor production capability of a microfluidic system, microencapsulated cell-free protein synthesis can be carried out in large numbers (>10⁵ unique polypeptides or putative binding partners). During production, immiscible fluids can be utilized in a microfluidic device to create emulsions (e.g., microreactors). This solution can create lipid compartments or use “Ribo-beads” (commercially available microbeads coated with modified ribosomes, see, U.S. patent application Ser. No. 16/752,222, incorporated herein by reference) within a microreactor. Ribo-beads may be used to limit protein translation activity to a specific area within a microreactor. This may enhance the optical detection sensitivity of polypeptide synthesis and interactions with putative binding partners by providing distinct space for separating transcription and translation from spatial area needed for detection. The core fluid can be surrounded by a shell layer that is co-generated and solidified through UV-activated polymerization to form a microcapsule to provide a temporary compartment for facilitating measurements of specific polypeptide-putative binding partner (e.g., antibody-antigen) pairs.

An aspect of this disclosure is directed kinetic characterization of polypeptide-putative binding partner interactions using fluorescence correlation spectroscopy and/or validation of virus neutralization for top polypeptide (e.g., BCM) candidates. Rapid characterization of polypeptide-putative binding partner interactions can use Fluorescent Correlation Spectroscopy (FCS) to characterize interactions. FCS is capable of measuring single molecular diffusion statistics directly in solution and providing in situ measurements for both qualitative and quantitative assessment of complex interactions in solution. This technique is capable of assessing single molecule level interactions at a temporal and spatial level that is ideal for characterizing affinity reagents, protein complexes and individual protein structure dynamics. Also, this technical solution instrumentation is capable inferring topology and monodispersity for affinity bound complexes of polypeptides and putative binding partners.

FCS is based on the analysis of time correlations in fluorescence fluctuation emitted when fluorescently labeled molecules diffuse in and out of an observation volume. The fluorescent emitted from fluorescently labeled molecules (e.g., fluorescently labeled polypeptide and/or putative binding partner) in an observation volume (e.g., in an interrogation chamber) is measured by microscopy methods (e.g., confocal microscopy). Fluorescence intensity is related to the concentration of folded, fluorescently labeled molecule (e.g., polypeptide, putative binding partner) and binding between polypeptide and putative binding partner is related to how much diffusion time shifts compared to each molecule's diffusion time individually (e.g., fluorescently labeled polypeptide diffusion time without binding to the putative binding partner). An increase in binding affinity (e.g., a lower K_{D}) provides a relatively larger shift in the diffusion time upon binding. For example, a smaller fluorescently labeled antigen has a relatively faster diffusion time compared to an antigen specifically bound by antibody. FCS is a highly quantitative method based, at least in part, on how well a polypeptide interacts with a given putative binding partner on average.

Dual-color FCS, wherein, for example, a polypeptide and a putative binding partner are labeled with spectrally distinct fluorophores, can be used to provide diffusion data for both the polypeptide and the putative binding partner. Upon excitation of the spectrally distinct fluorophores, the fluorescence from the two-color fluorophores is separated and counted into two detection channels, respectively. Compared with the FCS using a single fluorescent label (“single species”), dual-color FCS is neither limited by the relative size change of the complex (e.g., polypeptide-putative binding partner complex) compared with single species nor susceptible to binding with other species. See, e.g., Yu, L. et al., “A Comprehensive Review of Fluorescence Correlation Spectroscopy”, Front. Phys., 12 Apr. 2021, Vol. 9, incorporated herein by reference.

Three-color FCS can be utilized to decrease the signal-to-noise ratio of measurements. Without wishing to be bound by any one theory, it is understand that three-color FCS decreases the signal-to-noise ratio because a narrower wavelength is available for each channel and cross talk between the channels will be larger. Three-color FCS can be useful in accordance with technologies of the present disclosure wherein the putative binding partner comprises a combination of molecules, such as a complex of polypeptides. See, e.g., Hwang LC et al. “Simultaneous multicolor fluorescence cross-correlation spectroscopy to detect higher order molecular interactions using single wavelength laser excitation.” Biophys J. 2006 Jul. 15;91(2):715-27, incorporated herein by reference.

In some embodiments, the one or more polypeptides for use in accordance with technologies of the present disclosure are fluorescently labeled. In some embodiments, the one or more putative binding partners for use in accordance with technologies of the present disclosure are fluorescently labeled. In some such embodiments, wherein either the polypeptide or the putative binding partner in a given microreactor is fluorescently labeled, single species FCS can be utilized.

In some embodiments, the one or more polypeptides and the one or more putative binding partners for use in accordance with technologies of the present disclosure are each fluorescently labeled with a spectrally distinct fluorophore. In some such embodiments, wherein both the polypeptide and the putative binding partner in a given microreactor is fluorescently labeled, dual-color FCS can be utilized.

In some embodiments, wherein the one or more putative binding partners comprises a complex of polypeptides, each of the one or more polypeptides and each polypeptide of the putative binding partner complex for use in accordance with technologies of the present disclosure are fluorescently labeled with a spectrally distinct fluorophore. In some such embodiments, three-color FCS can be utilized.

Technologies of the present disclosure can be integrated for use with a fluidic card to automate the imaging (e.g., FCS) technology, facilitating high-throughput measurements within individual microreactors. Upon identification of top polypeptide (e.g., BCM) candidates that have been validated as having high affinity for their directed putative binding partner (e.g., antigen), this technical solution can evaluate them for the ability to, for example, neutralize their viral target.

This disclosure is directed to, among other things, a robust pipeline for producing and characterizing polypeptides (e.g., antibodies) in an encapsulated format for assessing biochemical interactions of sensitivity and specificity for binding to putative binding partners (e.g., antigens). This technical solution can comprise evaluating an initial set of polypeptides (e.g., comprising amino acid sequences generated by in silico design, polypeptides with known amino acid sequences) for their binding affinity to one or more putative binding partners. In silico refinement of 10²-10⁴ polypeptide designs through multiple rounds of improved predictions can be produced by incorporating one or more binding affinities of the polypeptides to one or more putative binding partners into a machine learning model (e.g., a neural network, a random forest, a support vector machine, a transformer model, a large language model, etc.). This technical solution can develop a microfluidic card for encapsulation of cell-free protein synthesis and use a FCS approach for rapidly characterizing the sensitivity and specificity of an initial set of binding affinities to be incorporated into the machine learning model. These polypeptides are expected to have nM or better affinities and be produced and subsequently selected for affinity enhancements in under three days. This solution can deliver a high-throughput version of the protocol for efficient production and screening for at least 10³-10⁴ interactions in a microfluidic platform per day. FCS can require the use of fluorescent labels on different components (e.g., polypeptides, putative binding partners) used in the technologies of the present disclosure and as described elsewhere herein. Thus, this solution can employ fluorescent labels (e.g., green fluorescent protein (GFP) or variants thereof) fused to polypeptides and/or putative binding partners.

The current in-silico machine-learning-driven polypeptide (e.g., antibody) design platform can be adapted for use single chain antibodies that focuses on the antigen recognition sequence. As the proposed engineering platform begins to produce higher volumes of data in shorter time scales, the most expensive components of the in-silico platform may begin to become bottlenecks. This can require adapting this computation process to rely on faster running molecular dynamics simulations or rely more on the machine learning model. This solution can also leverage extensive literature and open source code that is available that implements approximate Gaussian Process Model approaches capable of scaling to significantly larger datasets with high accuracy.

This technology can have multiple applications including research, polypeptide (e.g., antibody) design (e.g., for use as a BCM), and in pathogen detection. To rapidly accelerate BCM design, these aforementioned techniques for polypeptide (e.g., antibody) and/or putative binding partner production can be combined with cell-free protein synthesis techniques, potentially for producing putative binding partners (e.g., antigens) and other proteins that make up larger complexes known to exist and form, for example, interactions between host or pathogen membranes. Using microencapsulation, cell-free protein synthesis can be highly compartmentalized for rapid production of polypeptides (e.g., antibodies or antibody fragments) in a high-throughput format that may be amenable to automated analysis. This method can start from DNA to synthetically produce certain required components for evaluating polypeptide-putative binding partner (e.g., antibody-antigen) interactions in a single microreactor. This may be ideal for cell-free polypeptide and/or putative binding partner production, which can be scaled down and conducted at nanoliter reaction volumes, thus dramatically reducing the cost and possibly the time to generate these polypeptides (e.g., antibodies) and/or putative binding partners (e.g., antigens). Ultimately, this strategy can remove a substantial obstacle for the generation and testing of polypeptides (e.g., antibodies, scFvs) and can theoretically reduce the production time down to hours instead of the several month time it takes to normally generate polypeptides that specifically bind a putative binding partner, including, for example, antibodies that specifically bind an antigen.

In some embodiments, polypeptides (e.g., antibodies) are produced by CFPS in microreactors. In some embodiments, putative binding partners (e.g., antigens) are produced by CFPS in microreactors. In some embodiments, polypeptides (e.g., antibodies) and putative binding partners (e.g., antigens) are produced by CFPS in microreactors. In embodiments wherein only one of polypeptide or putative binding partner are produced by CFPS in microreactors, the other (e.g., polypeptide or putative binding partner) may be introduced into the microreactor by contacting the microreactor with a carrier fluid comprising either the polypeptide or the putative binding partner. In some embodiments, both the polypeptide and the putative binding partner are introduced into the microreactor by contacting the microreactor with carrier fluid(s) comprising one or both of the polypeptide and putative binding partner. Regardless of whether the polypeptide and/or putative binding partner are produced by CFPS in microreactors or a microreactor is contacted with a carrier fluid comprising either or both of the polypeptide and the putative binding partner, such binding interaction assessment or “reactions” should be conducted under conditions wherein polypeptide-putative binding partner binding can occur. One of ordinary skill in the art will readily understand conditions wherein polypeptide-putative binding partner binding can occur and determination of such conditions. Such conditions can include selection of, for example, incubation time, salt concentrations, temperature, variation of cationic and anionic solutions, pH, reducing conditions, oxidative conditions, inclusion and/or exclusion of, for example, additional peptides (e.g., post-translational modifying enzymes), non-specific covalently modifying small molecules, and/or lipids (e.g., nanodiscs, bicelles, telodiscs).

Direct analysis of polypeptides by biophysical methods within cell-free protein synthesis reactions can further accelerate the BCM design pipeline. For example, a three color-based Fluorescent Correlation Spectroscopy (FCS) instrument to characterize biological molecules can be utilized. This technical solution can use similar methods in this disclosure to examine binding interactions between polypeptides and putative binding partners directly within cell-free protein synthesis reactions (e.g., in a microreactor). Coupling high-throughput production with efficacy testing can create a rapid experimental setup that can be useful for the validation of, for example, in silico predicted BCMs, thus providing a technical improvement in responding to emerging global biothreats.

Instead of random libraries of, for example, antibodies, amino acid sequences computationally designed by in silico methods of the present disclosure to have high affinity toward putative binding partners (e.g., antigens) of interest, can be used to generate a libraries of polypeptides and corresponding putative binding partners. This technical solution can pursue, for example, antibodies directed at least against the surface antigens of viruses. Such viruses may include, for example, viruses of national and global concern, such as SARSCOV-2, Ebola, and Venezuelan Equine Encephalitis Virus (VEEV). In some embodiments, recombinant antibody production can be combined with cell-free protein synthesis techniques to produce not only antibodies or antibody fragments (e.g., single chain antibodies), but also the computationally-predicted epitopes, full length protein antigens, or even larger protein complexes in a single microreactor. In some embodiments, large numbers (10³-10⁵) of antigen/antibody pairs are expressed at once using engineered micro-encapsulation techniques that can also allow for compartmentalized production. This compartmentalization can achieve functional assessment on discrete pairs of antibodies and antigens using spectral fluorescent techniques (e.g., FCS) that require small volumes or low concentrations of solutes, dramatically reducing the cost and possibly the time to generate polypeptides (e.g., antibodies) with improved binding to putative binding partners (e.g., antigens).

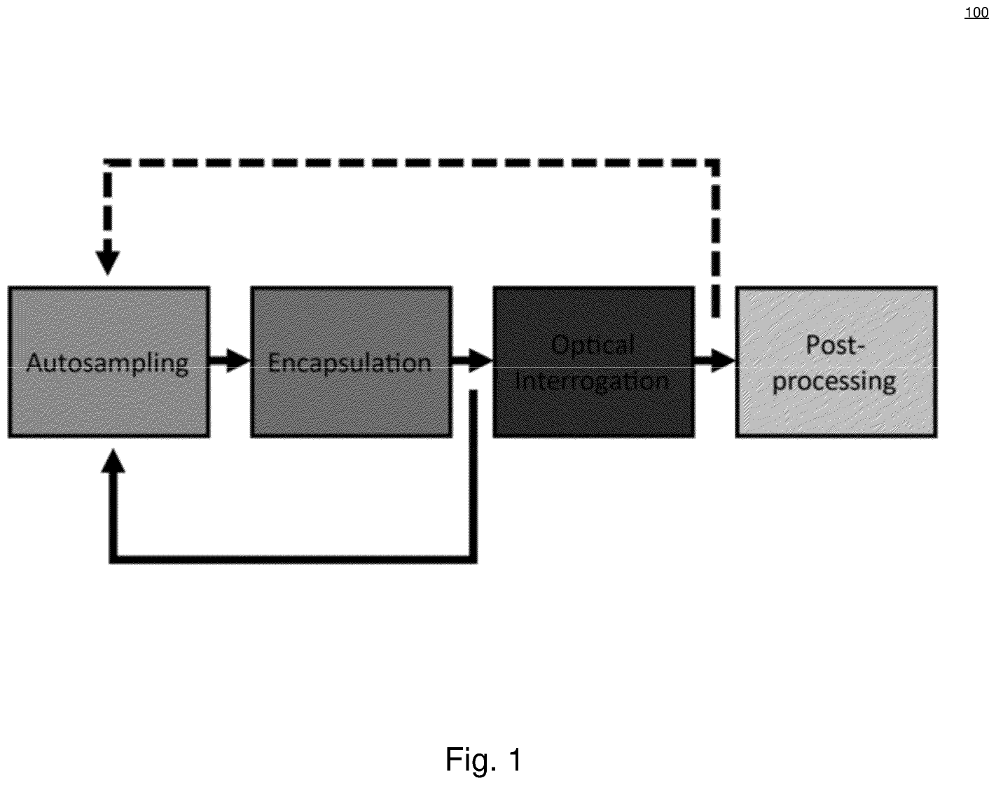

FIG. 1 depicts an example system (“platform”), according to this disclosure. The high throughput screening process can be generally described, by way of example, by the flow chart in FIG. 1. A fluidic robot or autosampler in the autosampling step creates an array of samples comprising CFPS components to produce one or more polypeptides and/or one or more putative binding partners and fills them in the encapsulation step, which partitions each sample into a plurality of microdroplets or microcapsules (referred to collectively as “microreactors”). In some embodiments, wherein only one of polypeptide or putative binding partner are produced by CFPS in microreactors, the other (e.g., polypeptide or putative binding partner) may be introduced into the microreactor by contacting the microreactor with a carrier fluid comprising either the polypeptide or the putative binding partner. In some such embodiments, contacting can occur within an interrogation chamber. Regardless of whether the polypeptide and/or putative binding partner are produced by CFPS in microreactors or a microreactor is contacted with a carrier fluid comprising either or both of the polypeptide and the putative binding partner, such binding interaction assessment or “reactions” should be conducted under conditions wherein polypeptide-putative binding partner binding can occur (e.g., as described herein). These microreactors are then flowed into and held inside an interrogation chamber prior to the optical interrogation step, where their positions are tracked while they are periodically interrogated for assay activity (e.g., binding activity to putative binding partners, e.g., by FCS). This enables real-time, high-precision monitoring of thousands to tens of thousands of individual reactions. Once the interrogation segment concludes on a batch of microreactors, the microreactors of interest (e.g., microreactors comprising the best performers, e.g., microreactors comprising polypeptides with relatively high or highest binding affinity to their putative binding partner, e.g., microreactors comprising polypeptides that bind to a putative binding partner) may be selectively sorted from the general population during the postprocessing step for further interrogation or correlative analysis with other measurement modalities (e.g., assessment of neutralization activity of a given polypeptide).

The loopback between the autosampling and encapsulation steps illustrates that these steps may be repeated as needed to fill the interrogation chamber with a plurality of microreactors containing different polypeptides and/or putative binding partners before the interrogation segment begins. This multiplexing is made possible through timestamping a particular microreactor's encapsulation and position within the interrogation chamber. Alternatively, the interrogation chamber may be continuously loaded with newly encapsulated microreactors and purged of analyzed ones (e.g., with low binding affinity to their putative binding partner) to create a continuous interrogation model (e.g. as illustrated by the dashed loopback).

Examples of autosampling can include, but are not limited to, the following. The autosampling step may use a fluidic robot or autosampler (both henceforth may be referred as “fluid handlers”) to transfer, mix, or dilute previously prepared samples (i.e., on a mother plate) with other samples or reagents to create daughter plates of unique reactions that can be partitioned into microreactors in the Encapsulation step. Samples and reagents may be liquid or solid, that latter of which can be resuspended in a reagent prior to partitioning. Samples may be prepared in containers, such as tubes or multi-well format plates (e.g., 96-well plates, 384-well plates). The fluid handler may heat or cool samples based on the particular need of the assay (e.g., to provide conditions wherein polypeptide-putative binding partner binding can occur).

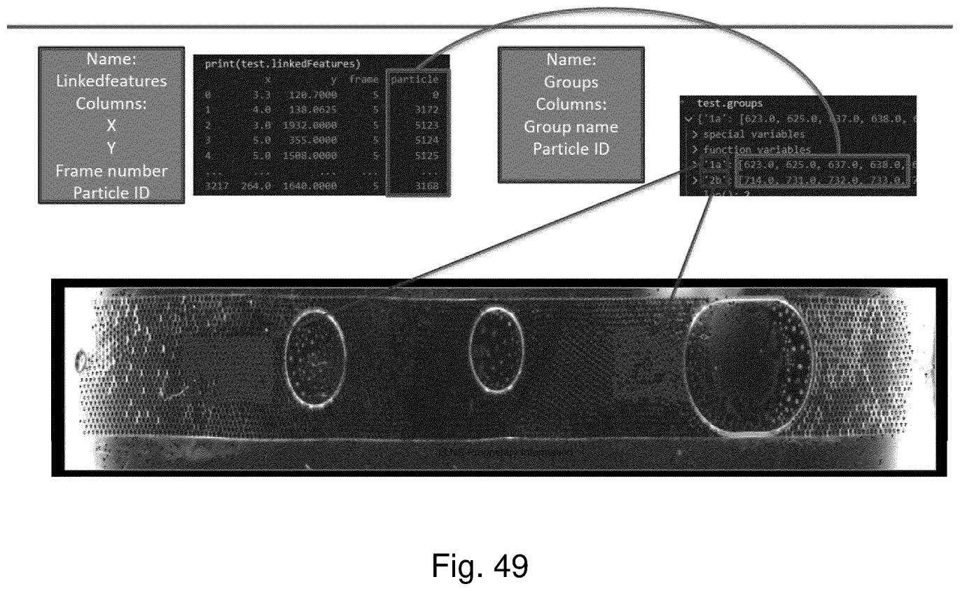

In some embodiments, a user or a machine learning model (e.g., a neural network, a random forest, a support vector machine, a transformer model, a large language model, etc.) may select multiple regions of interest (ROIs) within the interrogation chamber, as depicted in FIG. 49. The droplets within these ROIs would be selectively tracked over time. For example, a user may provide an input highlighting, indicating, or identifying different areas of the channel of the interrogation chamber (e.g., when the droplets are in the channel of the interrogation chamber). The system may receive the input and identify the microdroplets that are in the input regions of interest. The system can assign identifiers (e.g., droplet identifiers or particle identifiers) to each of the microdroplets in the regions of interest that individually identify each of the droplets. The system can identify the positions (e.g., XYZ positions) of the microdroplets or particles within the channel of the interrogation chamber and store the identified positions in a data structure over time. Examples of the data structure in which the microdroplets or particles may be stored are illustrated in FIG. 49. In this way, the system can track the movement of individual droplets within the channel. Additionally, by selecting subsets of droplets to monitor, the system can reduce the processing resources that are required to process the data of the droplets by avoiding processing the data of every droplet in the channel.

In another example, the system can execute or use a machine learning algorithm (or machine learning model) to select regions of interest. For instance, the system can execute or use a machine learning algorithm that has been trained to select microdroplets from the microdroplets within the channel of the interrogation chamber. The machine learning algorithm may be trained to select microdroplets that will most accurately indicate binding affinities to putative binding partners. The system can train such a machine learning algorithm using a supervised or an unsupervised training technique. For instance, the system can obtain or receive training data including images of microdroplets within the channel of the interrogation chamber and/or one or more channels of other interrogation chambers. The images can be labeled to indicate the correct or ground truth microdroplets to select regions of interest to select for monitoring. The system can feed the labeled images into the machine learning algorithm and train the machine learning algorithm using backpropagation techniques based on differences between microdroplets that the machine learning algorithm selects and the labeled microdroplets of the images. The system can train the machine learning algorithm in this way over time with one or more training datasets. The system can train the machine learning algorithm until determining to deploy the machine learning algorithm, such as based on the machine learning algorithm having an accuracy above a threshold. By training the machine learning algorithm in this way, the system can improve the selection of the microdroplets to use to determine binding affinities to putative binding partners while still avoiding processing of each droplet in the channel.

In some cases, the system can determine or select regions of interest based on the constituents of microdroplets within the channel. For example, the system can receive as input the constituents of microdroplets within the chamber. Examples of such constituents can include the types of polypeptides of the microdroplets and/or putative binding partners in the chamber. The system can include identifications of the types of polypeptides and/or putative binding partners in a feature vector with one or more images of the chamber. The machine learning algorithm may be trained to select regions of interest based on such identifications and images of channels. The system can execute the machine learning algorithm based on the feature vector to select a region of interest and/or microdroplets within the channel. In doing so, the machine learning algorithm may more accurately and/or more quickly identify microdroplets to analyze to determine or predict amino acid sequences.

For example, the system can receive as input (e.g., from a client device) one or more identifications of a particular set of known viral proteins and a particular set of plasmids that would encode candidates for an Ab to that set of viral proteins. The system can input the identifications with one or more images of the channel into the machine learning algorithm. The system can execute the machine learning algorithm to generate or identify a region of interest based on the images and identifications. Because the machine learning model may determine the region of interest based on the identifications, the machine learning model may more accurately and/or more quickly identify regions of interest that include microdroplets to use to determine binding affinities to putative binding partners. For example, the output could show brighter fluorescence if using a FRET assay or better binding affinity if doing an FCS assay.

The system can use another machine learning algorithm (e.g., a second machine learning algorithm) to determine one or more predicted amino acid sequences of a polypeptide. For example, the system can determine one or more binding affinities of the microdroplets within the chamber by monitoring the microdroplets identified from the region of interest within the chamber. The system can generate a feature vector including the determined (or measured) binding affinities with the identifications of the types of viral proteins and plasmids. The system can execute the second machine learning algorithm using the feature vector as input to output predicted amino acid sequences of a polypeptide. By using the binding affinities in combination with the types of the microdroplets, the system can avoid follow-up sequences to ascertain what was in the droplets, making the process substantially faster. Such may be useful, for example, when performing a 1:1 assay and/or when performing a pooled screening approach vs. a totally stochastic needle-in-a-hay-stack approach in which a downstream analysis is needed to determine what amino acid sequences gave a positive result.

Examples of encapsulation can include, but are not limited to, the following. After making daughter plates, the fluid handler withdraws a known sample volume from the desired well and injects it into the input of the encapsulation system. This may be performed by a sample injection valve that accepts the needle or pipette tip of the fluid handler, when, operated in one state, can create a fluidic connection that allows the handler to infuse the known sample volume into the valve's sample holding coil. The stator of the valve may then be switched to allow continuously flowing carrier fluid (originally bypassing the sample coil) to push the newly loaded sample into the stream that can turn into the inner core of the microreactors. Although a two-position injection valve is one example method of injecting samples, other can be additionally or alternatively used. For example, one-way valves may be used, to enable the fluid handler to inject the sample directly into the inner core stream without a sample coil and injecting directly into a novel open fluidics architecture that takes advantage of surface tension forces to allow fluids to flow on the outside of a structure (vs. inside tubing).

For example, microreactors can be created by isolating discrete volumes of aqueous sample in water-immiscible carrier solutions. Microdroplets with high monodispersity in size can be created at microfluidic junctions (planar or concentric) where water and oil meet. The microfluidic junctions may be created through microfabrication practices (lithography, etching, epoxy molds, etc.), through precision pulling and concentric alignment of glass capillaries, or other means. The flow rate of oil (or water-immiscible carrier fluid) is larger than that of the aqueous input to create aqueous droplets in oil carrier fluid (or W-O). Microcapsules can be created by using the generated microdroplets as an inlet to another junction that uses aqueous as the other inlet. This creates a water-in-oil-in-water (or W-O-W) double emulsion that can be compatible with systems that need an aqueous carrier fluid, but still have an aqueous core. Flow rates for each fluidic inlet can be independently modulated using various pumps (peristaltic pumps, syringe pumps, pneumatic pumps, etc.). Another benefit of multiple emulsions is the water-immiscible shell may be polymerized via thermal, ultraviolet, or chemical methods to create mechanically robust microcapsules to store sub-nanoliter samples for long periods. Additional inlets may be used to create multi-core capsules. For microdroplets or unpolymerized microcapsules, a surfactant is used to ensure stability of microreactors so that they do not re-coalesce enroute to or inside of the interrogation chamber.

To rapidly increase screening scope, the fluid handler may inject a mixture comprising one or more polypeptides (or polynucleotides encoding the one or more polypeptides) and/or a mixture comprising one or more putative binding partners (or polynucleotides encoding the one or more putative binding partners) into two ports or valves that direct the mixtures toward a microfluidic junction that mixes the two mixtures immediately before encapsulation. The number and concentration of putative binding partners in the putative binding partner mixture and that of the polypeptides in the polypeptide mixture, flow rates of each mixture, and the volume of the resulting microreactor can all be optimized to stochastically force microreactors to contain a single polypeptide (or polynucleotide encoding a single polypeptide) and a single putative binding partner (or a polynucleotide encoding a single putative binding partner). For example, combining and encapsulating a mixture of 100 antibody candidates (polypeptide mixture) with a mixture of 100 antigens (putative binding partner mixture) can result in a single screen of 10,000 antibody-antigen combinations. Microreactors containing positive antibody-antigen activity (e.g., binding, e.g., relatively high binding affinity to their putative binding partner) can be retained to identify the antibody and antigen amino acid sequences.

Microreactors may be created using other methods other than microfluidics, but this generally results in a wider distribution of sizes that may add variation in downstream analysis. Applicable methods to make batches of microreactors include, for example, shaking, sonicating/stirring, or homogenizing two immiscible fluids to create emulsions, which can be stabilized with surfactant to prevent re-coalescence. Subsequent shaking or homogenization steps with additional fluids for each step can create microreactors with increasing numbers of cores. Although most microreactors may have an aqueous core for the application space denoted in this disclosure, some applications may require a water-immiscible cores. Encapsulation methods can be modified to ensure reliable creation of these oil core microdroplets (O-W) or O-W-O microcapsules. If additional partitioning is not necessary for the application, microreactors can be realized using the fluidic handler to inject the sample into an immiscible stream thus creating segmented flow. In this case, microreactor volumes can be limited by the minimum ejection volume of the fluid handler.

Examples of optical interrogation can include, but are not limited to, the following. Upon first entering the interrogation chamber, each microreactor is detected using machine vision and entered into the control program's database. Each microreactor is given a unique identifier that links time sensitive data such as current centroid position (i.e., X, Y position of microreactor center) in the chamber, diameter, volume, circularity, and optical interrogation data (e.g., fluorescent signal strength, affinity constant, diffusion constant, etc.) to that particular microreactor. For example, a unique identifier is a text, numeric, or alphanumeric string that is assigned to each droplet, and stored as an entry, field or object in a table, class, or database structure. A table, class, or structure records this data for every captured and analyzed video frame. Tracking may be realized through a camera that is positioned above or below the chamber and has a lens that allows it to view the entire chamber. A light of white or other spectrum may be employed to illuminate the chamber to enhance the contrast of the microreactors. For accurate detection and analysis, the camera resolution and lens magnification can be chosen to ensure the microreactors are resolved by at least ˜10 pixels. Microreactors may be detected using machine vision as discussed herein, including by a controller as discussed herein. For example, the controller can include a tracking module developed with the python language. Particles' locations can be identified via a “locate” method for each frame. Once location data is retrieved, this data may be used to track particles across frames via a “correlator” class. Video capture rates are optimized based on various factors, including microreactor velocity, camera resolution, and computer processing power, with the goal of providing real-time microreactor position tracking and interrogation data. In some embodiments, optical interrogation includes the use of FCS. This technical solution is not limited to optical interrogation, and detection of movement by means other than optical interrogation is contemplated. For example, the interrogation can be performed via electromagnetic signals in spectra distinct from visual spectra. For example, the interrogation can be performed via non-electromagnetic response in spectra distinct from electromagnetic signals in visual spectra.

Microreactors are loaded into the chamber until they reach a gate that prevents them from exiting the chamber (while allowing fluid to exit). The gate is designed to be integrated into the interrogation chamber and is actuated to rapidly open or close to ensure control of microreactor release and retention with high temporal resolution. This gate may be realized by using positive or negative dielectrophoretic (DEP) or electrophoretic forces, optical forces, pneumatic or fluidic pressure to collapse a flexible ceiling magnetic forces, by electromechanical means such as a physical gate that spans the width of the chamber, etc. The chamber continues to fill with microreactors until it is completely full, or until a user or program stops filling the chamber. A permanent gate (e.g., mechanical filter) or permanently actuated gate may be used in place of a dynamic gate to keep the microreactors from exiting the chamber. In this embodiment, flow in the chamber can be reversed to wash the microreactors back out the chamber. Once chamber loading is complete, flow into the chamber can be stopped or slowed to ensure microreactors do not drift back out the chamber entrance.

Optical interrogation of the microreactors then commences within the chamber. An objective is used to focus an excitation signal within the aqueous core of each microreactor and digitize the resulting emission signal (via sensitive CMOS or CCD digital camera), which is recorded for its unique microreactor identifier. Interrogation occurs serially until all microreactors within the chamber are scanned, resulting in one interrogation datapoint for each microreactor. Scanning may be repeated until the assay is complete, providing real-time data over the span of hours, days, or even weeks. To accomplish optical scanning, the objective may be mounted on a motorized XY or XYZ stage that can allow it to translocate to each microreactor's recorded centroid. Alternatively, the objective may be fixed with the interrogation chamber being translocated by an XY or XYZ stage to center each microreactor within the focus of the objective. A combination of staging may be used to translocate both objective and chamber, for example, a Z stage to automatically focus the objective and an XY stage to move the chamber.

The interrogation chamber may be composed of a combination of glass, metal, plastic, rubber, polymer, or other materials that are compatible with the carrier solution of the microreactors. One face of the chamber may be transparent, where both the machine vision camera and the optical interrogation objective are on the same side of the chamber. In this case, a combination of optical filters, mirrors, lenses, and side illumination may be employed to ensure the machine vision optics and light source do not interfere with the interrogation objective. A chamber that has a transparent ceiling and floor may be used to create a simpler optical setup. In this case, the machine vision camera can track particles from above the chamber, while the objective can interrogate the particles from below the chamber or vice versa. The light source may be placed above or below the chamber to provide reflected or transmitted illumination of the microreactors, respectively.

The geometry of the fluidic channel(s) that compose the interrogation chamber may take various forms based on the application. The chamber may take the form of a large, monolithic channel with a width that expands to reduce the average flow velocity to allow optical analysis of large populations of microreactors. The chamber can be a long, serpentine channel that forces the microreactors into single file line, or may comprise a network of fluidic traps that can allow global or selective release of microreactors. Heating or cooling the chamber may be needed to accelerate reactions or preserve the microreactors during longitudinal studies, and can be employed via various methods, such as resistive heating, infrared heating, Peltier cooling, and controlling the temperature (and humidity) of the ambient around the chamber. Once optical interrogation is complete, the gate is deactivated, and flow is restarted/increased to wash out the interrogated microreactors. Afterwards, the gate is re-activated and new microreactors are loaded into the chamber for another round of interrogation. In an embodiment, flow through the chamber may not be stopped, but slowed enough to allow optical interrogation to occur. In this case, a gate may not be necessary, allowing microreactors to continually pass through the interrogation chamber.

Examples of postprocessing can include, but are not limited to, the following. After passing through the gate, microreactors of interest (e.g., microreactors comprising a polypeptide with a relatively high affinity to the putative binding partner) may be selectively pulled out of the general population using various active sorting techniques. Methods may include using optical tweezers, DEP, or electrophoresis, laser-induced bubble cavitation, valves, magnetism, hydrodynamic pressure, or acoustic waves to force specific microreactors out of one flow path and into another one for downstream in situ and/or ex situ analysis.

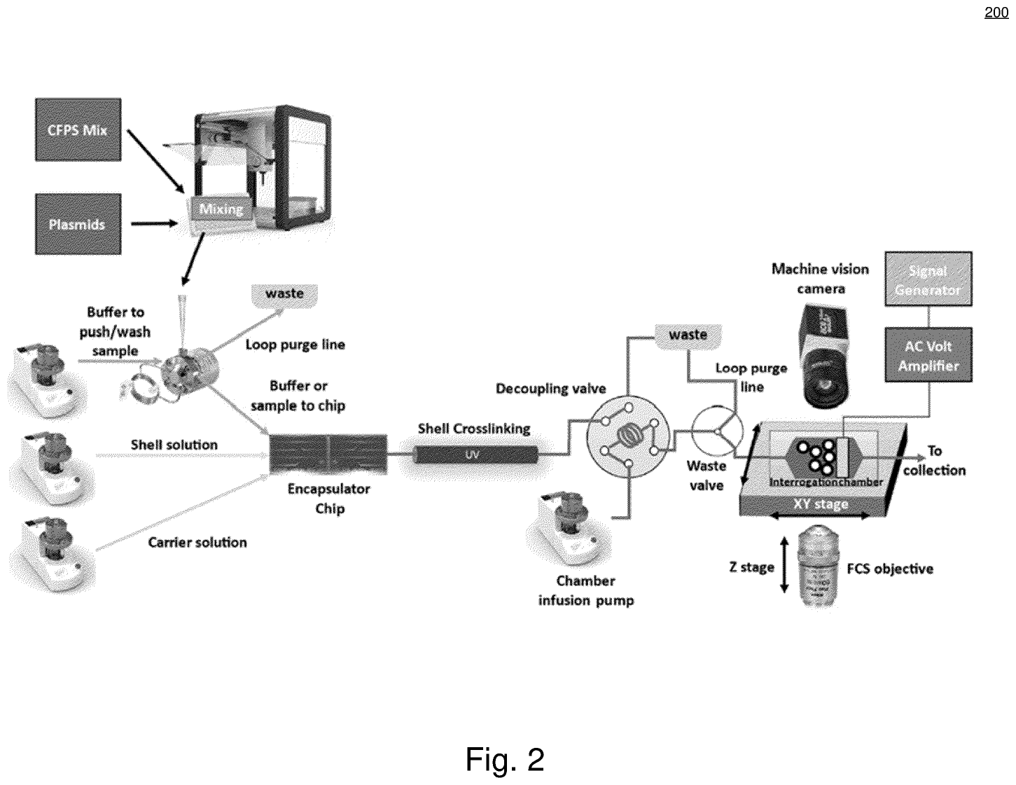

FIG. 2 depicts an example apparatus, according to this disclosure. For example, the apparatus can develop and optimize cell-free protein synthesis systems for generating polypeptides, such as native-like and recombinant forms of full-length antibodies, including in silico design of polypeptide (e.g., antibody) amino acid sequences. For example, this technical solution can use an in silico machine-learning-driven antibody design platform to generate initial polypeptide (e.g., antibodies or antibody fragments) designs for a putative binding partner. Further, this technical solution can incorporate binding and affinity measurements from the proposed system iteratively so that each subsequent iteration provides improved polypeptide (e.g., antibody or fragment thereof) designs for testing performance. The machine-learning predictions from this platform are based on integrating one or more of experimental data, structural biology, bioinformatic modeling, and molecular simulations on high performance computing. This solution can apply the machine learning platform to the design of, for example, full-length antibodies, such as full-length antibodies against SARS-COV-2 S-protein, to achieve a technical improvement of performing over one hundred thousand in silico binding computations in a few days. This technical solution can also be adapted for predicting and analyzing, for example, scFvs (e.g., scFvs against SARS-COV-2), as well as other viruses of interest and fluorescent controls of interest. The output can be a library of predicted amino acid sequences, generated by the machine learning system discussed herein, to be subsequently converted to optimized polynucleotide sequences, encoding polypeptides comprising such predicted amino acid sequences (e.g., full length antibodies or scFvs) that are predicted to recognize a specific putative binding partner (e.g., antigen), for example, with improved binding affinity. However, in some embodiments, the library can contain a series of mutations that can modulate the binding affinity of the polypeptide to a specific putative binding partner. This mutational variety can be used in combination with the high throughput analysis discussed herein, to generate a new dataset of polypeptide affinities to further train the computational model.

Aspects of the present disclosure are directed to polynucleotide(s) encoding a polypeptide(s) (e.g., predicted to specifically bind one or more putative binding partners) and/or a putative binding partner(s). Such polynucleotides can comprise a nucleotide sequence encoding a label, such as a fluorescent label (e.g., GFP, RFP, mCherry). The library of polynucleotides can be rapidly synthesized using gene synthesis instrumentation, e.g., as double stranded gene fragments. These gene fragments can be directly added to cell-free protein synthesis reactions described herein. For example, a DNA synthesis method can be used to generate multiple DNA constructs. Here, for example, pooled libraries of ˜200 base pair oligos are captured in sets of uniquely barcoded microbeads in picoliter emulsion droplets (“microreactors”). PCR and gene assembly are conducted in the microreactors to produce full length, double-stranded DNA constructs of interest. This method can produce tens of thousands of genes up to 700 bp at a low cost of <$0.70/gene. Depending on the putative binding partners (e.g., antigens) chosen, this technical solution anticipates generating initial polypeptides (e.g., antibodies, e.g., BCM candidates) in a few weeks-months. Subsequently, this solution can support ongoing iterations continuously, providing additional polypeptide candidates from the in silico machine-learning-driven polypeptide (e.g., antibody) design platform described herein that are predicted to bind to putative binding partners with improved properties, such as higher binding affinities (e.g., as compared to the originally evaluated polypeptides).

Aspects of the present disclosure are directed to cell-free lysate development for protein production (e.g., using cell-free protein synthesis). To generate polypeptides, such as antigens, antibodies and fragments thereof, this technical solution can use cell-free protein synthesis, to bypass, for example, antibody production procedures such as tissue culture-based hybridoma or animal immunization techniques. Cell-free protein synthesis reactions can utilize E. coli lysates, however, other lysate types that may enhance polypeptide yields and allow for post-translational modifications of mammalian polypeptides, including lysates originating from insect and human cell lines.

Typically, cell-free protein synthesis of proteins of interest occurs in bulk solution. For engineering this platform, this technical solution can use several techniques to spatially partition the cell-free protein synthesis reaction in order to enhance protein production and analysis. For example, commercially-available microbeads can be coated with modified ribosomes to produce “Ribo-beads.” These “Ribo-beads” can produce functional polypeptides and/or putative binding partners and can be reused to synthesize additional polypeptides and/or putative binding partners. Furthermore, Ribo-beads may be used to enhance the optical detection sensitivity of putative binding protein and/or polypeptide synthesis and interactions by focusing polypeptide and/or putative binding partner translation activity to the bead surface. For example, novel lysates to be tested for increased protein production yields can be compatible with the Ribo-bead platform discussed herein. Use of the Ribo-beads may also speed up fluorescence correlation spectroscopy (FCS) interrogation by reducing the necessary scan volume to bead surfaces vs. bulk volume to detect molecular diffusion. In addition, synthesized proteins can accumulate near the beads, creating punctate regions that can enhance reporter signal enough to implement faster, non-FCS detection techniques (e.g., confocal laser scanning microscopy), further increasing assay throughput—critical to ensuring assaying 10³ to 10⁵ reactions within target goal. Ribo-beads can be localized inside microreactors or formed into packed beads to further increase fluorescent reporter concentration and intensity.

This technical solution can start with, for example, known scFvs against specific antigens as a test case. For example, this technical solution can utilize a previously generated polypeptide, LcrV, and can demonstrate interaction between LcrV and single chain antibodies as an initial validation of this approach. Cell-free lysates can be tested for total protein yield, solubility and purification yield. Techniques to evaluate total protein yield, solubility, and purification yield are well known in the art and those of ordinary skill could readily select and use such techniques in accordance with the methods of the present disclosure. Flourescent proteins, such as GFP or RFP, can be used as controls for comparison and benchmarking how well lysates perform alone, with Ribo-beads, or when encapsulated.

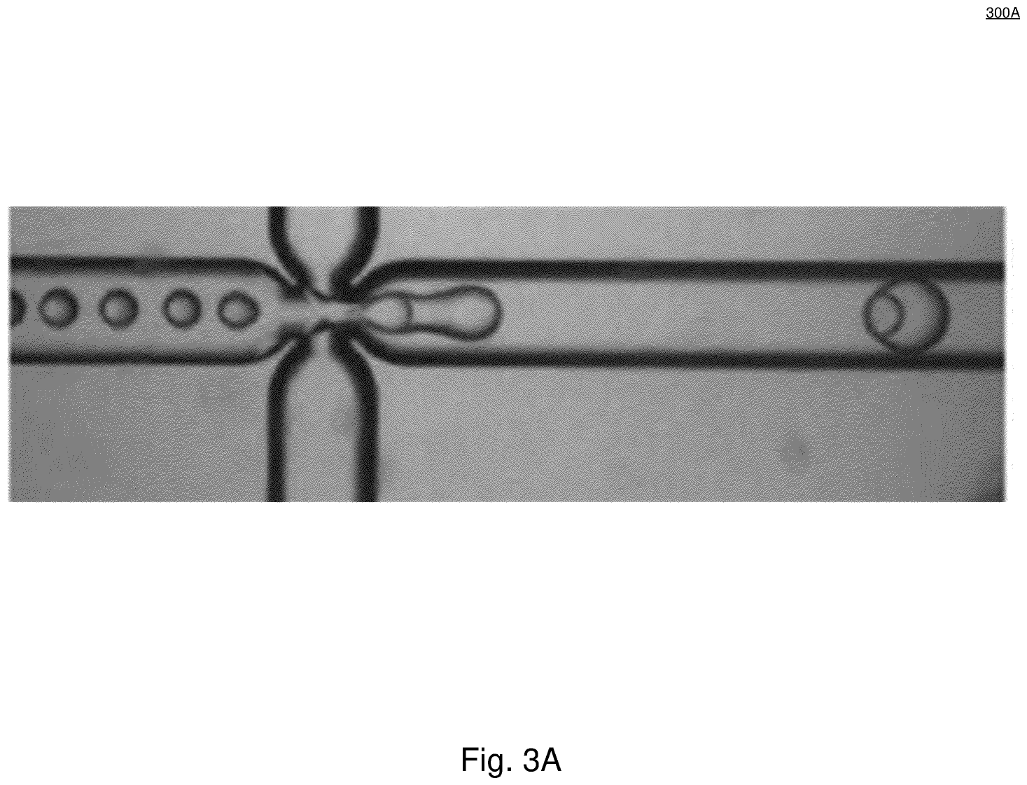

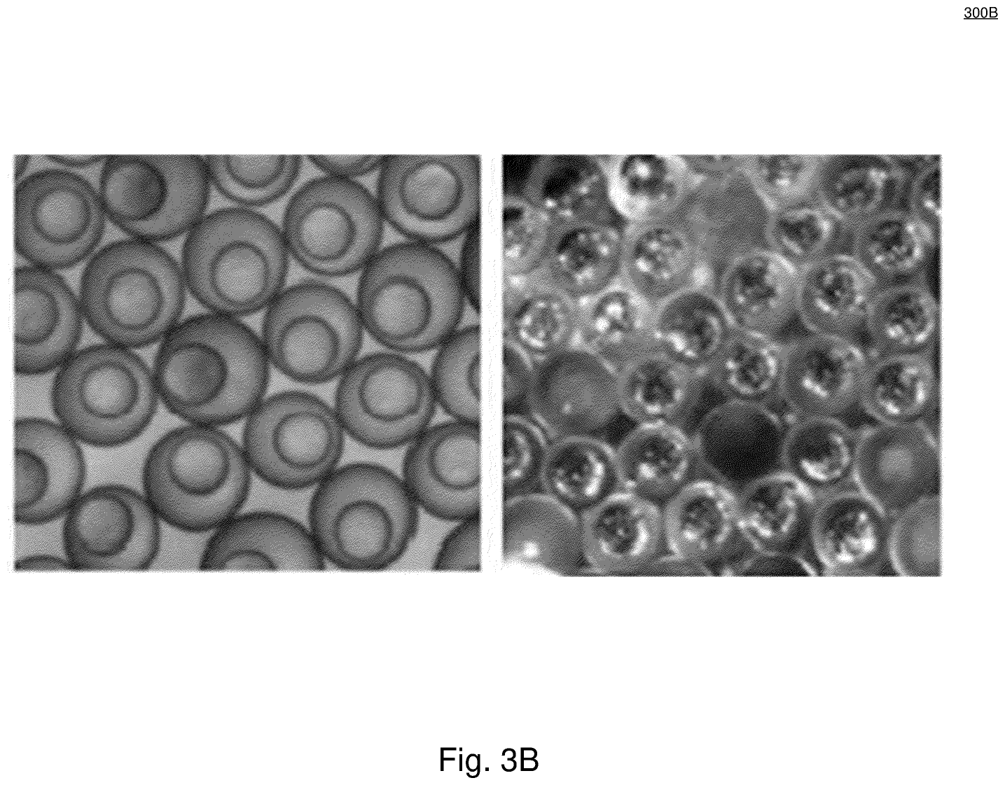

FIGS. 3A-B are directed to examples of microencapsulation according to this disclosure. FIG. 3A depicts an example encapsulation of cell-free lysates, according to this disclosure. FIG. 3B depicts example encapsulated cell-free lysates, according to this disclosure. For example, FIG. 3A shows W-O-W microcapsules being generated at the second microfluidic junction. and FIG. 3B shows microcapsules packing together while in storage. FIG. 3B shows the results of using the fluid handler to inject different dye samples that are partitioned into microcapsules via the complete loading method. In the right-side sample, the collected batch contains microcapsules with different core colors.

This technical solution includes microencapsulation development and integration for production of recombinant polypeptides (e.g., antibodies or antibody fragments). Various cell-free protein synthesis reactions may be carried out in, for example, a 96-384 well format, enabling one to potentially synthesize hundreds of pairs of polypeptides or putative binding partners at a time. This can be done in both batch and sustained production modes. However, to be able to analyze the full range of, for example, antibody mutations to further train (e.g., using supervised and/or unsupervised techniques) a machine learning system, this technical solution can analyze thousands of potential pairs. To facilitate high throughput characterization of, for example, antibody-antigen pairs, this technical solution can microencapsulate the cell-free protein synthesis, creating a microreactor. Each microreactor can contain the reagents necessary to synthesize copies of one antigen and one antibody, for example, using a green fluorescent protein label fused to either of the antigen or the antibody. By leveraging monodisperse droplet production capability of a microfluidic system, cell-free protein synthesis reactions can be carried out in the multilayer microcapsules of varying size from micrometers to millimeters with different thicknesses based on the polymer used to optimize the microcapsules for use in spectroscopy techniques. This solution can also generate 10³ microcapsules in a single run and this can further optimize to potentially achieve 10⁴-10⁵ microcapsules if needed by optimizing device setup and operation parameters. During production, immiscible fluids can be utilized in microfluidic devices to create emulsions which can be converted to microcapsules. Specifically, the DNA encoding one or more polypeptides and/or one or more putative binding partners in buffer with any additional additives can be mixed with the core fluid containing the cell-free protein synthesis reaction. This core fluid can be surrounded by a polymer shell layer that is co-generated to provide a controlled reaction for protein production in a single, encapsulated environment. The shell polymer can be cured using UV light to produce a solid microcapsule shell, providing a temporary compartment for facilitating measurements of specific antigen-epitope pairs. This system can be amenable to varying the concentrations of small molecules and/or antigens to create an environment for association and binding kinetics.

Multiple compartments can be created using liposomes to hold cell-free lysates and polynucleotides that then become encapsulated within the liposome. After cell-free protein synthesis, the shell materials of the compartment can be actuated and release the cargo for FCS studies. The specific actuation mechanism depends on the material choice, but can be accomplished via heat, pressure, and/or pH changes. Ribo-beads may also be useful to create zones of compartmentalization within the overall microreactor, which may also enhance spatial control of the cell-free protein synthesis reaction within the microreactor. These Ribo-beads may be used to limit protein translation activity to a specific area within the microreactor. In addition, this may enhance the optical detection sensitivity of protein synthesis and interactions between polypeptides and putative binding partners by providing distinct space for separating transcription and translation from spatial area needed for detection. Microcapsule shell material choice and microcapsule size can allow for fine tuning of the reaction steps and FCS signal strength optimization.

The current instrument for encapsulation requires >3 mL of starting material to achieve encapsulation. To reduce volumes, this microencapsulation approach (e.g., formation of microreactors) can also be integrated into a microfluidic card platform that can both assemble the reaction microreactors and later facilitate high throughput analysis. By leveraging the monodisperse droplet production capabilities of certain microfluidic systems, microencapsulated cell free protein synthesis reactions can be carried out in large numbers (10³-10⁵ unique polypeptides or putative binding partners in single encapsulation experiment).

For example, this technical solution can achieve kinetic characterization of polypeptide-putative binding partner interactions using fluorescence correlation spectroscopy and validation of virus neutralization for top candidates (e.g., polypeptides with relatively high binding affinity to putative binding partner), including development of FCS for microencapsulated affinity studies. Rapid characterization of polypeptide-putative binding partner interactions can use Fluorescent Correlation Spectroscopy (FCS) to characterize interactions. FCS is capable of measuring single molecular diffusion statistics directly in solution and provides in situ measurements for both qualitative and quantitative assessment of complex interactions in solution. An example application includes binding measurements of Y. pestis effector proteins LcrV and YopB embedded in a nanolipoprotein. This solution can use two colors to monitor interactions via FCS, in combination with alternating laser excitation (ALEX), which can allow for sensitive, quantitative detection of binding with low false positives. Each binding assay can be completed in 30 seconds to 5 minutes of acquisition per microreactor. Sensitive detector arrays, with up to 512 by 512 detectors, can be used for parallelizing the FCS technique to thousands of assays simultaneously. This technique is capable of assessing single molecule level interactions at a temporal and spatial level that has never before been fully realized and is highly useful for characterizing polypeptides, putative binding partners, protein complexes and individual protein structure dynamics. FCS can demonstrate that the instrumentation is capable of inferring topology and monodispersity for affinity bound complexes in a similar form to what has been accomplished for atomic force microscopy (AFM) technology. The technology can be integrated for use with a fluidic card to automate the imaging technology, facilitating high-throughput measurements within individual microreactors.

Specifically, this technical solution can design fusion proteins of appropriate putative binding partners (e.g., antigens) fused to a fluorescent protein (FP) as described elsewhere herein. These putative binding partner-FP proteins (e.g., antigen-FP proteins) can be co-expressed with polypeptides (e.g., full-length antibodies, scFVs) within cell-free protein synthesis reactions. For example, binding of scFvs to antigen-FPs can induce fluorescent spectral shifts that can be detected by FCS. This technical solution can also use FRET pairs to detect binding. In such an example, the scFv proteins can be fused to a donor FP (e.g., sfGFP) that makes a FRET pair with an acceptor FP (e.g., mTagRFP) fused to the antigen of interest. If binding occurs, excitation of the donor FP can result in fluorescence emission by the acceptor FP. Both FCS and FRET based fluorescence measurements can be compatible with this microencapsulation technology and automated imaging methods can be developed to make rapid and accurate measurements.

For example, this technical solution can include cell-based validation of affinity studies. Upon identification of top polypeptide (e.g., antibodies, BCMs) candidates that have been validated as having high affinity for their directed putative binding partner, this technical solution can evaluate them for the ability to, for example, neutralize their viral target. Specifically, antibodies (e.g., scFvs) can be preincubated with their target virus (e.g., comprising the target viral antigen that can be specifically bound by the scFv) across a range of concentrations to allow for binding. Preincubated virus can then be added to susceptible host cells (e.g., Vero cells) and growth of the virus can be assessed by, for example, standard plaque reduction neutralization test (PRNT). Antibody efficacy can be assessed as inhibition of viral growth, with more effective antibodies inhibiting viral growth at lower concentrations. Results from PRNT assays can be compared to results from the experimental assays described herein and can serve to validate this novel antibody production and validation methods.

Although examples of this technical solution are discussed using Ab-Ag interaction screening, it is modular and flexible enough to be expanded to many potential applications, including cell-based drug screening. High-throughput drug screening can be done using drug compound libraries or drug combinations tested on specific polypeptides (e.g., enzymes) or cell types of interest seeded on 384- and 1,536-well plates, with smaller volumes being favored to reduce reagent costs. Robotic liquid handlers and fluorescence-based detection methods can reduce the hands-on time required to perform these screens. With this platform, whole cells can be mixed with compounds of interest, encapsulated together within small volume microreactors, and incubated over time in a single step. Fluorescent readouts, either from genetically encoded reporters in the cells themselves, or DNA- or metabolism-based viability dyes, can be adapted into the platform and offer sensitive hit identification strategies. Direct protein or enzyme inhibition screening can also be performed by encapsulating purified or cell-free protein synthesis-produced proteins and assayed through inhibition of fluorescence resonance energy transfer (FRET) signals such as for the SARS-COV-2 spike protein and the hACE2 receptor.

Directed evolution of proteins can also be facilitated with this microfluidic platform. Evolving proteins, especially enzymes, for increased activity or stability requires large library sizes of mutants, typically generated through error-prone polymerase chain reaction (PCR), and iterative rounds of functional screening in cells. This platform can handle not only random mutagenesis libraries but also rationally designed libraries as inputs to be expressed using cell-free protein synthesis lysates. Screening for an optimized nuclease can present a hypothetical application, as a quenched DNA probe containing the targeted cleavage sequence can be included directly into the cell-free protein synthesis core fluid mix. As the nucleases are produced and cleave their targets after encapsulation, the quencher is released, facilitating a fluorescent optical readout. The microreactors rising above a certain threshold can be separated and sequenced to discover the mutations leading to increased activity. Because the shell phase of this droplet generation system is biocompatible, the CFPS core fluid can also be swapped out to include live cells in the case of a directed evolution approach, or if cell viability is a required functional readout, while still allowing for high throughput droplet generation. While these are use cases for this platform, this solution can achieve increased flexibility and scalability to enable and facilitate high-throughput screening in general.

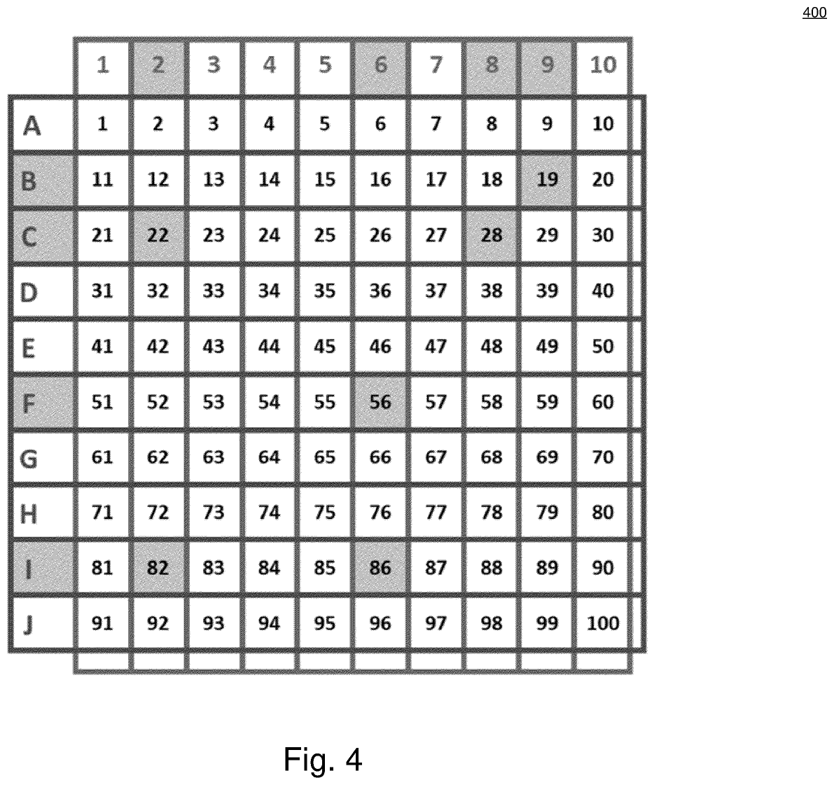

FIG. 4 depicts example candidate samples, according to this disclosure. As illustrated by way of example in FIG. 4, blue rows are ten pools, each comprising ten Ab candidates with unique identifier numbers. Orange columns are also ten pools that contain candidates that are shared with the blue pools. Green fill denotes a positive outcome (Ab candidate bound with antigen with a relative high binding affinity) for the pool and the individual candidate, that latter of which follow-on tests are needed to decipher.

Partitioning samples into microreactors rapidly creates thousands of independent reactions that are run in parallel. Pooling multiple polypeptides and/or putative binding proteins into individual microreactors can drastically increase throughput by reducing the maximum number of tests needed to detect positive outcomes. FIG. 4 illustrates two-dimensional pooling of 100 polypeptides (e.g., Ab candidates binding to a target viral spike protein) as a simple example. The 100 antibodies are organized in a 10×10 array and are pooled into twenty different batches based on the array's rows and columns. This ensures that each candidate is in at least two pools, which is important in order to ascertain its activity. Each of the twenty pools are tested for activity against the viral Ag, with 8 total pools reporting positive outcomes (green). After only twenty tests, 84 candidates are eliminated. Follow-on testing of the intersections of each positive row with each positive column (16 tests) finds the five best performing candidates from a total of 36 tests compared to the 100 tests required using the individual testing (or 1:1) method. Pooling can be expanded to n-dimensions, greatly improving throughput. For example, only 258 pools and a total of 282 tests (258 initial tests with 24 verifications) can be used to screen 72,000 polypeptides. For example, the rate of positive outcomes (e.g., relatively high binding affinity) can be <20% (e.g., sparse matrices). Thus, this solution includes an automated, high-throughput method and platform to provide rapid experimental feedback to in silico Ab designs. This process creates microcapsules with rigid polymer shells that are interrogated with fluorescence correlation spectroscopy (FCS) to quantify the success of binding of the Ab to known Ag, such as viral spike proteins. The following details the process, software, and hardware implemented to reduce this embodiment to practice.

An example method of feedback is discussed below. A system according to this disclosure can comprise one or more of a data processing system (e.g., a computing system) and a robotic system. The data processing system can at least partially include or can execute a machine learning system as discussed herein. The data processing system generate output including one or more instructions to operate the robotic system, or to cause the robotic system to perform one or more actions. For example, the data processing system can identify one or more pools corresponding to output indicative of one or more predicted amino acid sequences of a polypeptide as discussed herein. For example, the data processing system, in response to receiving or generating output indicative of one or more predicted amino acid sequences of a polypeptide, instructs the robotic device to select a set of samples including a given presence of the one or more predicted amino acid sequences of the polypeptide as indicated by the output. The instruction to select can occur multiple times, as multiple iterations. For example, iteration includes an action to select a set of samples and to transfer the set of samples to an interrogation chamber. For example, each iteration is in response to a distinct output indicative of one or more predicted amino acid sequences preceding that iteration. The given presence can correspond to a number, percent, or other degree of presence of the samples in a set of samples to be selected. For example, the given presence is a minimum percentage of samples collected during an instance of operation of a robotic system. For example, the given presence is a minimum number of samples collected during an instance of operation of a robotic system. Thus, the system can iterate more rapidly toward generating amino acid sequences by a robotic device that is caused to provide samples that include a higher prevalence or presence of output that satisfy the criteria (e.g., threshold binding affinity) of the machine learning model of the system or the data processing system as discussed herein.

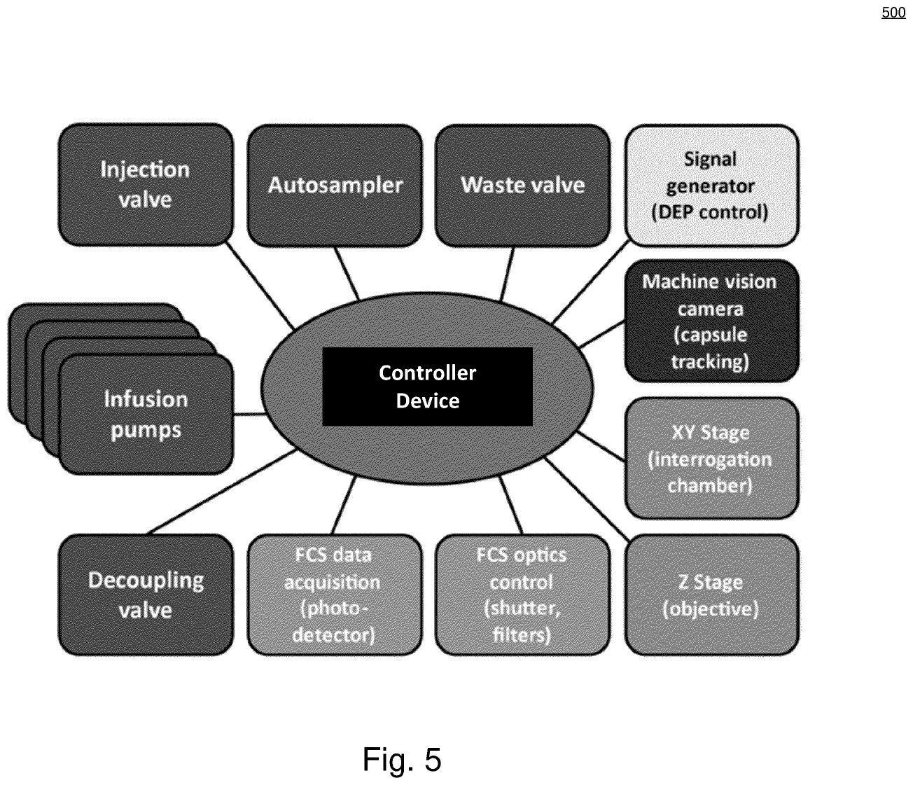

FIG. 5 depicts an example controller architecture, according to this disclosure. For example, FIG. 2 illustrates the process flow and hardware, and FIG. 5 illustrates a program architecture that controls and reads hardware to facilitate the automation process. For example, a fluid robot creates samples by mixing cell-free protein synthesis (CFPS) components with plasmid(s) that encode polypeptide and/or putative binding partner candidates that are being evaluated. Mixed samples may contain plasmids that define the putative binding partner, or alternatively may contain the putative binding partner. Samples may also contain plasmid(s) that encode multiple polypeptides and/or putative binding partners in the case the pooled screening technique is used. The robot may cool some samples to slow down or halt reactions until they are partitioned into microreactors. The robot injects each sample into an injection valve, completely filling its integrated sample loop. The valve is then actuated to allow a pressure pump filled with immiscible fluid to force the loaded sample out of the loop and into the core inlet of the encapsulator chip. Another pump infuses the TEGO Rad shell solution to encapsulate the aqueous sample at a microfluidic junction, creating water-in-oil (W-O) microdroplets. A glycerol-based carrier solution is pumped into the chip to encapsulate the W-O microdroplets at a second junction to create W-O-W microcapsules that exit the chip for downstream processing.

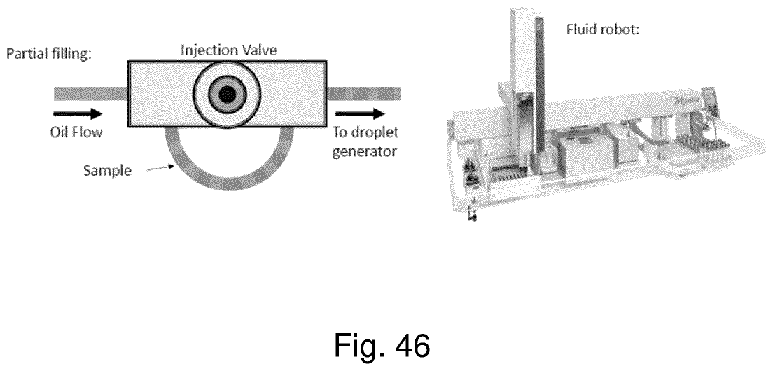

The injection valve's loop may be filled completely with a single sample (i.e., complete filling), or it may be filled with multiple injections of different samples of lesser volume. This latter method, known as partial loading, enables the platform to generate multiple microreactor payloads per sample loop purge cycle, which can increase sample throughput and decrease sample volume requirements (FIG. 46). Each of the injected samples can be separated by the water-immiscible phase to ensure no cross-contamination. These oil partitions within the sample loop create O-W microdroplets once they exit the encapsulator chip and can be distinguished from the W-O-W, sample-filled microcapsules. The immiscible fluid used to separate individual samples can be the same solution as the middle oil phase, or an alternate fluid that is immiscible with the sample phase as is necessary for operation. For example, TEGO Rad is considerably viscous and limits the speed at which samples can be injected into the sample coil due to the limited pressure head available to standard liquid handlers. Thus, less viscous water-immiscible fluids such as silicone oil may be used in place of the TEGO Rad solution to reduce the hydraulic resistance and enable more rapid sample delivery. Partial loading can be completed using a fluid-handling robot (“fluid robot”). In some embodiments, use of partial loading can dispense about a 0.1 μL sample volume, about a 0.2 μL sample volume, about a 0.3 μL sample volume, about a 0.4 μL sample volume, about a 0.5 μL sample volume, about a 0.6 μL sample volume, about a 0.7 μL sample volume, about a 0.8 μL sample volume, about a 0.9 μL sample volume, about a 1 μL sample volume, about a 1.2 μL sample volume, about a 1.5 μL sample volume, about a 1.8 μL sample volume, about a 2 μL sample volume, about a 3 μL sample volume, about a 5 μL sample volume, about a 8 μL sample volume, or about a 10 μL sample volume into the sample loop. Accordingly, such multiple injections of different samples of lesser volume can improve throughput by evaluating about 5, about 8, about 10, about 12, about 15, or about 20 samples (e.g., microreactor payloads) per sample loop purge cycle.

In methods of the present disclosure, oxygen levels within the system and/or microreactor according to the disclosure can be measured, managed, and/or controlled (e.g., such that expressed polypeptides are fully functional). One of ordinary skill in the art can readily determine whether or not an expressed polypeptide is fully functional using techniques known in the art. Without wishing to be bound by any one theory, it is understood that variation in FCS-measurement intensity can be a result, at least in part, of dependency of GFP activity on microreactor position within the system, there being (i) higher signal for microreactors near the edge of the interrogation chamber (e.g., due to access to air/oxygen through a gasket of the system) and near “spacer” microreactors that form the partitions between sample microreactors (e.g., due to spacer microreactors containing unexpired oxygen) and there being (ii) lower signal for microreactors within the center of their respective group in the interrogation chamber (e.g., due to being away from oxygen sources). Thus, in some embodiments, oxygen levels within a system and/or microreactor of the present disclosure can be measured, managed, and/or controlled. Oxygen levels can be measured using techniques known in the art, for example, by titration methods, electrochemical analyses (e.g., diaphragm electrode method), and photochemical analyses (e.g., fluorescence methods). In some embodiments, oxygen levels are managed and/or controlled by utilizing pressure pumps to drive fluids comprising oxygen into the system prior to microreactor generation. In some embodiments, oxygen levels are managed and/or controlled by agitating and/or infusing fluids comprising oxygen or air in-line between pumps, but prior to microreactor generation. In some embodiments, oxygen levels are managed and/or controlled by passing generated microreactors through an oxygen diffusion region before reaching the interrogation chamber. In some embodiments, oxygen levels are managed and/or controlled by infusing air and/or oxygen into the chamber that comprises the microreactors. In some embodiments, oxygen levels are managed and/or controlled by using oxygen gas to pressurize a system of the present disclosure (e.g., via a pressure pump), thereby creating flow through the system. Without wishing to be bound by any one theory, it is understood that the oxygen gas will diffuse into fluids utilized in a system of the present disclosure to provide additional oxygen to the microdroplets. In some embodiments, one or more of the aforementioned oxygen measurement and/or management/control methods are utilized in accordance with technologies of the present disclosure.

After exiting the encapsulator chip, the ˜400 μm microcapsules flow through an illumination region that uses high-intensity ultraviolet radiation to rapidly crosslink their TEGO Rad shells within seconds, creating solid microcapsules with liquid cores. The solid shells create microcapsules that are mechanically robust, preserve their contents for many months, and can not re-coalesce with other microcapsules, regardless of duration. The crosslinked microcapsules reach the decoupling valve, which has the main function of decoupling the high flow outlet of the encapsulator chip (˜200 μL/min) from the interrogation chamber. Another important function of this valve is it provides control over what portion of generated microcapsules are kept for FCS analysis, instead of allowing microcapsules of poorer quality to make it to the interrogation chamber. This valve is a two-position, six-port valve that either sends the fluid stream from the encapsulator chip to waste or into an integrated holding coil. If the microcapsules are permitted to continue toward the interrogation chamber, the decoupling valve is actuated to load them into the holding coil. Once the coil is filled with the desired amount of microcapsules, the valve is switched to allow a fourth pressure pump to infuse a glycerol carrier solution through the coil to push the microcapsules toward the waste valve. The waste valve is a sample selector valve that is used to either allow microcapsules to be transported from the decoupling valve loop to the interrogation chamber, or to purge the coil from all microcapsules and contaminants to waste before the next cohort of microcapsules arrive.

For example, microcapsules enter the ˜500 μm-high interrogation chamber, which rapidly expands in width to decrease fluid and microcapsule velocity. For example, crosslinked microcapsules flow into the chamber. This decrease in velocity is important, as it enables proper microcapsule tracking without taxing computational resources and reduces the required forces needed to hold back the microcapsules at the exit gate of the chamber, which can be described shortly. For example, the microcapsules populate the chamber in a single layer to ensure accurate tracking and analysis. Therefore, the chamber height needs to be no more than twice the nominal diameter of the microcapsules. Given this criterion, the increase in width is also important in that it increases throughput by allowing more microcapsules to be viewed, tracked, and analyzed at once. For example, microcapsules are tracked upon entering the interrogation chamber and continue to be tracked until they are permitted to exit the chamber after FCS analysis. A custom Python algorithm identifies trackable entities via a machine vision camera and lens, providing them unique identifiers and attaches X, Y coordinates and other relevant data to them for every time point. Knowing the real-time centroid coordinates of each microcapsule is advantageous for accurate FCS analysis. The XY stage is moved to that particular set of coordinates to ensure the FCS objective is centered within the core of the capsule that it is analyzing, which is advantageous for obtaining time series data for a plurality of microcapsules. Herein, “capsules” and “microcapsules” can be used interchangeably.

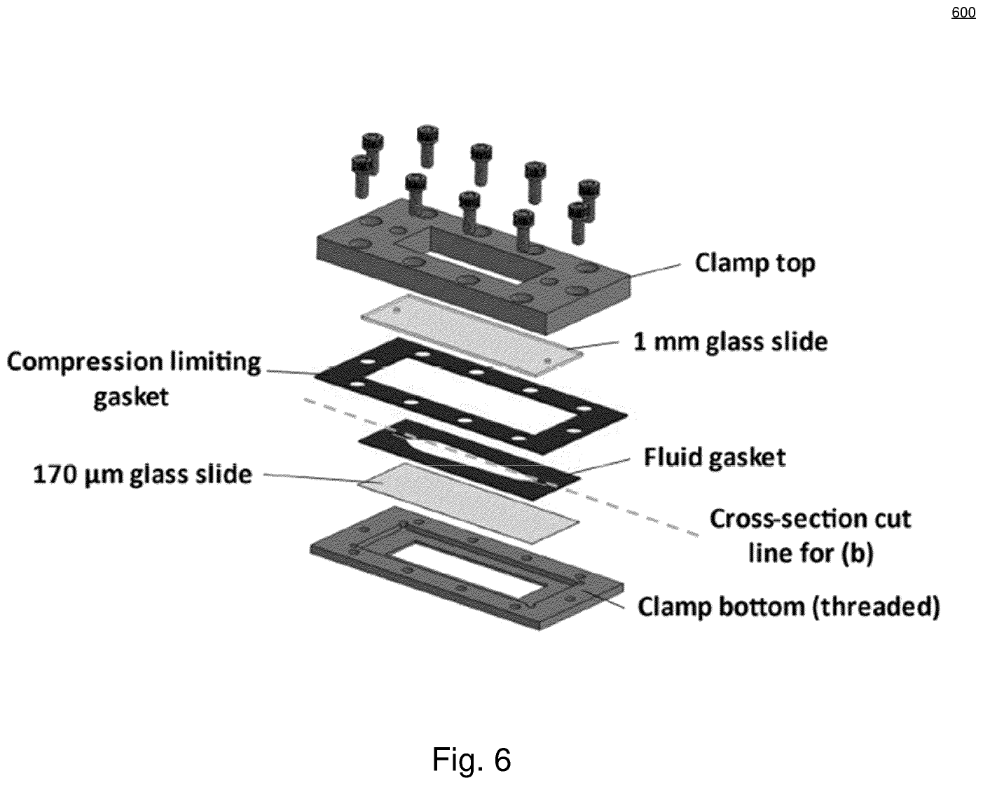

FIG. 6 depicts an exploded view of an interrogation chamber, according to this disclosure. For example, electrodes are integrated into the chamber via conductive tape to create a DEP exit gate that can hold the microreactors within the chamber while they are being loaded or undergoing analysis. The electrodes span the width of the entire chamber and are placed orthogonal to the fluid flow direction. The electrodes are oriented vertically to ensure the electric field spans the height of the channel, to prevent microcapsules from “hopping” over the fields on their way to the exit. The electrodes are preferred to be on the outside of the chamber to avoid fouling from direct interaction with the fluid. However, the thickness of the top slide reduces the field intensity substantially, requiring the top electrode to be placed within the chamber. A 50 MHz, 1 VRMS voltage signal from a signal generator is amplified 45 dB before being injected across the electrodes. At this drive frequency, the polymer shells of microcapsules are less polarized than the glycerol carrier solution in the non-uniform electric field. This creates a negative DEP force on the microcapsules, pushing them away from the high field regions near the edges along the length of the electrodes. As long as the fluid drag force on the microcapsules (depends on velocity and viscosity of fluid) does not exceed that of the DEP force, microcapsules can be prohibited from passing on to exit the chamber; additional microcapsules may continue to be loaded into the chamber and/or carrier fluids may be exchanged.

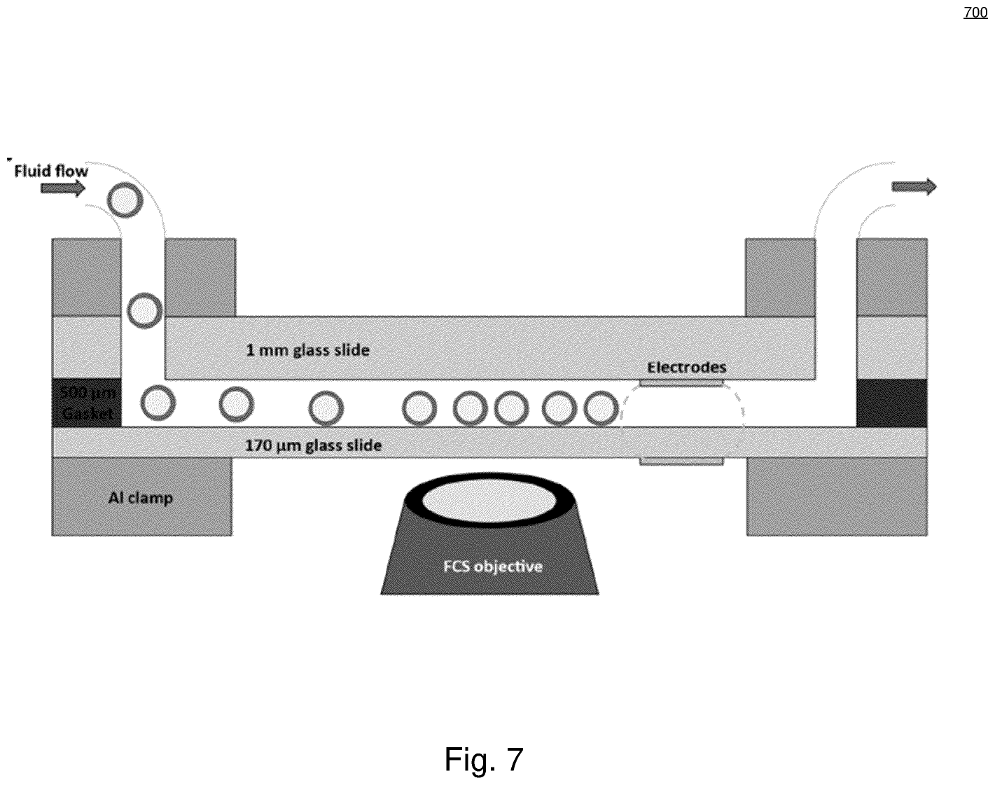

FIG. 7 depicts an interrogation chamber with microcapsules, according to this disclosure. FIG. 7 illustrates how negative DEP is successful in preventing polymerized microcapsules from passing the electrodes in a glycerol carrier solution. A combination of a different carrier fluid, drive frequency, and microcapsule shell composition may result in positive DEP forces acting upon the microcapsules. This also creates a gate effect, as the microcapsules can pin at the high field regions along the electrode edges, preventing aft microcapsules from passing. The geometry and configuration of the electrodes may be altered to enhance DEP forces, including, but not limited to, interdigitating them on the X-Y plane (plane that width and length of chamber lie on); offsetting the electrodes in the X direction (length axis of chamber) so that the outer edge of one aligns with the inner edge of the other; and placing both electrodes inside the chamber to enhance field strength. Electrodes may be formed using other methods than conductive tape: standard microfabrication methods on glass (metal evaporation followed by lift-off or etching techniques); wire; conductive inks, pastes, and paints; and foils fused to the surface of the glass slides.

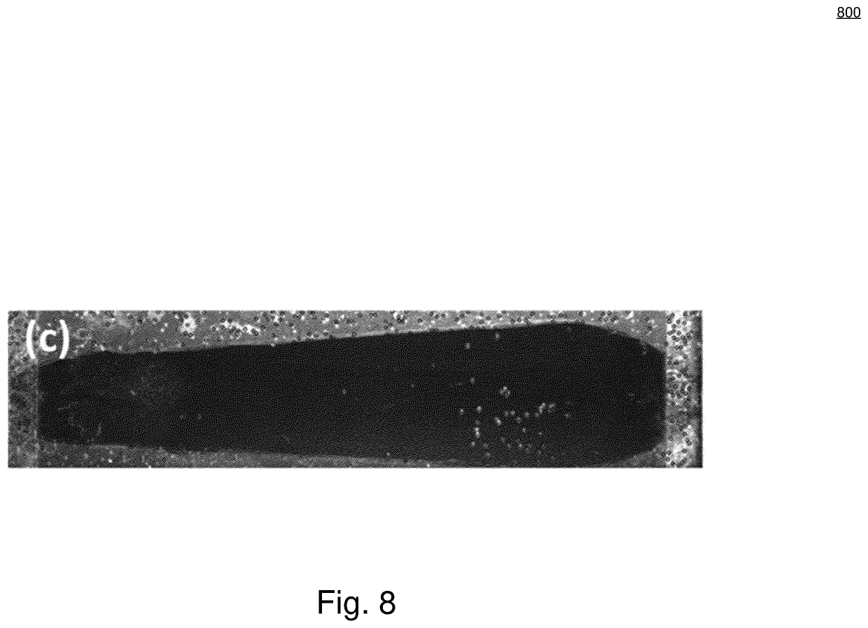

FIG. 8 depicts a plan view of microcapsules in an interrogation chamber, according to this disclosure. FCS measures the diffusion biophysics of fluorescently labeled molecules (e.g., polypeptides, putative binding partners) in solution. A system according to this disclosure includes an excitation laser beam focused through an objective into the sample solution, a confocal pinhole, and single photon detectors that capture the emitted fluorescence. FCS has the sensitivity to observe single molecules diffusing through the focal volume. This approach uses FCS to quantify affinity interactions between polypeptides and putative binding partners within microcapsules by observing the diffusion of fluorescently-tagged polypeptides and/or putative binding partners. For example, fluorescently labeled SARS-COV-2 spike protein and different candidate Ab plasmids are encapsulated within each microcapsule and measured by FCS. The antibodies are produced inside the microcapsules by cell-free protein synthesis. The fluorescence signal is processed to yield the correlation curve describing the diffusion characteristics of the fluorescently labeled molecules. Promising Ab candidates with high affinity, for example, to the spike protein can produce a rightward shift in the correlation curve as the larger protein-Ab complex diffuses more slowly. This is the readout of a positive hit. The use of FCS inside microcapsules may be generalized to screen binding between other biomolecules.

This technical solution thus achieves a technical improvement at least to perform FCS measurements of polypeptides and/or putative binding partners produced inside microcapsules by cell-free protein synthesis without the need for purification. This technical solution successfully demonstrates the former, as shown in the Examples provided herein, by encapsulating cell-free lysate and GFP plasmid, then measuring FCS inside the microcapsules over time. Over the course of several hours as GFP is produced, the correlation curves decrease in amplitude since amplitude is inversely proportional to concentration. The measured diffusion times (˜350 microseconds) stay constant over time.

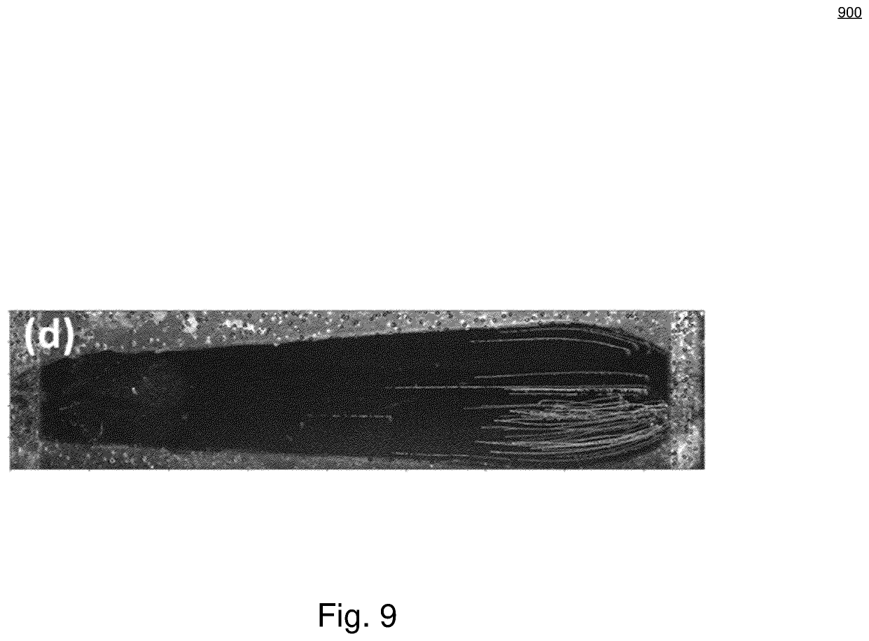

FIG. 9 depicts a plan view of tracked movement of microcapsules in an interrogation chamber, according to this disclosure. This solution thus achieves a technical improvement at least to obtain the binding affinity between polypeptide and putative binding partner using a time course of FCS measurements, for example, by measuring FCS over time in a microcapsule containing an anti-GFP Ab, GFP plasmid, and cell-free lysate. This system obtains the complete curve by a time series of one sample of cell-free protein synthesis reaction. In this example, the overall diffusion time of GFP is initially high because most of the small amount of GFP is bound to the Ab to form a larger diffusing complex. As the reaction generates more GFP, GFP saturates Ab binding, leaving increasing amounts of it to be free, thus decreasing the overall diffusion time to be that of free GFPs. This technical solution may process this data to obtain the fraction bound as a function of GFP concentration, to which a binding curve may be fitted to determine the binding affinity. This method produces a complete binding curve by FCS measurements on one sample solution within a few hours of sample preparation. After FCS analysis is complete, the DEP gate is disengaged, fluid flow increases, and the analyzed microcapsules are washed out through the exit port of the interrogation chamber into a tube for storage and subsequent analysis.

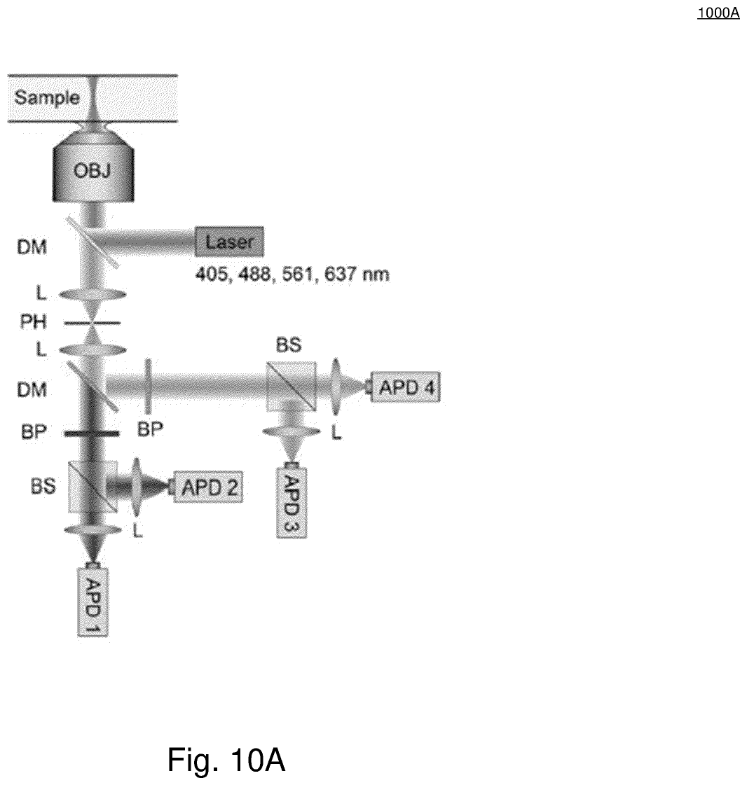

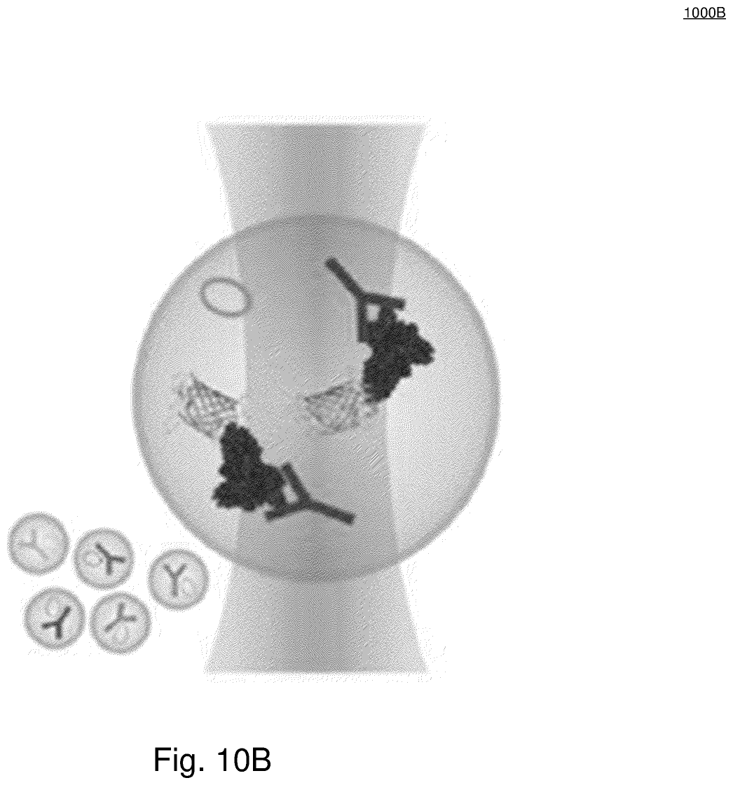

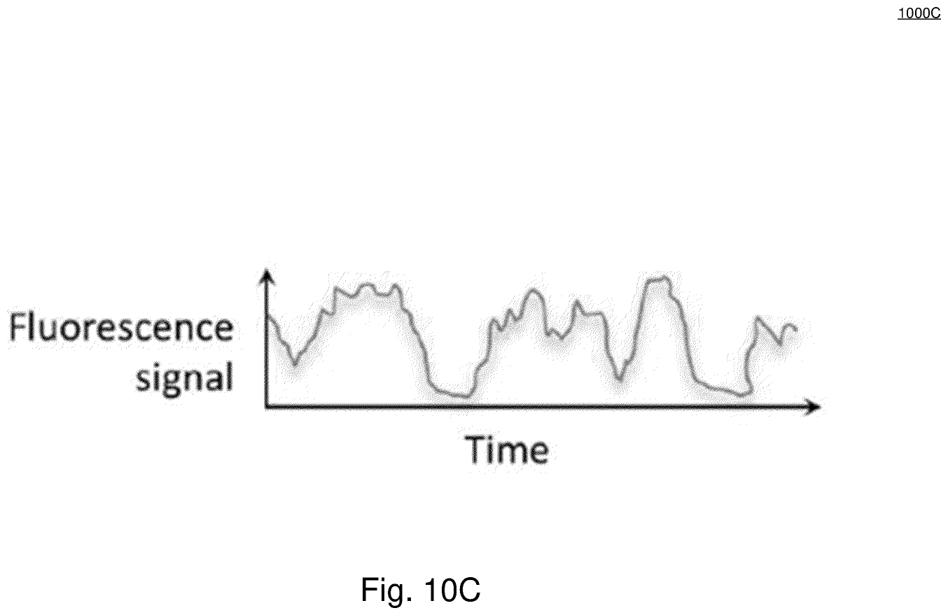

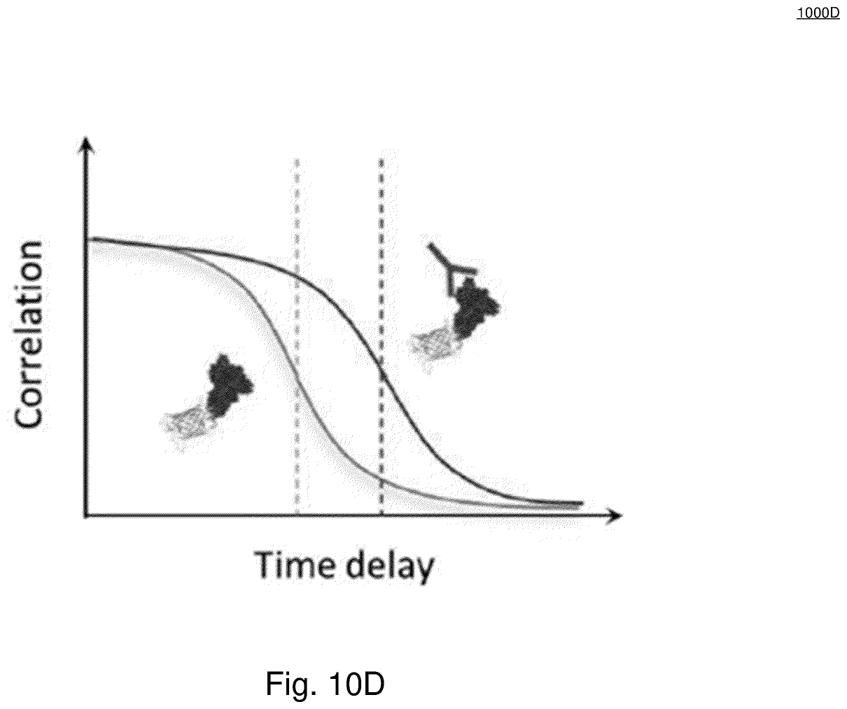

FIG. 10A depicts screening instrumentation, according to this disclosure. For example, FCS instrumentation is used for screening polypeptide binding to putative binding partners. FCS instrument schematic for this dual-color system based on a confocal microscope. The excitation laser is focused through the objective into the sample solution. Emitted photons from fluorescent molecules diffusing through the focal volume are captured by detectors which record the arrival times of each photon. The femtoliter-scale detection volume is defined by the excitation laser focus and the confocal pinhole. This system has the capability to use several different excitation lasers and emission wavelengths, enabling measurements of multiple species labeled with different fluorescent probes. OBJ: objective; DM: dichoric mirror; L: lens; PH: pinhole; BP: bandpass filter; APD: avalanche photodiode.

FIG. 10B depicts an example of a microreactor according to this disclosure. For example, each microreactor is an individual reaction chamber containing the proteins of interest (e.g., polypeptide, putative binder partner). In this example, each microreactor has the SARS-COV-2 spike protein labeled with the green fluorescent protein (GFP) and a different candidate antibody, which is produced inside the microreactor by cell-free protein synthesis using the template plasmid. The excitation laser is focused into the microreactor to measure the fluorescent molecules.

FIG. 10C depicts a fluorescence signal from microreactors, according to this disclosure. The fluorescence signal (photons detected by APDs) fluctuates over time as fluorescent molecules move through the focus. The second order correlation (g(2)) is calculated from the signal.

FIG. 10D depicts correlation curves from microreactors, according to this disclosure. For example, if a candidate Ab successfully binds to the spike protein (Ag), the correlation curve shifts to the right because the larger protein-Ab complex diffuses more slowly than the spike protein-GFP alone. This use of FCS inside microreactors may be generalized to screen binding between other molecules, such as polypeptides and small molecules.

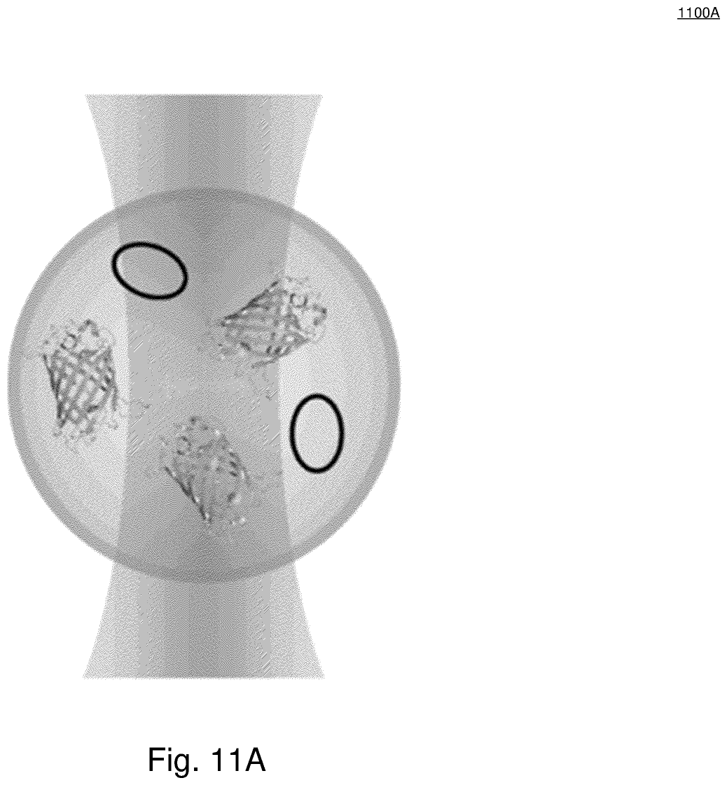

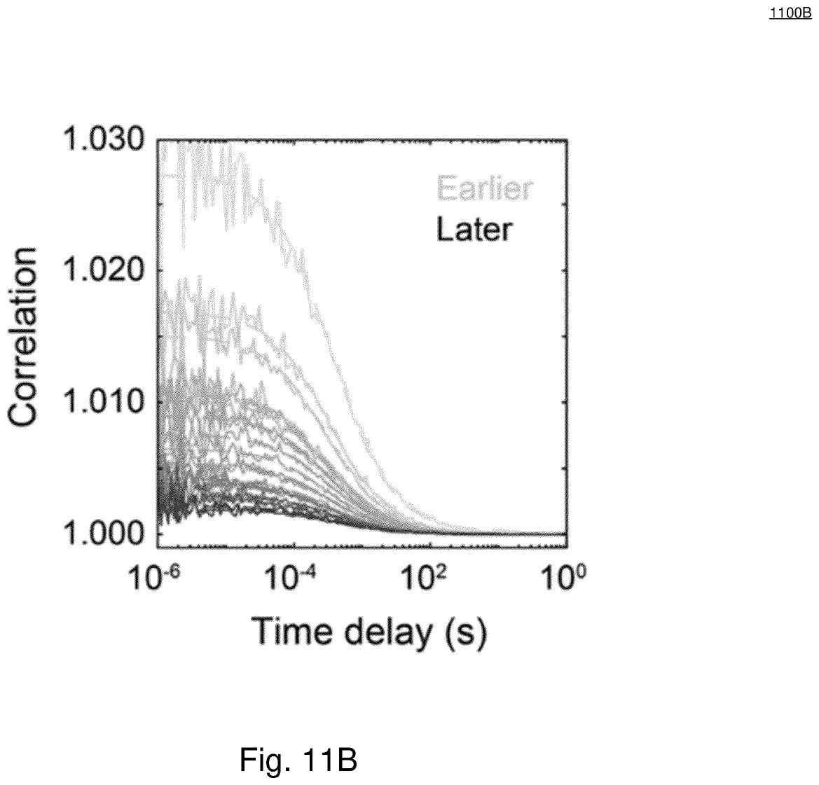

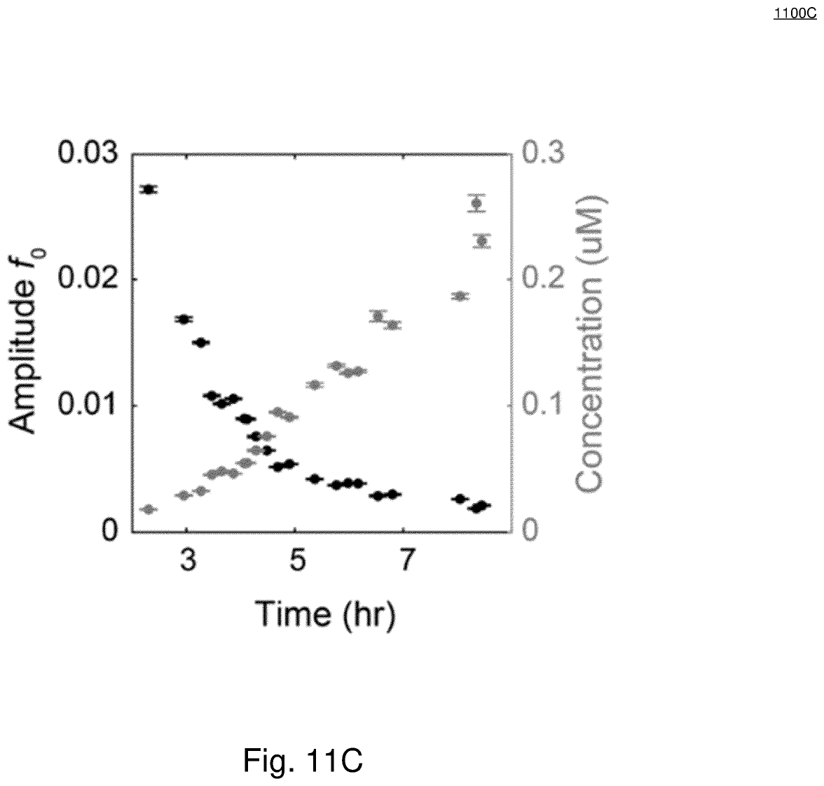

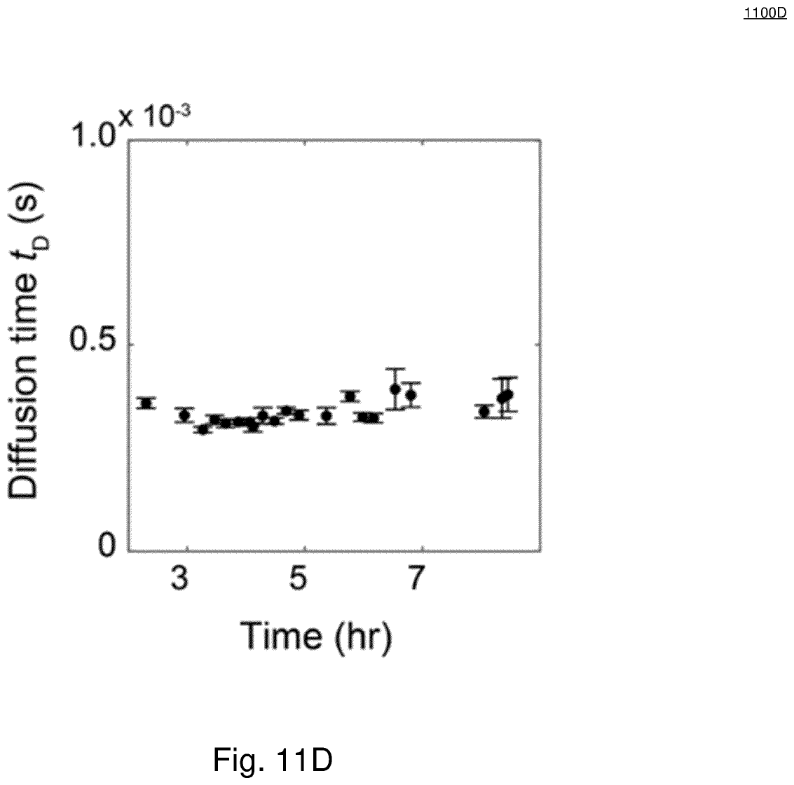

FIG. 11A depicts polypeptide produced inside microreactors, according to this disclosure. For example, FCS measures polypeptide produced inside microreactors in real time without the need for post purification. To demonstrate feasibility, this technical solution encapsulated plasmid encoding GFP and cell-free protein synthesis lysate and measured the production of GFP over time using FCS.

FIG. 11B depicts correlation curves from microcapsules, according to this disclosure. For example, FIG. 11B illustrates correlation curves of all time points, where shades of green denote the time progression.

FIG. 11C depicts amplitudes of correlation curves from microcapsules, according to this disclosure. Here, amplitudes obtained from fits to the correlation curves are plotted over experimental time (black). The measured concentration of the fluorescent molecule can be calculated from the amplitude, and is inversely proportional to the amplitude and increases over time as GFP is produced, as expected (green).

FIG. 11D depicts diffusion times from microcapsules, according to this disclosure. Here, diffusion times obtained from fits to the correlation curves are plotted over experimental time. GFP has a sub-millisecond diffusion time which stays constant over time, as expected. Data from this figure demonstrates the ability of this system to measure in situ the diffusion biophysics of protein produced inside microcapsules by cell-free protein synthesis, in the background of the cell-free lysate without the need for purification.

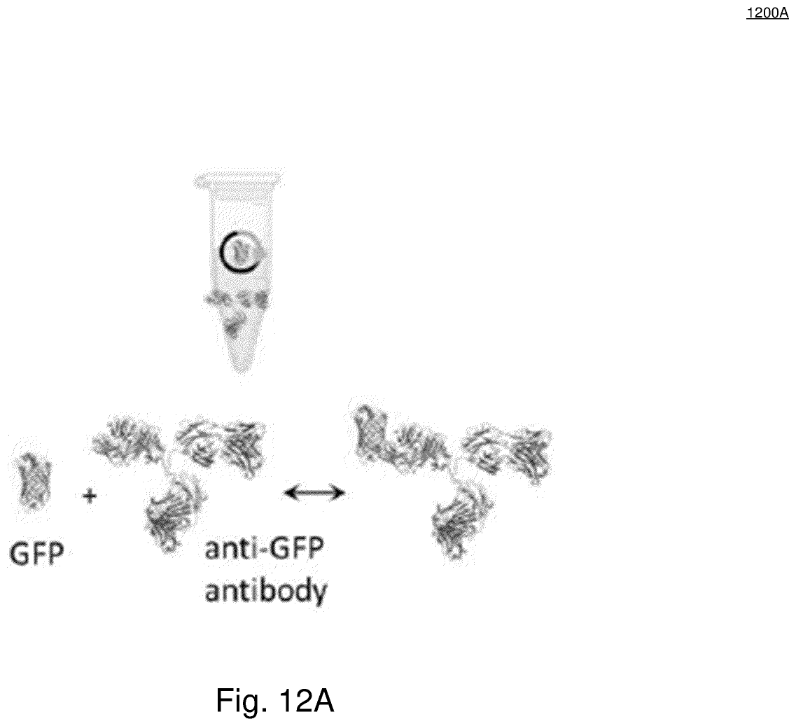

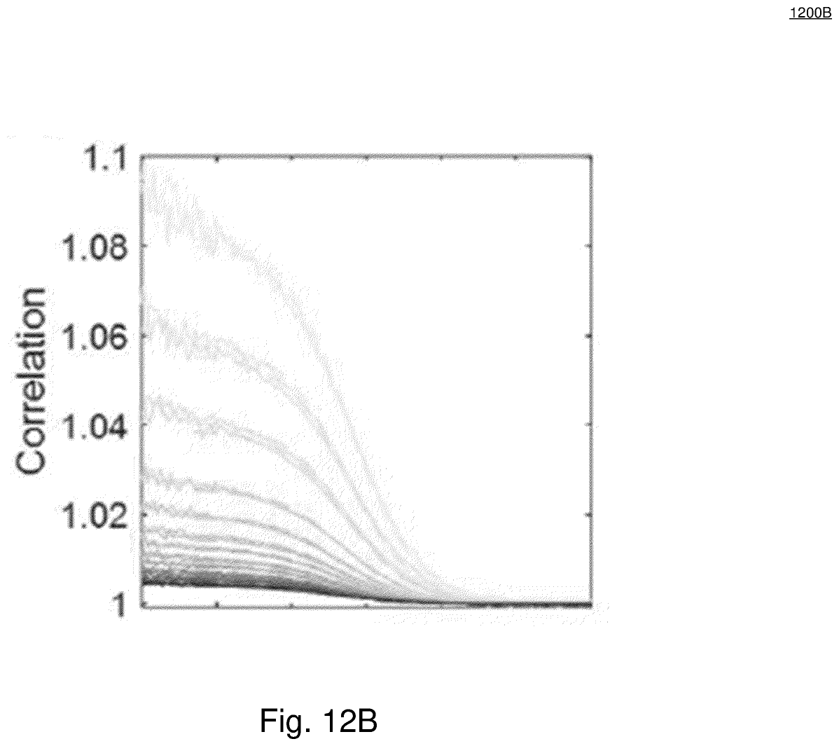

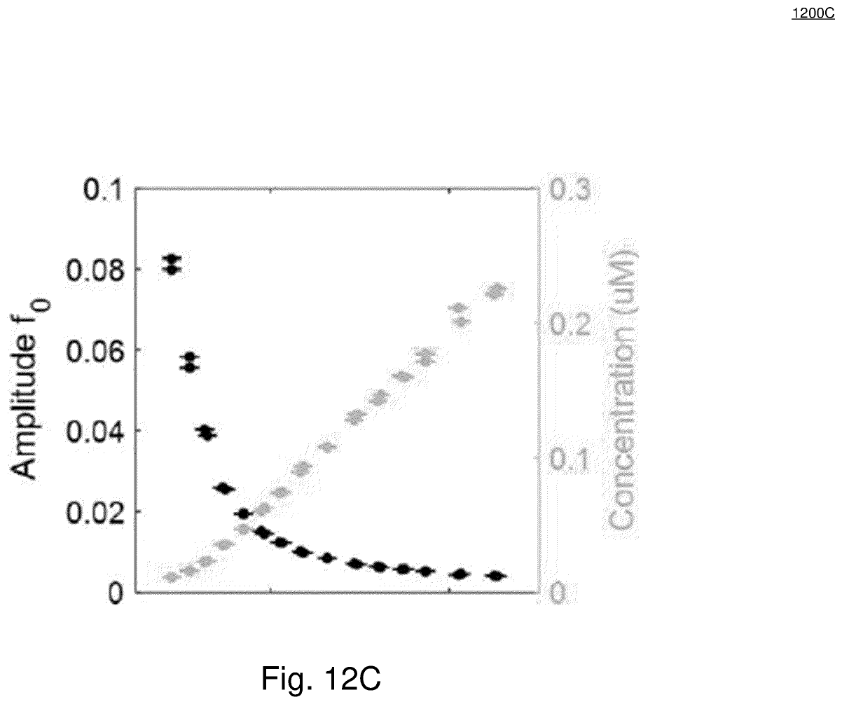

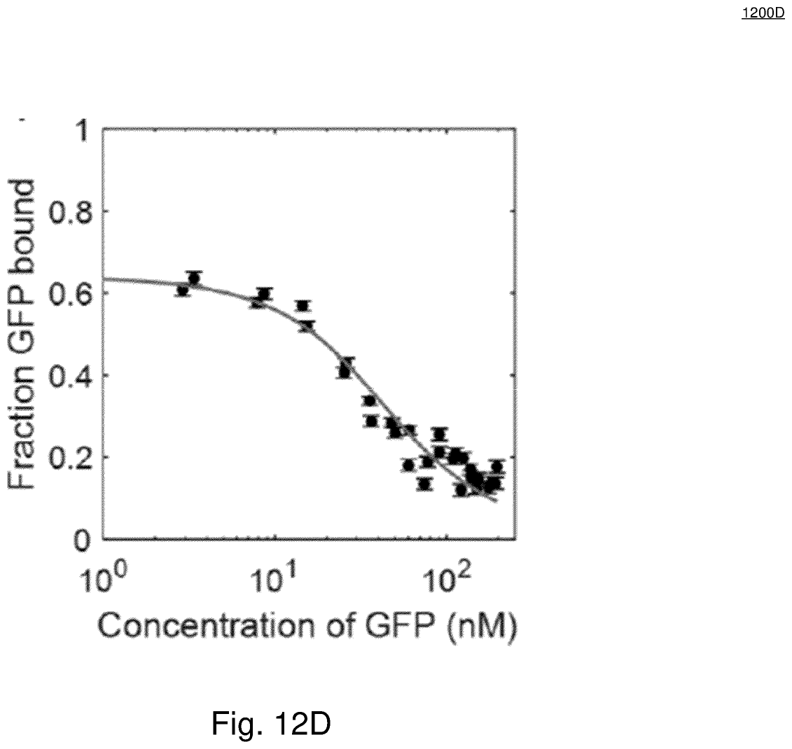

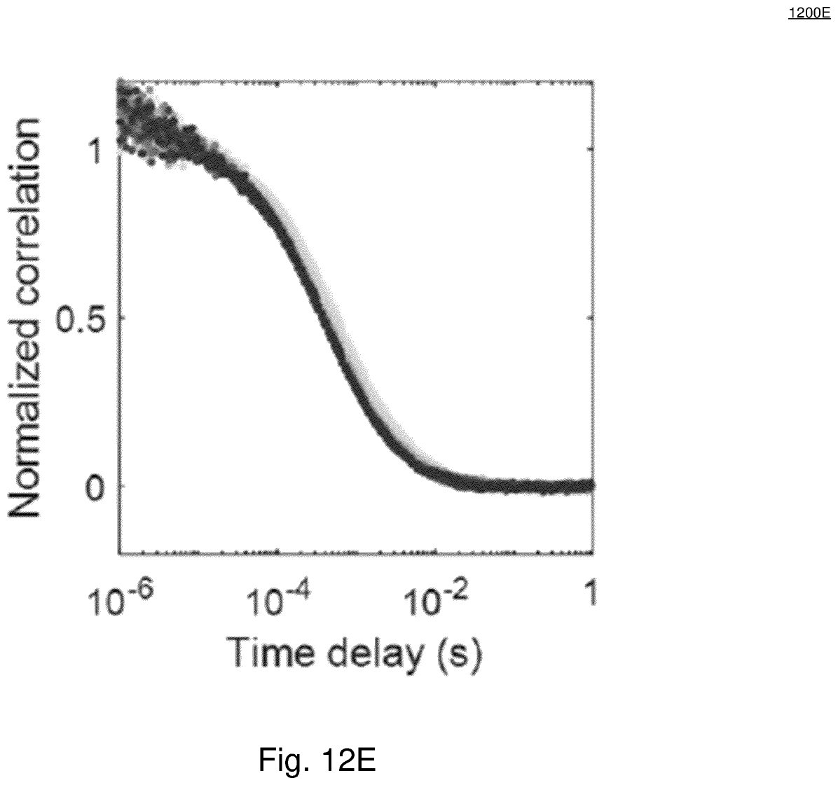

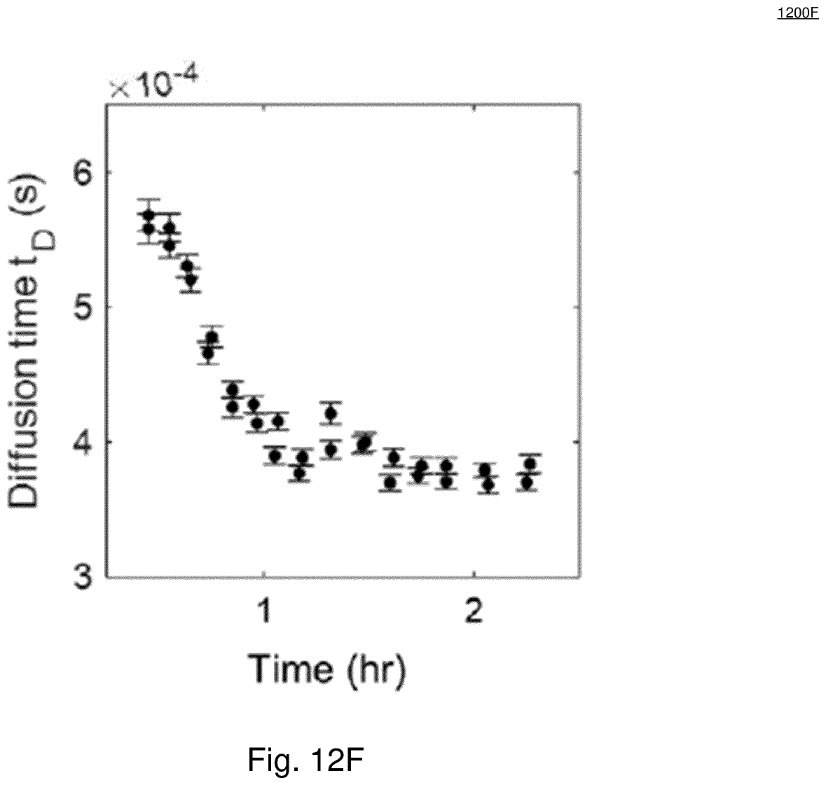

FIGS. 12A-D are directed to determination of binding affinity by FCS measurements of cell-free expressed proteins over time. FIG. 12A depicts protein produced inside microreactors, according to this disclosure. FIG. 12A illustrates one sample solution to obtain a complete binding curve, in contrast to methods which requires multiple solutions containing different concentrations of purified proteins. In this example, this solution measures the binding affinity of GFP to an anti-GFP Ab. The sample solution contains a set amount of the Ab, GFP plasmid, and cell-free synthesis lysate. FCS measurement of this solution are performed over several hours.

FIG. 12B depicts correlation curves from microcapsules, according to this disclosure. Here, FCS correlation curves of all time points are illustrated, where shades of green denote the time progression. The leftward shift is apparent in the plot of normalized correlations.

FIG. 12C depicts amplitudes of correlation curves from microcapsules, according to this disclosure. Amplitudes (black) and calculated concentrations (green) obtained from 1-species fits to the correlation curves. GFP concentration increases over several hours as cell-free protein synthesis progresses. The diffusion times of the 1-species fits decrease over time (bottom). When GFP is starting to be produced, most of it is bound to the Ab, giving a larger diffusion time. As the reaction produces more GFP to be eventually greater than the amount of Ab, most of the GFP is not bound, thus having a lower diffusion time.

FIG. 12D depicts a concentration curve from GFP, according to this disclosure.

FIG. 12E depicts a correlation to time delay, according to this disclosure.

FIG. 12F depicts diffusion times from GFP, according to this disclosure. Here, a fraction of GFP bound to Ab is obtained from fitting the correlation curves to a 2-species model describing free and bound GFP. The plot of fraction bound vs. concentration is the binding curve, from which the binding affinity is obtained. This method produces a complete binding curve by FCS measurements on one sample solution within a few hours of sample preparation.

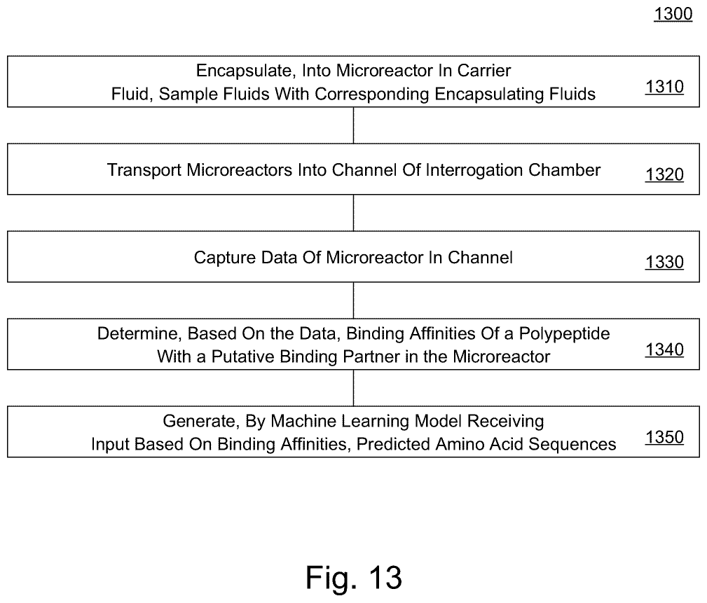

FIG. 13 depicts an example method of automated high-throughput screening, according to this disclosure. At least one of the system of FIG. 1, the apparatus of FIG. 2, the controller architecture of FIG. 5, the interrogation chambers of FIGS. 7-8, the screening instrumentation of FIGS. 10A-B, or any combination thereof, can perform method 1300. At 1310, the method 1300 can encapsulate, into microreactor in a carrier fluid, sample fluids with corresponding encapsulating fluids. At 1320, the method 1300 can transport the microreactors into a channel of an interrogation chamber. At 1330, the method 1300 can capture data of the microreactors in the channel. At 1340, the method 1300 can determine, based on the data, binding affinities of a polypeptide with a putative binding partner in the microreactor. At 1350, the method 1300 can generate, by a machine learning model receiving input based on the binding affinities, predicted amino acid sequences that may have, for example, increased binding affinity to a putative binding partner.

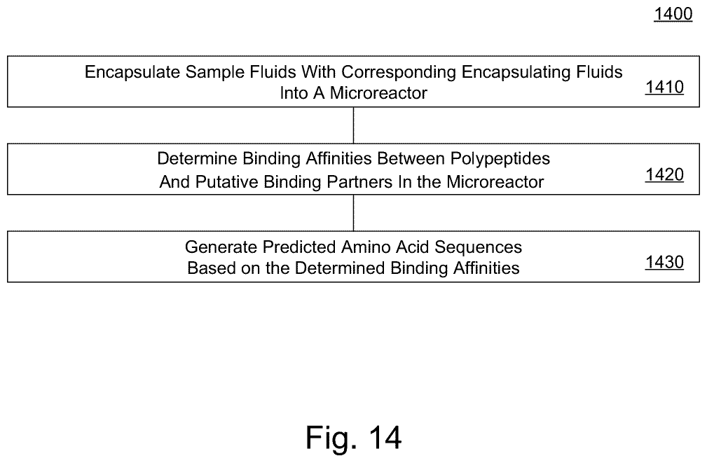

FIG. 14 depicts an example method of automated high-throughput screening, according to this disclosure. At least one of the system of FIG. 1, the apparatus of FIG. 2, the controller architecture of FIG. 5, the interrogation chambers of FIGS. 7-8, the screening instrumentation of FIGS. 10A-B, or any combination thereof, can perform method 1400. At 1410, the method 1400 can encapsulate sample fluids with corresponding encapsulating fluids into a microreactor. At 1420, the method 1400 can determine binding affinities between polypeptides and putative binding partners in the microreactor. At 1430, the method 1400 can generate predicted amino acid sequences based on the determined (or measured) binding affinities that may have, for example increased binding affinity to a putative binding partner.

For example, the method 1300 or 1400 can include generating, based on the one or more predicted amino acid sequences, one or more DNA sequences encoding such an amino acid sequence. For example, the method 1300 or 1400 can include correlating corresponding ones of the one or more microreactors with corresponding ones of the one or more predicted amino acid sequences based on timestamps of the one or more captured data. For example, the method 1300 or 1400 can include depositing, by a robotic device, the one or more sample fluids into corresponding pools, one or more of the pools corresponding to one or more of the predicted amino acid sequences. For example, the method 1300 or 1400 can include depositing performed subsequently to the generating, to iteratively identify amino acid sequences most responsive to (e.g., that can bind to, e.g., with higher binding affinity) a putative binding partner. For example, the method 1300 or 1400 can include holding the microreactors in the channel by an electric field. For example, the method 1300 or 1400 can include generating the electric field by at least one electrode disposed at an exterior surface of the interrogation chamber. For example, the method 1300 or 1400 can include determining a rate of the capturing based on velocity of one or more of the microreactors. For example, the method 1300 or 1400 can include heating the microreactors in the interrogation chamber to increase rate of reaction of the microcapsules. For example, the method 1300 or 1400 can include cooling the microreactors in the interrogation chamber to preserve the microreactors in the interrogation chamber.

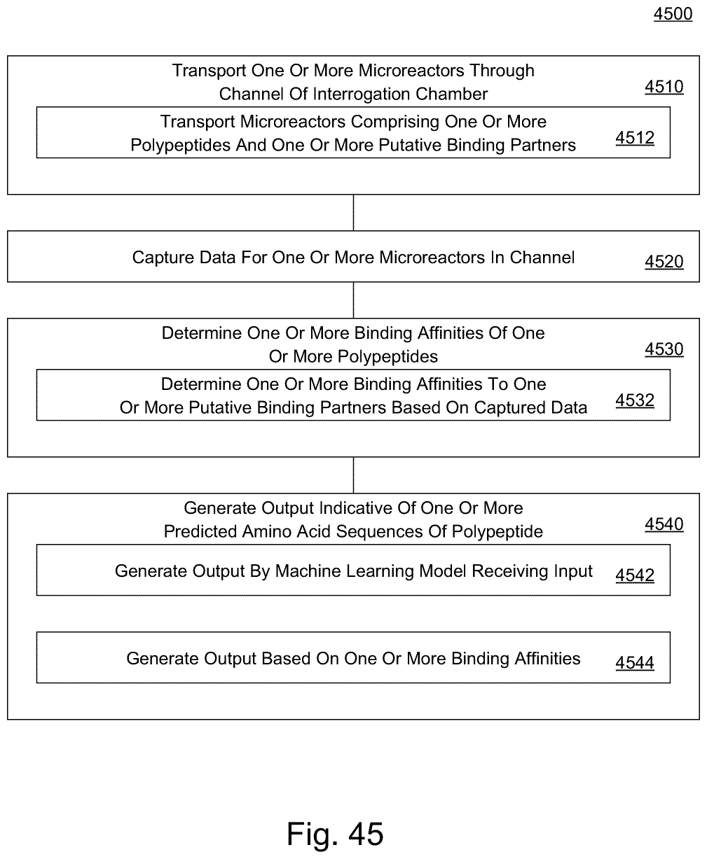

FIG. 45 depicts an example method of automated high-throughput screening, according to this disclosure. At least one of the system of FIG. 1, the apparatus of FIG. 2, the controller architecture of FIG. 5, the interrogation chambers of FIGS. 7-8, the screening instrumentation of FIGS. 10A-B, or any combination thereof, can perform method 4500. At 4510, the method 4500 can transport one or more microreactors through a channel of an interrogation chamber. At 4512, the method 4500 can transport microreactors comprising one or more polypeptides and one or more putative binding partners. At 4520, the method 4500 can capture data for the one or more microreactors in the channel. At 4530, the method 4500 can determine one or more binding affinities of the one or more polypeptides. At 4532, the method 4500 can determine one or more binding affinities to the one or more putative binding partners based on the captured data. At 4540, the method 4500 can generate output indicative of one or more predicted amino acid sequences of a polypeptide. At 4542, the method 4500 can generate output by a machine learning model (e.g., a neural network, a random forest, a support vector machine, a transformer model, a large language model, etc.) receiving input. At 4544, the method 4500 can generate output based on the one or more binding affinities.

For example, the method can include generating, based on the one or more predicted amino acid sequences of the polypeptide, one or more DNA sequences that encode the one or more predicted amino acid sequences of the polypeptide. For example, the method can include correlating corresponding ones of the one or more microreactors with corresponding ones of the one or more predicted amino acid sequences of a polypeptide, based on timestamps of the captured data. For example, the method can include depositing, by a robotic device, each of one or more sample fluids can include one or more polypeptides into a respective container, where each respective container corresponds to one or more of the predicted amino acid sequences of a polypeptide. For example, the depositing is performed subsequently to the generating, to iteratively identify predicted amino acid sequences that can bind, or can bind with increased binding affinity, to the putative binding partner. For example, the one or more putative binding partners comprises an antigen. For example, the one or more putative binding partners comprises a protein of a virus. For example, the one or more polypeptides are comprised in a sample fluid, and where the sample fluid is water-soluble.

For example, the one or more polypeptides comprised in a sample fluid are contacted with a non-water soluble encapsulating fluid to thereby form the one or more microreactors. For example, the one or more microreactors are contacted with a carrier fluid. For example, the method can include where the carrier fluid is glycerine-based. For example, the one or more polypeptides are synthesized using cell-free protein synthesis. For example, the interrogation chamber has a dimension based on a size of the microreactors.

For example, the method can include holding the microreactors in the channel by at least one of an electric field, a magnetic field, pneumatic pressure, fluidic pressure, a permanent gate, or an actuated gate. For example, microreactors are loaded into the chamber until they reach a gate that prevents them from exiting the chamber (while allowing fluid to exit). A gate can be integrated into the interrogation chamber and be actuated to rapidly open or close to ensure control of microcapsule release and retention with high temporal resolution. For example, this gate may be realized by using positive or negative dielectrophoretic (DEP) or electrophoretic forces, optical forces, pneumatic or fluidic pressure to collapse a flexible ceiling, magnetic forces, by electromechanical means such as a physical gate that spans the width of the chamber, etc. The chamber continues to fill with microreactors until it is completely full, or until a user or program stops filling the chamber. A permanent gate (e.g., mechanical filter) or permanently actuated gate may be used in place of a dynamic gate to keep the microcapsules from exiting the chamber. In this embodiment, flow in the chamber can be reversed to wash the microreactors back out the chamber. Once chamber loading is complete, flow into the chamber can be stopped or slowed to ensure microreactors do not drift back out the chamber entrance. For example, the method can include generating the electric field by at least two electrodes disposed at a surface of the interrogation chamber. For example, the surface can be an inner surface of the interrogation chamber within a volume that can be occupied by microreactors. For example, the surface can be an exterior surface of the interrogation chamber in contact with or contactable with an ambient environment.

For example, the method can include tracking, according to the machine learning model, movement of a microreactor among the microreactors to determine a velocity of the microreactor, the velocity indicative of a binding affinity of the microreactor. For example, the method can include heating the microreactors in the interrogation chamber to increase rate of reaction of the microreactors. For example, the method can include cooling the microreactors in the interrogation chamber to preserve the microreactors in the interrogation chamber.

For example, the method can include capturing, by a camera oriented toward the interrogation chamber, first data corresponding to a first microreactor among the microreactors. For example, the system can include determining, based on the captured first data, a first binding affinity of a first instance of the polypeptide in a first instance of the sample fluid comprised within the first microreactor. For example, the camera captures second data corresponding to a second microreactor among the microreactors. For example, the system can include determining, based on the captured second data, a second binding affinity of a second instance of the polypeptide in a second instance of the sample fluid comprised within the second microreactor. For example, the system can include a robotic device to deposit each of one or more sample fluids can include one or more polypeptides into a respective container, where each respective container corresponds to one or more of the predicted amino acid sequences.

For example, the system can include a microfluidic channel to combine one or more sample fluids can include one or more polypeptides with one or more corresponding encapsulating fluids to form one or more microreactors in a carrier fluid. For example, the camera captures first data corresponding to a first microreactor among the microreactors. For example, the system can determine, based on the captured first data, a first binding affinity of a first instance of the polypeptide in a first instance of the sample fluid comprised within the first microreactor. For example, the camera captures second data corresponding to a second microreactor among the microreactors. For example, the system can determine, based on the captured second data, a second binding affinity of a second instance of the polypeptide in a second instance of the sample fluid comprised within the second microreactor.

For example, the sample fluids are water-soluble. For example, the encapsulating fluids are not water-soluble. For example, the microreactors are formed into the carrier fluid. For example, the carrier fluid is glycerine-based. For example, the interrogation chamber has a dimension based on a size of the microreactors.

For example, a system can include a robotic device to deposit one or more sample fluids into corresponding pools, one or more of the pools corresponding to one or more of the predicted amino acid sequences. For example, the system can include a microfluidic channel to combine one or more sample fluids with one or more corresponding encapsulating fluids to form one or more microreactors in a carrier fluid.

Having now described some illustrative implementations, the foregoing is illustrative and not limiting, having been presented by way of example. In particular, although many of the examples presented herein involve specific combinations of method acts or system elements, those acts and those elements may be combined in other was to accomplish the same objectives. Acts, elements and features discussed in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B” can include only ‘A’, only ‘B’, as well as both “A’ and ‘B’. Such references used in conjunction with “comprising” or other open terminology can include additional items. References to “is” or “are” may be construed as nonlimiting to the implementation or action referenced in connection with that term. The terms “is” or “are” or any tense or derivative thereof, are interchangeable and synonymous with “can be” as used herein, unless stated otherwise herein.

Directional indicators depicted herein are example directions to facilitate understanding of the examples discussed herein, and are not limited to the directional indicators depicted herein. Any directional indicator depicted herein can be modified to the reverse direction, or can be modified to include both the depicted direction and a direction reverse to the depicted direction, unless stated otherwise herein. While operations are depicted in the drawings in a particular order, such operations are not required to be performed in the particular order shown or in sequential order, and all illustrated operations are not required to be performed. Actions described herein can be performed in a different order. Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any clam elements.

Scope of the systems and methods described herein is thus indicated by the appended claims, rather than the foregoing description. The scope of the claims includes equivalents to the meaning and scope of the appended claims.

### EXAMPLES

These examples are provided for illustrative purposes only and do not to limit the scope of the claims provided herein.

### Example 1: Materials and Methods

Staging for FCS in microcapsules (double emulsion) and/or bulk fluid assessment of polypeptides produced by cell-free protein synthesis: A custom Python program was used to control the movement of a ThorLabs, Inc. MLS203-1 XY stage so that the FCS system could scan multiple samples at various timepoints, making it possible to generate polypeptide-putative binding partner binding kinetics for each sample. The 60× water immersion objective was attached to a Newport 9063-X-P linear actuation aligned in the vertical orientation to allow fine focusing control on the samples.

Microdroplets (single emulsions): Two Dolomite Mitos P-Pump pressure pumps and a Dolomite junction chip were used to create ˜300 μm diameter aqueous droplets suspended in oil (“microdroplets”). This was accomplished by infusing the aqueous solution (dispersed phase) through the center channel and infusing oil in through the two sheath channels. Both pressure pumps infused oil, but one passes through a VICI Valco 6-port, 2-position injection valve. When the 5 μL coil in the valve was filled with aqueous sample, it was electrically actuated to allow the aqueous slug to be pushed out of the coil by the pressure pump. This sample was then partitioned into microdroplets when it reached the junction chip. Oil and aqueous flow rates each were typically 5-20 μL/min. The oil phase was typically Novec 7500 from 3M Corporation with 5% Pico-Surf surfactant as a stabilizer from Sphere Fluidics (Cambridge, UK). Aqueous phases that have been evaluated and were successfully utilized include: water, food coloring, PBS or water with fluorophores such as GFP, and custom cell-free synthesis lysate with varying amounts of Span 80 surfactant (Sigma-Aldrich) up to 8% (v/v) concentration. Custom lysate solutions in some cases, contained DNA plasmids that encode fluorophores such as mCherry and GFP within the microdroplets.

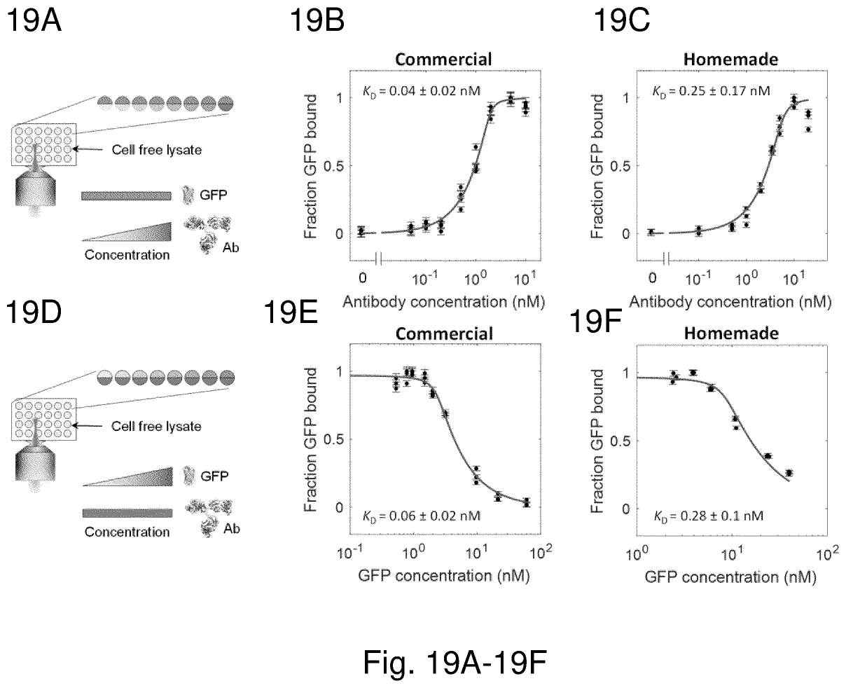

To automate aqueous sample partitioning into microdroplets, a custom Python program directed an OpenTrons OT-2 fluid handler to inject aqueous samples into the injection valve, filling the coil was utilized. The program then electronically switched the injection valve to partition the sample in the downstream junction chip. This, was in some cases, done repeatedly for various samples as long as enough time had passed for the 5 μL sample to be fully purged out of the coil before the next sample was injected. After the sample was partitioned into microdroplets, the microdroplet stream was directed into the parking lot (described in FIGS. 19A-19F, FIG. 24, FIG. 40). Multiple samples were directed sequentially to the parking lot created sample bands within the parking lot, which were separated by partitions of droplets containing only food coloring. This was used to help distinguish different droplet groups from another. The parking lot was filled with droplets, then either flow was stopped or the droplet stream was diverted to waste upstream of the parking lot entrance.

Preparation of Cell-free Protein Synthesis (CFPS) system: Overnight starter cultures of ClearColi BL21 (DE3) (Biosearch Technologies) were grown in 2× YT media supplemented to 1% NaCl at 37° C., 225 rpm were diluted 1:50 into multiple 2L baffled shake flasks containing 500 mL of 2× YT/1% NaCl media. Cultures were grown until OD600 reached 0.5-0.7 and induced with 1 mM isopropyl β-d-1-thiogalactopyranoside (IPTG) for 4 hours. Cells were harvested by centrifugation at 8000 rpm in an SLA-3000 rotor, washed with Buffer A (10 mM Tris base, 14 mM magnesium glutamate, 60 mM potassium glutamate, 1 mM DTT), centrifuged again at 6000 rpm, and resuspended with 1 mL of Buffer A per gram of wet cell mass and frozen at −80° C. Roughly 5 g of wet mass per liter of media was generally obtained.

After thawing, cells were lysed through sonication using a qSonica Q500 probe-tip sonicator with ⅛″-¼″ probes, dependent on sample volume, in 30 s on: 30 s off cycles and 25% amplitude until 3,000kJ of input energy was reached. Cell suspension turned a darker shade of brown as lysis increased. An additional 1 mM dithiothreitol (DTT) was added after sonication. Sonicated lysates were transferred into 1.5 mL microcentrifuge tubes and centrifuged at 18,000 rcf for 10 min at 4° C. and the supernatant transferred to fresh 1.5 mL microcentrifuge tubes. Lysates were incubated at 37° C. for 30 minutes to perform the run-off reaction and centrifuged again at 10,000 rcf for 10 minutes at 4° C. Supernatant was transferred to fresh 1.5 mL microcentrifuge tubes before flash freezing and storage at −80° C.

Cell-free protein synthesis reaction: CFPS reactions (1 mL scale) were prepared in 1.5 mL microcentrifuge tubes by combining lysate (25% final reaction volume), plasmid, and water with the following components: 1.2 mM ATP; 0.86 mM guanosine triphosphate (GTP), uridine triphosphate (UTP), and cytidine triphosphate (CTP); 34 μg/mL folinic acid; 170 μg/mL E. coli transfer RNA (tRNA); 2 mM of each amino acid (-glutamic acid), 0.33 mM nicotinamide adenine dinucleotide (NAD); 0.27 mM Acetyl-CoA; 1.5 mM spermidine; 1 mM putrescine; 175 mM potassium glutamate; 10 mM ammonium glutamate; 2.7 mM potassium oxalate; 10 mM magnesium glutamate; and 33 mM phosphoenolpyruvate (PEP). 0-10 μM pET-15b plasmid encoding His-Avi-tagged GFP DNA was used for each reaction. 6.7μM of anti-GFP antibody was included as appropriate unless otherwise indicated. CFPS reactions were loaded into glass syringes and kept on ice before connecting to syringe pumps and microfluidic device for encapsulation.

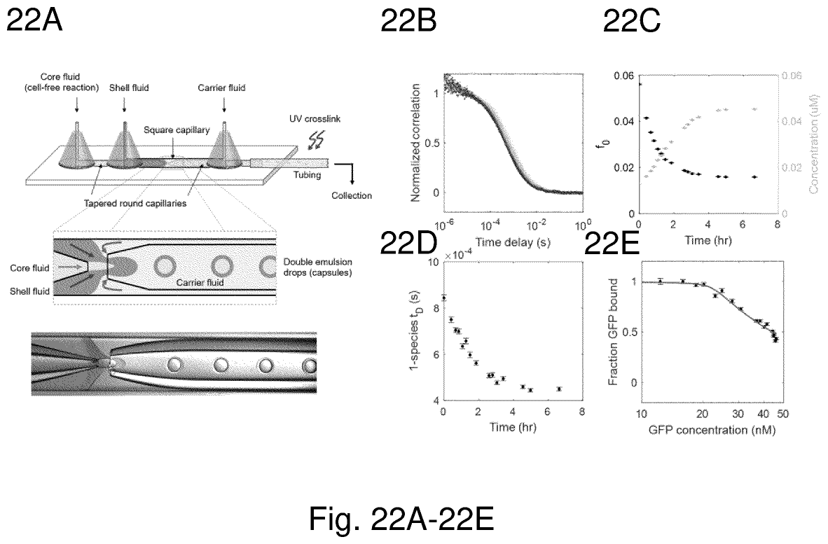

Device Fabrication: The microcapillary device was fabricated as discussed. Briefly, the base of the device was formed by bridging two 2 by 3 inch glass slides by epoxy and two small glass strips. A round glass capillary (15.24 cm long with an outer diameter of 1.0 mm and inner diameter of 0.580 mm, World Precision Instruments, Sarasota, FL) and a square capillary (with an internal width of 1.0 mm, VitroCom, Mountain Lakes, NJ) composed the main components of the device. The square capillary was glued to the base after being cut to the desired length. The round capillary was centered in a pipette puller (Model P-97, Sutter Instruments, Novato, CA) to decrease its diameter in the center under tension and heat, breaking into two equally tapered capillaries. The tapered glass capillaries were then cleaved to the desired final diameters using a microforge station (Micro Forge MF 830, Narishige, Japan). Typical inner diameters of the capillaries ranged from 20 μm to 300 μm. After cleaning in an ethanol solution with 10 minutes of sonication, tips were treated separately with different silane solutions to change the glass hydrophilicity and hydrophobicity. The inner fluid capillary tip was treated to be hydrophobic so that the aqueous inner fluid could be easily repelled to break up into drops at the end of the capillary. Similarly, hydrophilic coating was applied to the exit capillary to accelerate the breakup of the oil-based middle fluid. Schematics of the device is shown in FIG. 22A.

Microencapsulation: Syringe pumps (Harvard PHD Ultra, Harvard Apparatus, Holliston, MA) were used to pump inner (core), middle (shell), and outer (continuous phase) into the devices. Once the device was filled with liquid, flow rates were adjusted for obtaining stable double emulsion drop formation. After microdroplets exited the device, they continued to travel to a UV-crosslinking section where a 365 nm UV lamp (UVP Multiple-Ray Lamp, Fisher Scientific, Hampton, NH) crosslinked the polymer in the shell phase, producing microcapsules. Glass vials were typically used for capsule collection by inserting the end of the exit tubing into the vial prefilled with 10 ml of PBS solution. Emulsion drops were typically collected for a period of 15-20 minutes. Then the continuous fluid was replaced by using filtration tool to remove the original continuous fluid and resuspend the capsules in fresh solution containing 10wt % glycerol and 2 wt % poly vinyl alcohol for osmotic balancing with the core fluid. Drop production was visualized on a microscope equipped with a fast camera (Photron Mini AX100, Photron, San Diego, CA) capable of up to 540,000 frames/sec. Images of the double emulsion drops and capsules were analyzed with the freely available image analysis program, ImageJ (Rasband, W.S., U. S. National Institutes of Health, Bethesda, MD).

### Example 2: FCS Tracks Fluorescent Polypeptides Synthesized by CFPS

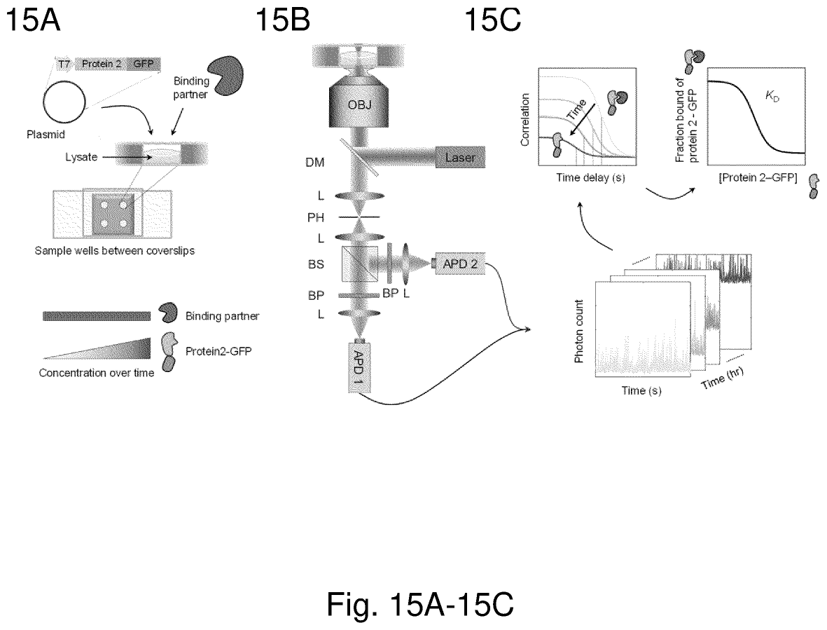

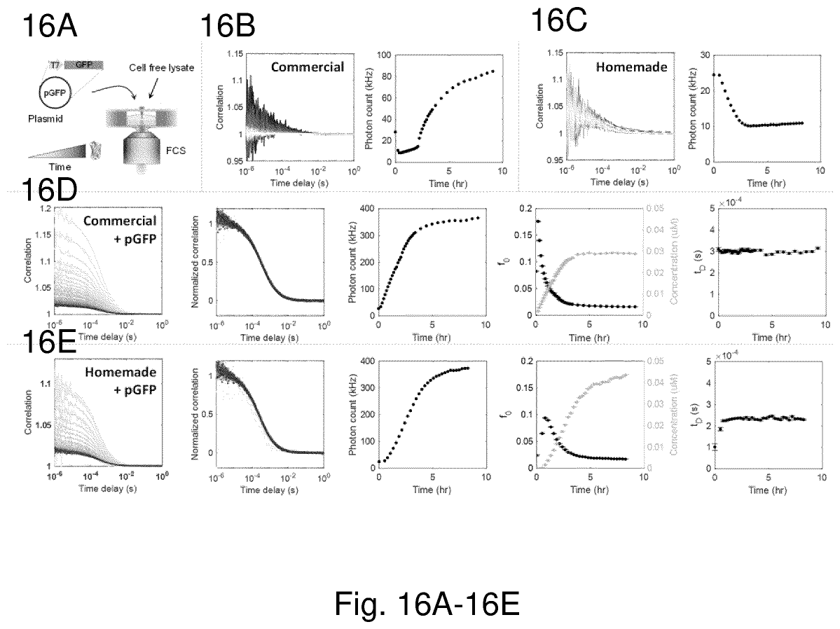

FIGS. 15A-15C shows an experimental set up to measure binding of a polypeptide during cell-free synthesis to a putative binding partner by fluorescence correlation spectroscopy (FCS). In the present example, Green fluorescent protein (GFP) was produced by cell-free protein synthesis and measured by FCS over time (FIG. 16A). First, a commercially available lysate (Rabbit Biotech) and a “homemade” lysate were prepared without plasmid, CFPS was conducted, and measurements were taken using FCS. Results shown in FIGS. 16B and 16C demonstrate correlation noise from the lysate background (left) and photon counts (right). Photon counts for both lysates initially decreased before increasing, and the “homemade” lysate had lower lysate background signal. The commercially available and “homemade” lysates were alternatively prepared with 1 μg/mL plasmid encoding GFP, CFPS was conducted, and measurements were taken using FCS. Results shown in FIGS. 16D and 16E show correlation curves, photon count, fitted amplitudes and diffusion times over experimental time. The signal due to GFP is much greater than that due to lysate background, giving clean correlation curves. GFP production saturates within around 5 hours under these experimental conditions of 1 μg/ml plasmid and room temperature. Such results demonstrate that FCS can measure the production of a fluorescent polypeptide in the unpurified environment of cell-free lysates and can accurately determine the concentration and diffusion time at each time point.

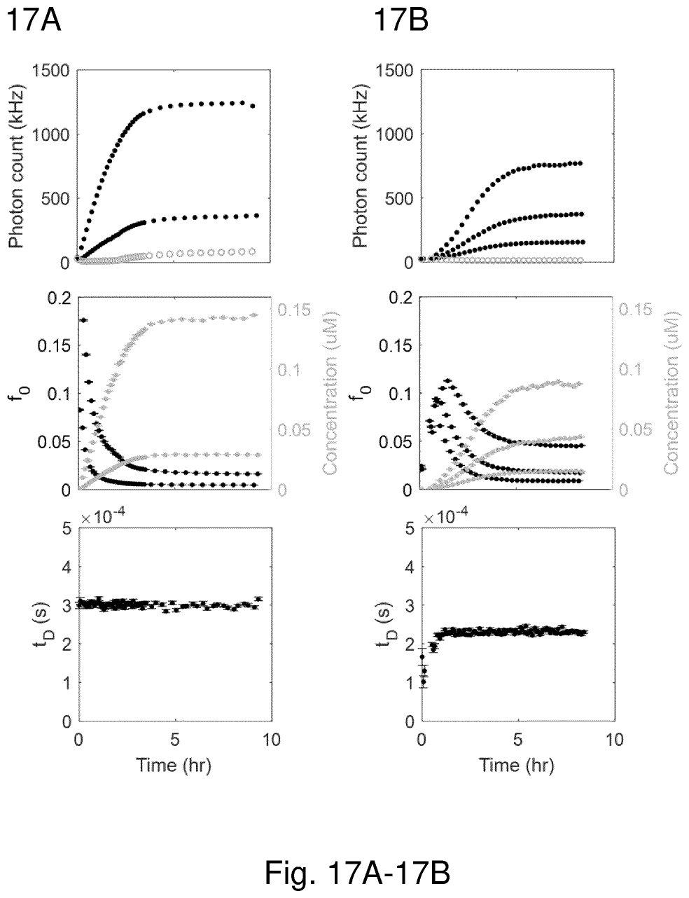

Next, it was determined whether the rate and amount of polypeptide synthesis can be tuned by altering the plasmid concentration in the CFPS reaction. Varying amounts (e.g., 5 μg/ml and 1 μg/ml for FIG. 17A, 2 μg/ml, 1 μg/ml, and 0.5 μg/ml for FIG. 17B) of plasmid comprising a nucleotide sequencing encoding GFP were utilized. Photon count rate, flitted amplitude, and diffusion times were evaluated over time for the varying GFP plasmid concentrations in either commercial (FIG. 17A) or “homemade” (FIG. 17B) cell-free lysates. Results in FIGS. 17A and 17B demonstrate that the “homemade” lysate had slightly slower production rates than the commercial lysate, but similar final yields of polypeptide.

### Example 3: Evaluation of Binding Affinity Between GFP and Anti-GFP Antibody

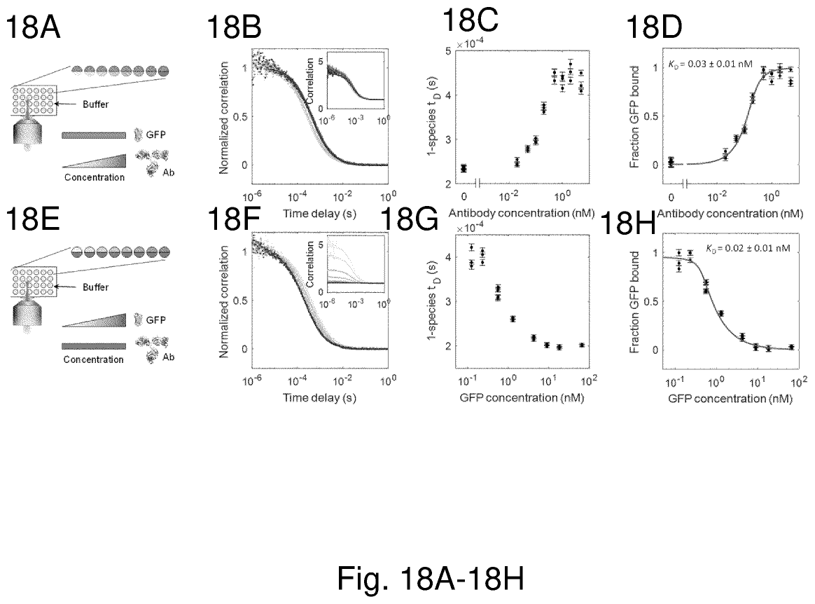

Binding affinity between GFP and anti-GFP antibody was evaluated. First, to measure the binding affinity between GFP and an anti-GFP antibody, samples containing a constant concentration of GFP (200 pM) and varying concentrations of the unlabeled anti-GFP antibody in buffer were prepared and measured by FCS (FIG. 18A). FIG. 18B shows that normalized concentration curves shifted right at higher antibody concentrations, while FIG. 18C shows that diffusion times from one-species fits to correlations increased as a function of antibody concentration. The fraction of GFP bound to antibody was calculated and a K_{D} of 0.03+−0.01 nM and antibody concentration P=0.19+−0.03 nM were determined, which aligned with the known amount of GFP utilized (FIG. 18D). A binding curve was also obtained by varying the GFP concentration while utilizing a constant concentration of the unlabeled anti-GFP antibody (400 pm) (FIG. 18E). Normalized correlation curves shifted left at higher GFP concentrations (FIG. 18F) and diffusion times from one-species fits to correlations decreased as a function of GFP concentration (FIG. 18G). The fraction of GFP bound to antibody was calculated and a K_{D} of 0.02+−0.01 nM and antibody concentration P=0.41+−0.02 nM were determined which was consistent with both values determined by the buffer-based binding assessment method and the known amount of antibody utilized (FIG. 18H).

Next, the binding affinity between GFP and an anti-GFP antibody in cell-free lysates was evaluated. Control experiments as described in FIGS. 18A-18H, varying either unlabeled antibody (FIGS. 19A-19C) or GFP (FIGS. 19D-19F), were repeated in commercial or “homemade” cell-free lysates. Higher amounts of the non-varying polypeptide (e.g., GFP, anti-GFP antibody) were required than in the corresponding buffer experiments described in FIGS. 18A-18H to measure GFP signals above the higher background because lysates have higher background signal than buffer.

FIG. 19B shows results of the fraction of GFP bound to the anti-GFP antibody using 2 nM GFP and varying concentrations of anti-GFP antibody in commercial lysate. A K_{D} of 0.04+−0.02 nM and GFP concentration P=1.9+−0.1 nM were determined. FIG. 19C shows results of the fraction of GFP bound to the anti-GFP antibody using 5 nM GFP in a homemade lysate and varying concentrations of anti-GFP antibody. A K_{D} of 0.25+−0.17 nM and GFP concentration P=5.3+−0.6 nM were determined.

FIG. 19E shows results of the fraction of GFP bound to the anti-GFP antibody using 2 nM anti-GFP antibody and varying concentrations of GFP in commercial lysate. A K_{D} of 0.06+−0.02 nM and anti-GFP antibody concentration P=2.2+−0.1 nM were determined. FIG. 19F shows results of the fraction of GFP bound to the anti-GFP antibody using 10 nM of anti-GFP antibody and varying concentrations of GFP in homemade lysate. A K_{D} of 28+−0.1 nM and anti-GFP antibody concentration P=8.1+−0.4 nM were determined.

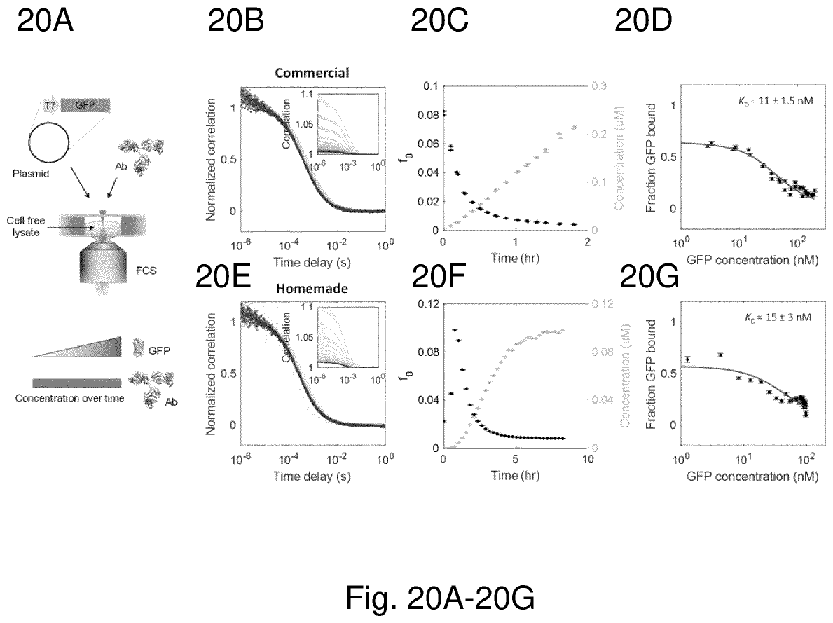

Binding affinities between GFP and anti-GFP antibody as determined by FCS of protein synthesis over time in cell-free lysates were evaluated. Samples containing a plasmid comprising a nucleotide sequence encoding GFP and anti-GFP antibody in either a commercial or “homemade” cell-free lysate were measured over time by FCS for production of GFP and antibody binding (FIG. 20A). Using commercial cell-free lysate, normalized correlation curves (FIG. 20B) shifted left as GFP concentration increased over time (FIG. 20B, inset). Total GFP concentrations were determined from the total amplitude (f₀) of two-species fits and background subtracted using lysate only data at each time point (FIG. 20C). The fraction of GFP bound was calculated from two-species fits and was plotted against the measured GFP concentrations (FIG. 20D). The variant quadratic binding equation was fitted to the measurements to obtain a K_{D}=11+−1.5 nM and antibody concentration P=19+−1.5 nM. Using “homemade” cell-free lysate, normalized correlation curves (FIG. 20E), total amplitudes and background-subtracted concentrations (FIG. 20F), and binding curves (FIG. 20G) for measurements using 0.5 nM plasmid and 20 nM anti-GFP antibody are shown. Interestingly, amplified was initially low (FIG. 20F). Without wishing to be bound by any one theory, it is understood this was due to the dominant contribution of lysate background to the total signal, as also evident in the earliest correlations (FIG. 20E). A fitted KD of 15+−3 nM and antibody concentration P=20+−2 nM were determined. The fitted antibody concentrations determined were consistent with the known amount (20 nM) utilized. Since the concentration of antibody was required to be sufficiently high to measure signal above lysate background, and was much higher than the true low-picomolar K_{D} (FIGS. 18A-18H, FIGS. 19A-19F), these real-time binding curves establish an upper bound on the K_{D}. While the exact K_{D} was not determined, these low-nanomolar upper bounds are nevertheless very informative: the results indicate that the pair has a binding affinity at least as strong as the upper bound. Furthermore, most biologically relevant interactions are in the nanomolar to micromolar range, thus for other interaction pairs that have higher true K_{D}s, this method would be able to precisely determine those K_{D}s.

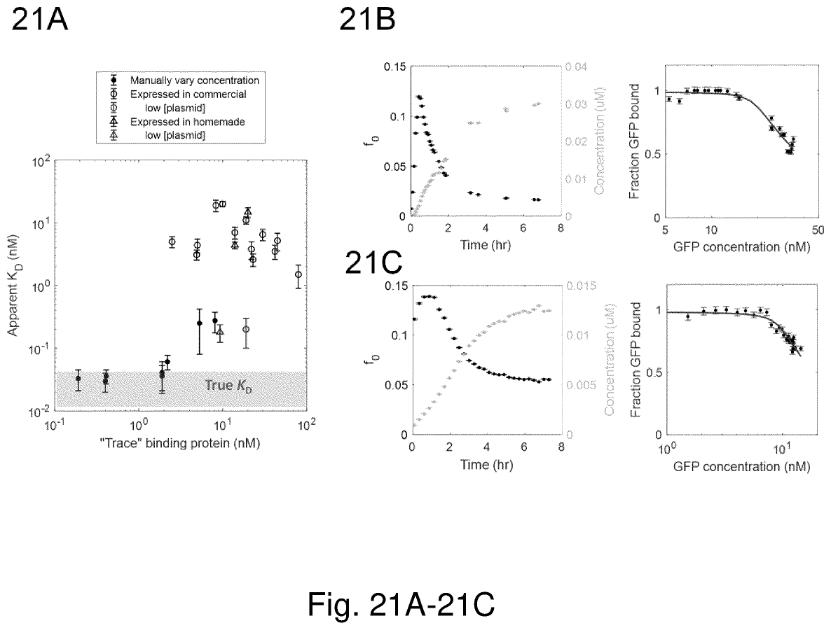

The use of higher concentration of putative binding partner to establish an upper bound on the K_{D} measured by cell-free protein synthesis was further evaluated. A compilation of the measured K_{D} vs. the putative binding partner concentration (P) from conducted experiments was produced (FIG. 21A). Accurate determination of K_{D} typically requires that P<K_{D}. Such a condition is not often met in biological experiments due to practical constraints. Control experiments, in which sub-nanomolar P (antibody or GFP concentration) were used (solid markers from FIGS. 18A-18H, 19A-19F), suggested that the true affinity was ˜30 pM or less (shaded area). Interestingly, much higher K_{D} values were measured from real-time cell-free protein synthesis experiments (open markers) due to a combination of the much higher P (antibody concentration) required in lysate background, GFP maturation time, and/or non-equilibrium binding. Slowing down and lowering protein expression produced K_{D} measurements more consistent with control experiments (red open markers). FIG. 21B shows results from binding experiments from cell-free protein synthesis using 0.12 nM (0.5 μg/mL) plasmid encoding GFP and 20 nM anti-GFP antibody in commercial lysate. Total GFP concentrations were determined from the total amplitude (f₀) of two species fits and background subtracted using lysate-only data at each time point (FIG. 21B, left). The fraction of GFP bound was calculated from two-species fits and was plotted against measured GFP concentrations. The variant quadratic binding equation was filled to the measurements and obtained a K_{D}=0.2+−0.1 nM and antibody concentration P=18.7+−0.5 nM (red open circle in FIG. 21A). Total amplitudes and back-ground subtracted concentrations (FIG. 21C, left) and binding curve (FIG. 21C, right) are shown for measurements of 0.5 μg/ml (0.12 nM) plasmid and 20 nM antibody in homemade lysate. A fitted K_{D}=0.18+−0.06 nM and antibody concentration P=9.3+−0.2 nM (red open triangle in FIG. 21A) were determined.

### Example 4: Determination of Binding Affinity from Cell-Free Protein Synthesis Inside Microcapsules

Binding affinities between polypeptide (GFP) produced by cell-free protein synthesis and putative binding partner (anti-GFP antibody) inside microcapsules was evaluated. FIG. 22A (top) shows a schematic of double emulsion droplet (“microcapsule”) generation on a microfluidic device. In brief, through the flow of fluids in two round capillaries with tapered openings within a larger square capillary, the core fluid (cell-free reaction) (yellow) is packaged into droplets with a polymer shell (red) surrounded by the carrier fluid (light blue). Droplets pass under a UV lamp to crosslink the shell polymer before collection and measurement of the capsules by FCS. FIG. 22A (bottom) shows an image of microcapsule formation in the region of the interface between the three fluids. FIGS. 22B-22E show FCS measurements of protein synthesis and binding inside a microcapsule that contained 1.45 μg/mL (0.34 nM) plasmid encoding GFP and 20 nM anti-GFP antibody in commercial lysate. FIG. 22B shows normalized correlation curves shifted left as GFP concentration increased over time (FIG. 22B, inset). Total GFP concentrations were determined from the total amplitude f₀ of two-species fits and background subtracted using lysate-only data at each time point, results of which are shown in FIG. 22C. FIG. 22D demonstrates that diffusion times from one-species fits decreased over time as GFP concentration increased. The fraction of GFP bound was calculated from two-species fits and was plotted against the measured GFP concentrations. The variant quadratic binding equation was fitted to the measurements to determine K_{D}=0.11+−0.08 nM and antibody concentration P=22.2+−0.5 nM (FIG. 22E).

### Example 5: Assessment of Microreactors in “Parking Lot”

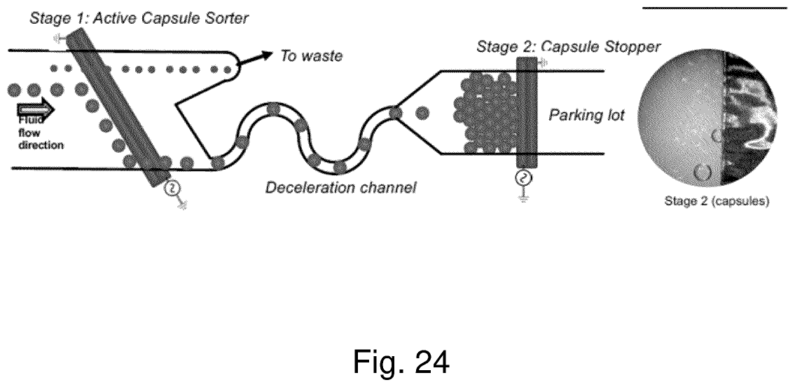

As described herein, an interrogation chamber of the present disclosure can comprise electrodes integrated into the chamber to create a DEP exit gate or “parking lot” that can hold microcapsules within the chamber while they are being loaded or undergoing analysis (FIG. 24).

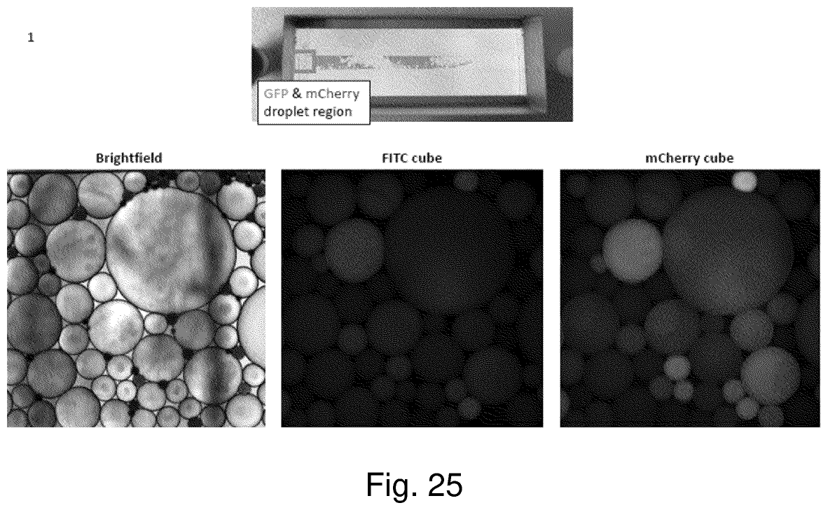

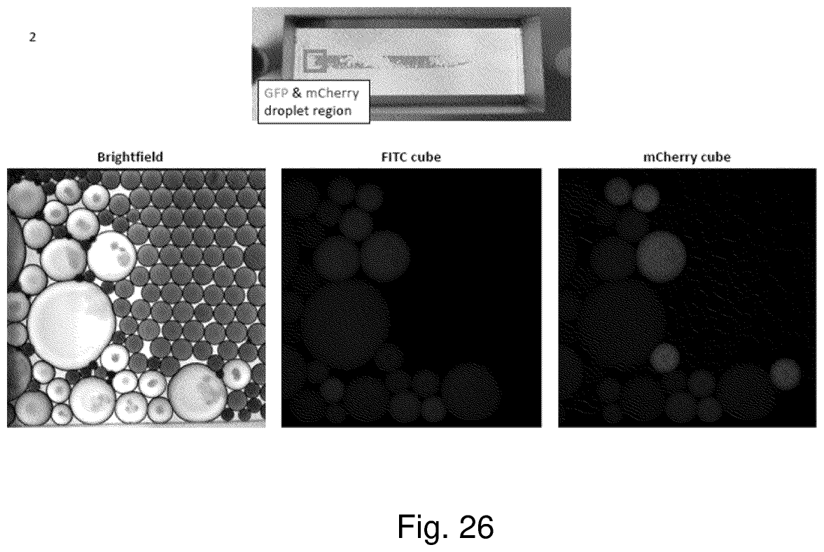

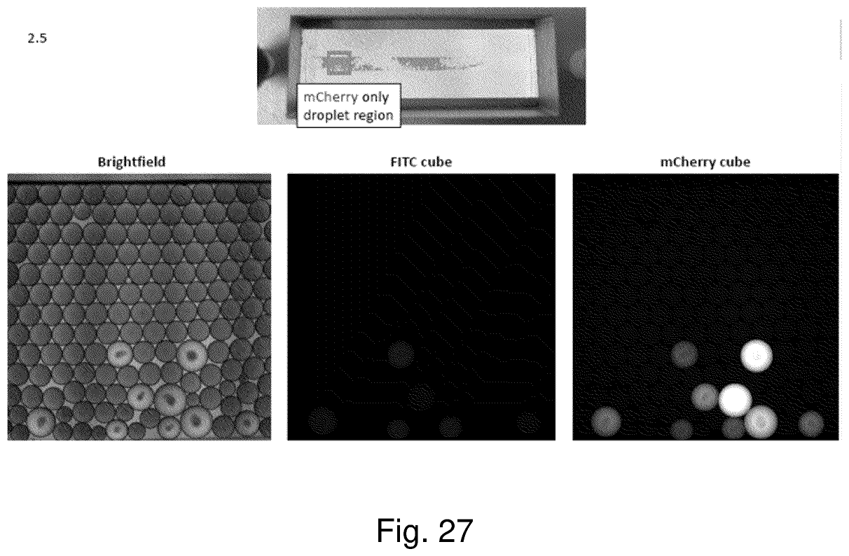

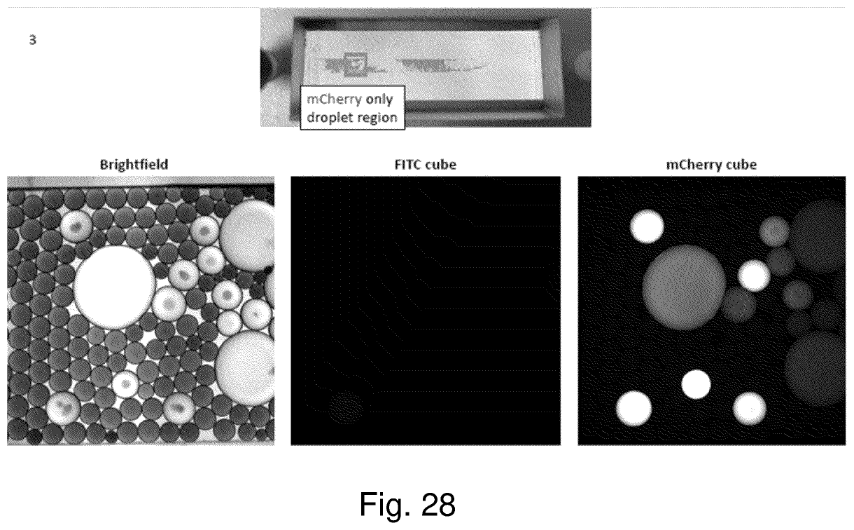

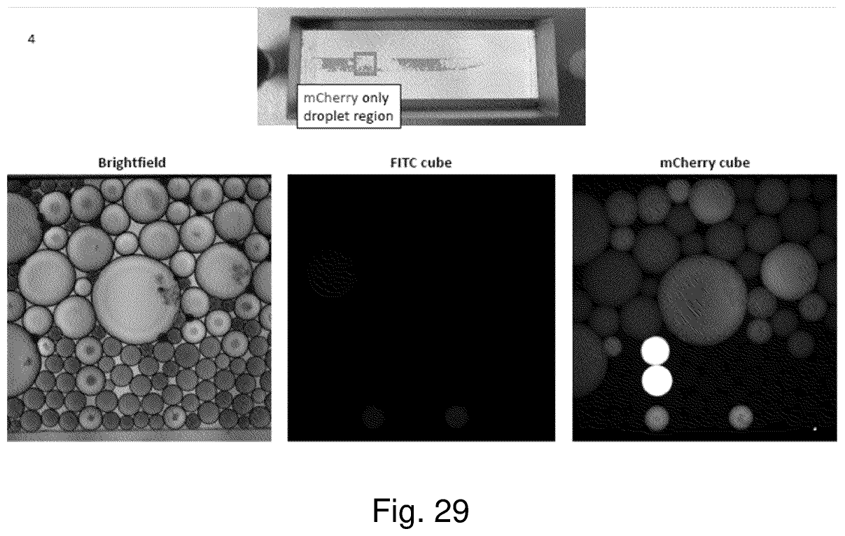

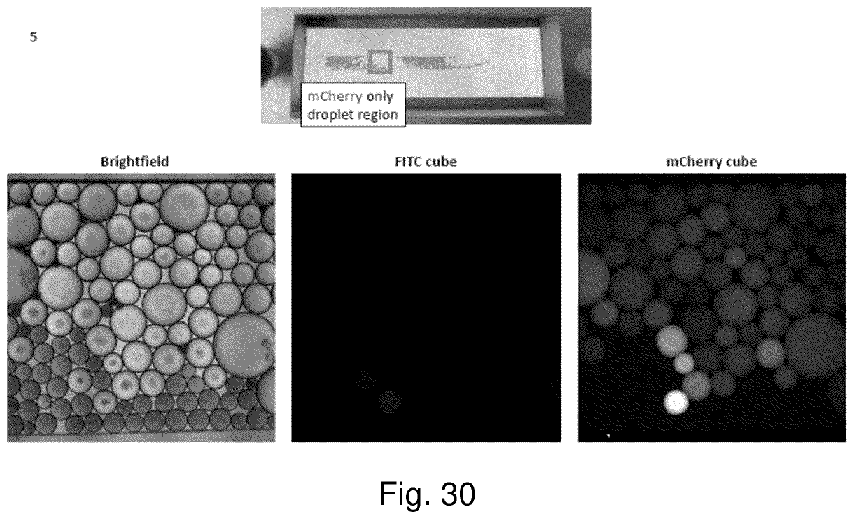

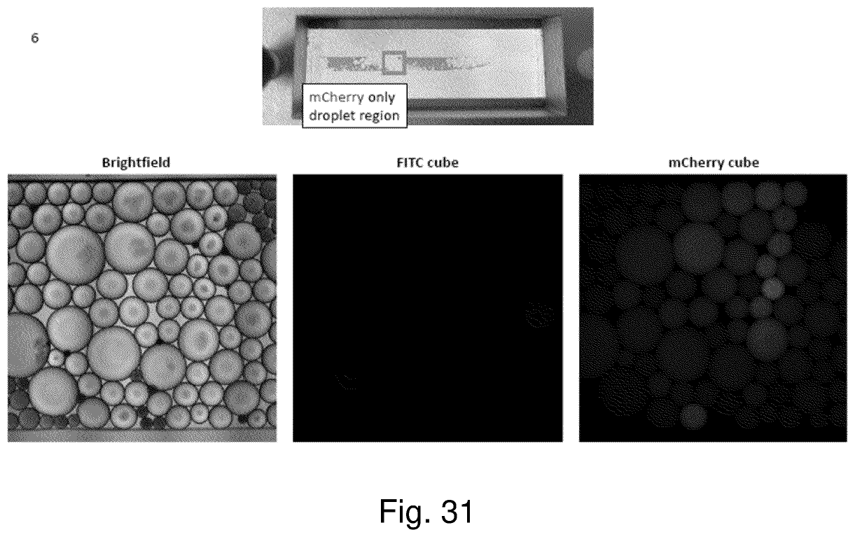

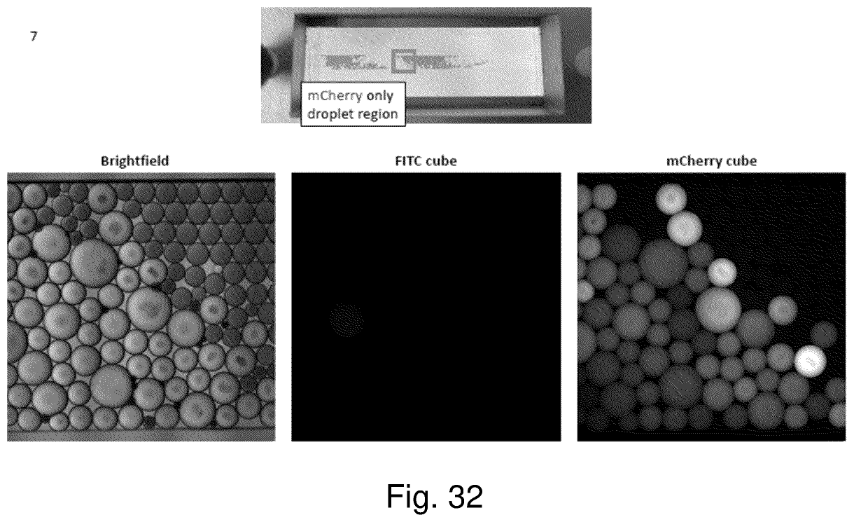

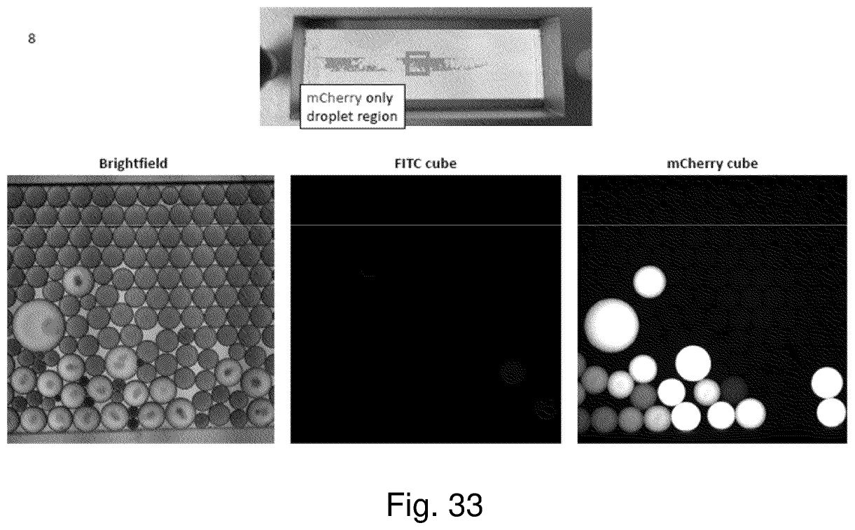

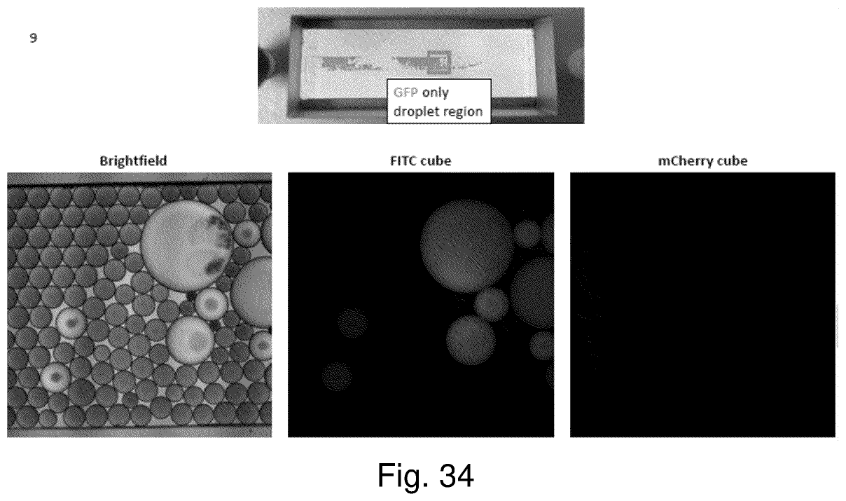

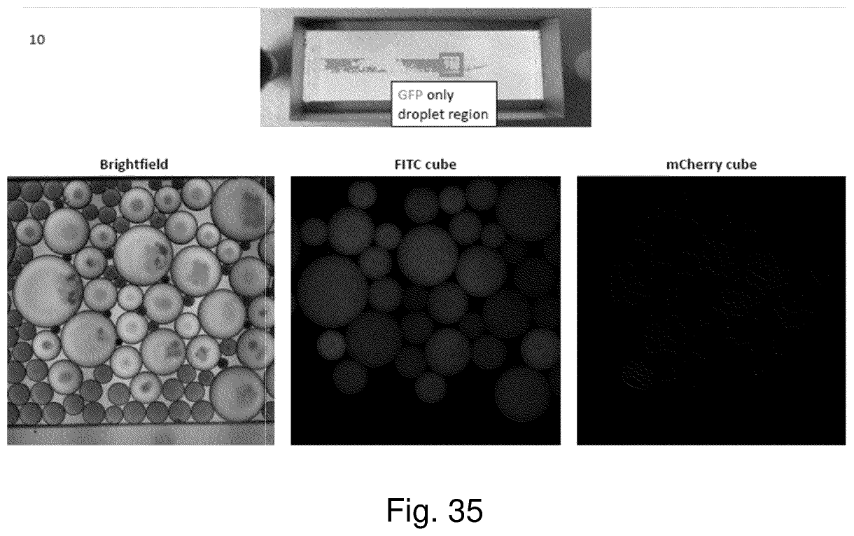

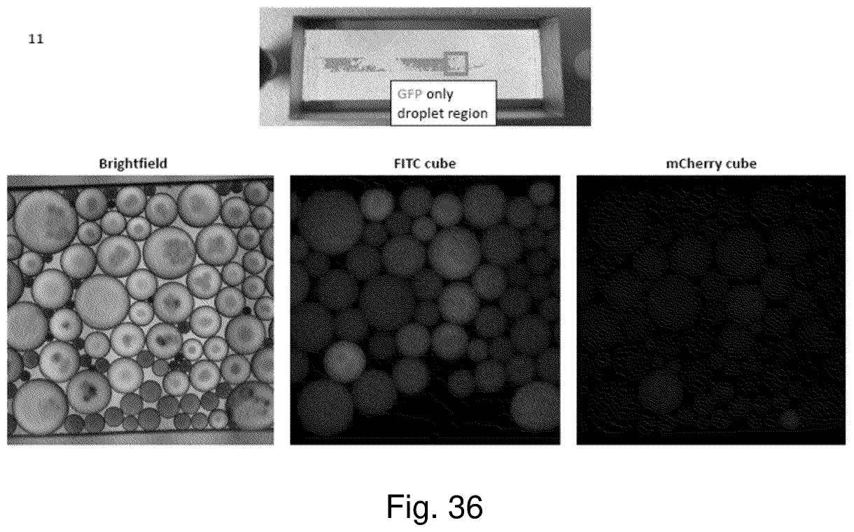

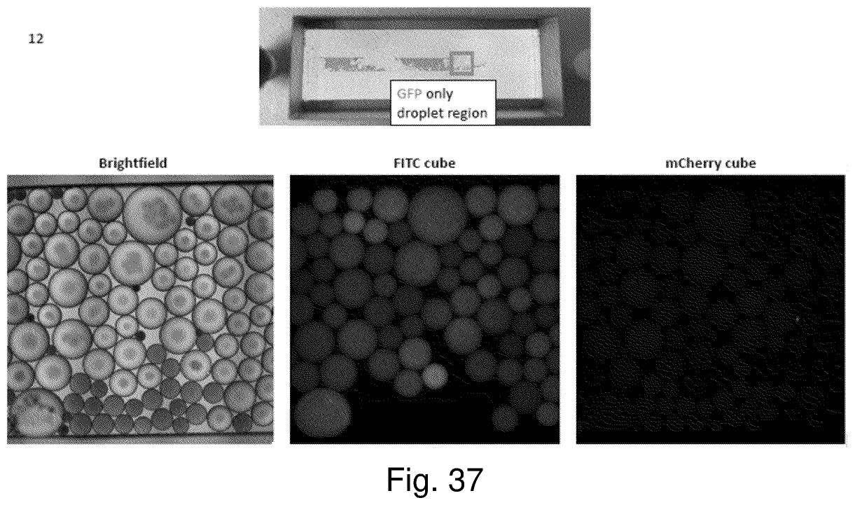

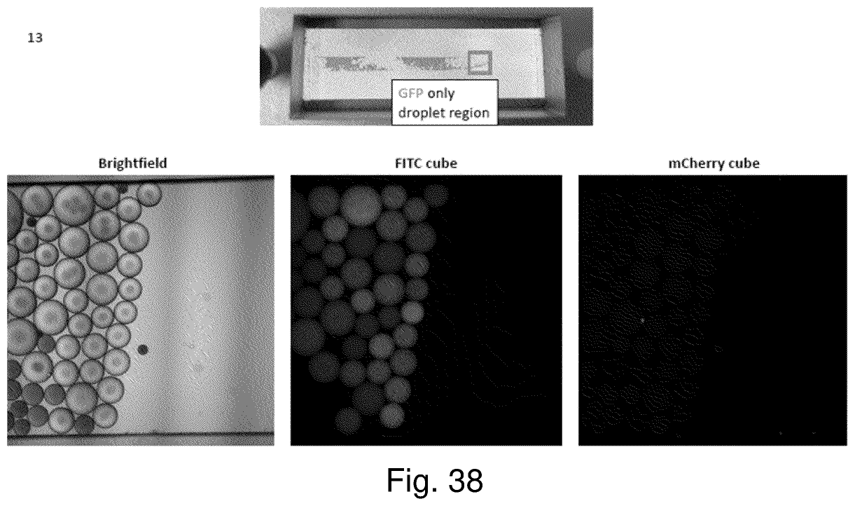

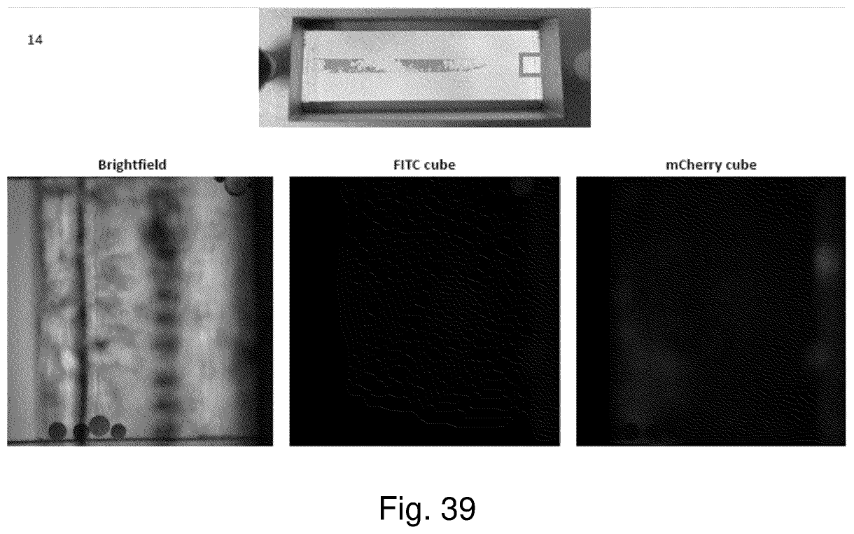

To evaluate microdroplets within the “parking lot” (without a DEP exit gate) using FCS, microdroplets comprising cell-free lysates comprising a DNA template encoding mCherry and GFP were prepared. A silicon gasket of 250 μm thickness was used to create a parking lot with ˜250 μm height. The “parking lot” was filled with several groups of lysate microdroplets with either different concentrations of the same DNA template or with templates for different fluorescent proteins and 2% Span80 as a stabilizer for the microdroplets. Oil used was Novec 7500 with 5% Pico-Surf as a carrier fluid for microdroplet formation and maintenance. Each group of lysate microdroplets were separated by a group of microdroplets filled with blue food coloring. Fluorescent images were taken across several regions (regions shown by orange boxes, FIGS. 25-39) within the parking lot to visualize the different fluorescent proteins that were synthesized within the different microdroplet groups. Images are shown in FIGS. 25-39 and demonstrate that different polypeptides can be synthesized within different microdroplets in the same interrogation chamber at the same time.

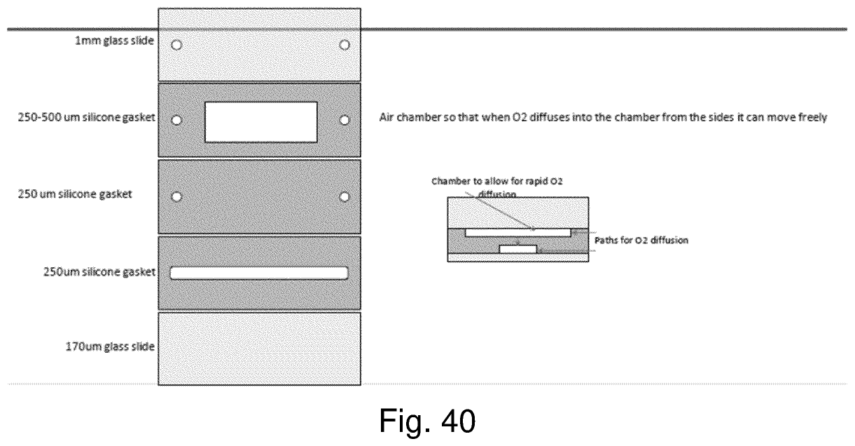

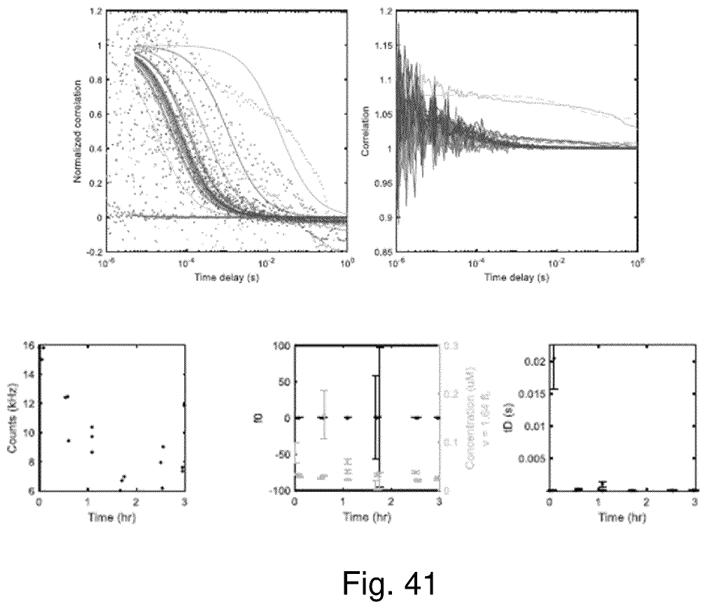

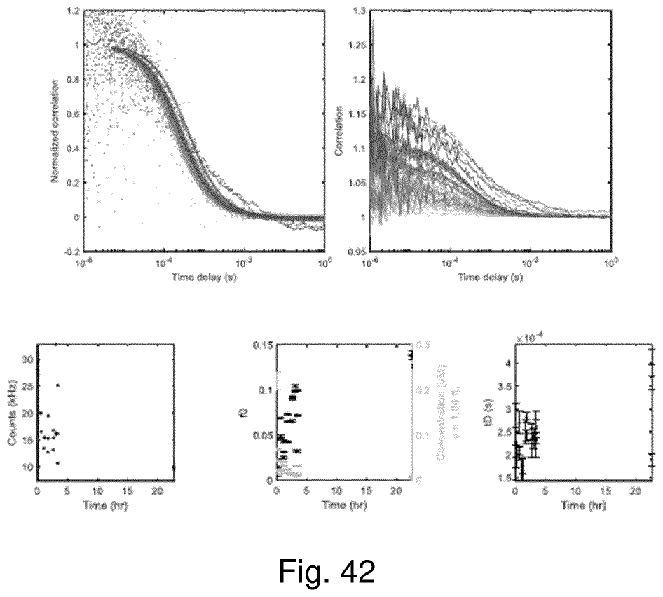

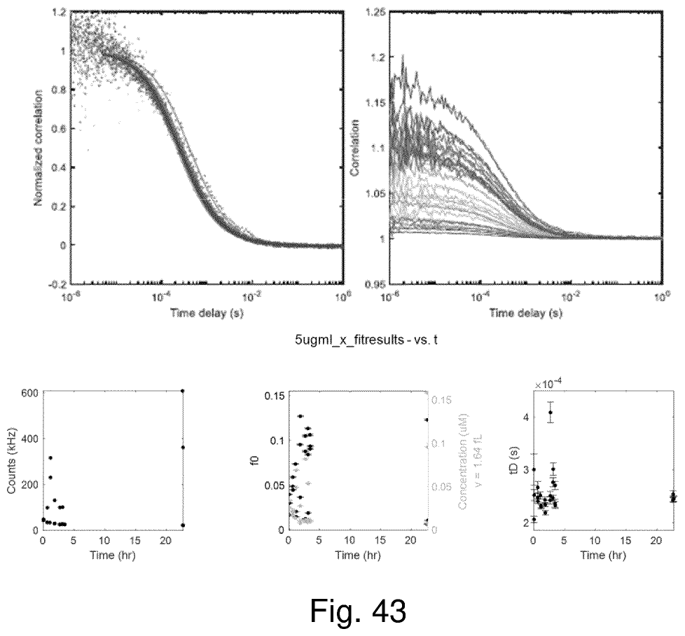

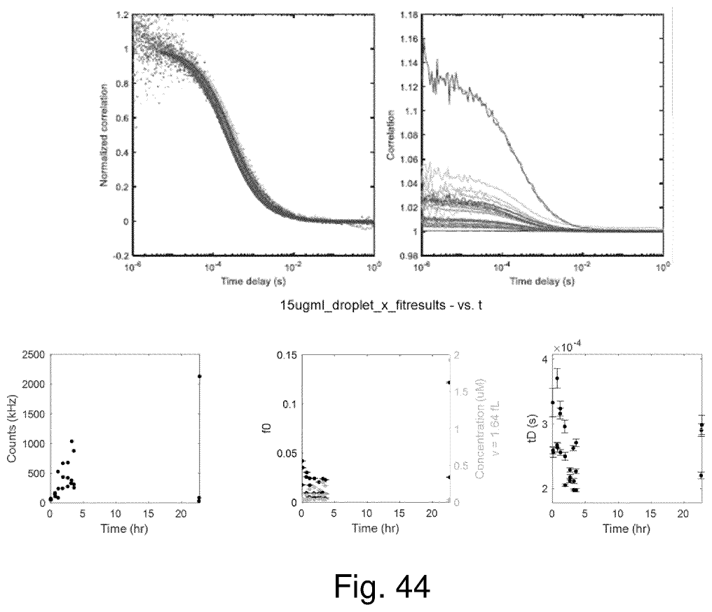

Further evaluation of GFP expression from CFPS in droplets was conducted by loading droplets into “parking lots” with varying concentrations (0, 1, 5, or 15 μg/mL) of plasmid encoding GFP and 2% Span80. A silicon gasket of 250 μm thickness was used to create a parking lot, which was filled with oil (Novec 7500 with 5% Pico-Surf). A schematic of the “parking lot” is shown in FIG. 40. FCS results from the 0 μg/mL plasmid condition, 1 μg/mL plasmid condition, 5 μg/mL plasmid condition, and 15 μg/mL plasmid condition are shown in FIGS. 41-44, respectively. Results demonstrated that GFP production was observed over time.

### Example 6: Assessment of Parallel FCS for CFPS in Microdroplets

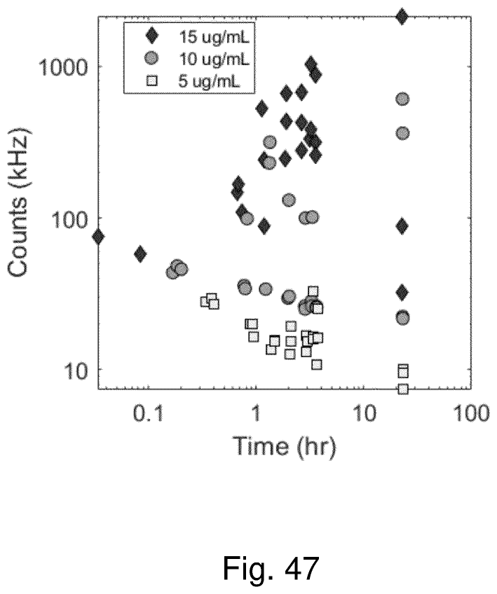

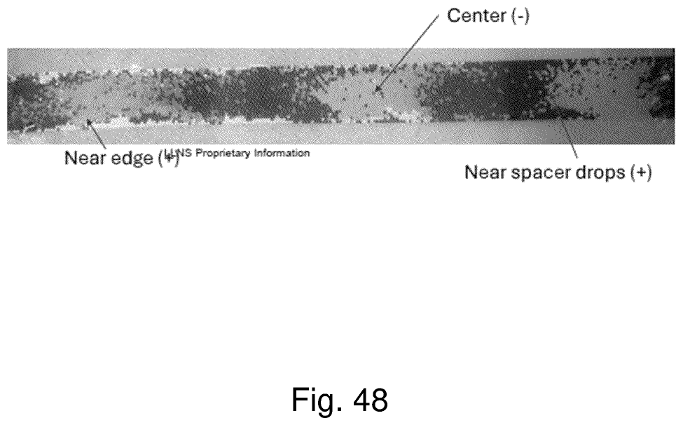

The present example demonstrates, among other things, assessment of multiple microdroplets by FCS at the same time (also referred to as “parallel FCS”). Microdroplets containing either 5 μg/mL, 10 μg/mL, or 15 μg/mL of a plasmid comprising a nucleotide sequence encoding GFP in cell-free lysate were measured over time by FCS for production of GFP. Each group of microdroplets was separated by partitions of microdroplets containing a blue dye as a visual aid (“spacer” microdroplets). In situ FCS measurements of GFP within microdroplets from each group were taken periodically over about a 20-hour time course. The microscope stage was cycled periodically to ensure the same microdroplets were interrogated. Results demonstrated that increased concentrations of GFP plasmid resulted in the highest brightness (FCS counts; FIG. 47). Without wishing to be bound by any one theory, it is understood that the scatter in this data is a result of dependency of GFP activity on microdroplet position, there being higher signal for microdroplets near the edge of the interrogation chamber (e.g., due to access to air/oxygen through the gasket) and near the “spacer” microdroplets that form the partitions between sample microdroplets (e.g., due to spacer microdroplets containing unexpired oxygen) and there being lower signal for microdroplets within the center of their respective group (e.g., due to being away from oxygen sources). See, FIG. 48. Accordingly, these results demonstrate the importance of measuring oxygen levels in systems of the present disclosure and/or oxygen management and/or control within systems of the present disclosure, at least in part due to the importance of oxygen to ensure expressed polypeptides are fully functional.

What is claimed is:

1. A system of automated high-throughput screening of one or more polypeptides, the system comprising:
an interrogation chamber defining a channel having a dimension based on a size of one or more microreactors comprising the one or more polypeptides and one or more putative binding partners, the channel configured to transport the one or more microreactors;
one or more electrodes disposed at a surface of the interrogation chamber according to the dimension, the one or more electrodes to generate an electric field to hold the one or more microreactors in the channel;
a camera oriented toward a face of the interrogation chamber, the camera to capture data corresponding to the one or more microreactors in the channel; and
a memory and one or more processors configured to
determine, based on the captured data, one or more binding affinities of a polypeptide with a putative binding partner, and
generate, by a machine learning model receiving input based on the one or more binding affinities, output indicative of one or more predicted amino acid sequences of a polypeptide.

2. The system of claim 1, further comprising:
a robotic device to deposit each of one or more sample fluids comprising one or more polypeptides into one or more respective containers, wherein each respective container corresponds to one or more of the predicted amino acid sequences.

3. The system of claim 1, further comprising:
a microfluidic channel to combine one or more sample fluids comprising one or more polypeptides with one or more corresponding encapsulating fluids to form one or more microreactors in a carrier fluid.

4. The system of claim 1, wherein the camera captures first data corresponding to a first microreactor among the microreactors.

5. The system of claim 4, wherein the memory and the one or more processors are configured to:
determine, based on the captured first data, a first binding affinity of a first instance of the polypeptide in a first instance of the sample fluid comprised within the first microreactor.

6. The system of claim 4, wherein the camera captures second data corresponding to a second microreactor among the microreactors.

7. A device for containing one or more microreactors comprising:
a first transparent panel having a planar surface, the transparent panel having a rectangular shape;
a second transparent panel having the planar surface, the transparent panel having the rectangular shape; and
a gasket disposed between the first transparent panel and the second transparent panel, the first gasket having a dimension orthogonal to the planar surface that corresponds to a size of one or more microreactors.

8. The device of claim 7, wherein the gasket has a first surface in contact with a planar surface of the first transparent panel, and the gasket has a second surface facing a planar surface of the second transparent panel.

9. The device of claim 7, wherein the first gasket has a shape corresponding to the rectangular shape of the transparent panel.

10. The device of claim 7, further comprising:
a second gasket disposed between the gasket and the second transparent panel, the second gasket having a channel that corresponds to a size of one or more microreactors.

11. The device of claim 7, further comprising:
a first clamp having a planar surface in contact with the first transparent panel; and
a second clamp having a planar surface in contact with the second transparent panel.

12. The device of claim 11, wherein the first clamp is coupled with the second clamp to create a watertight fit between the first transparent panel, the second transparent panel, and the gasket.

13. An automated high-throughput method for obtaining one or more predicted amino acid sequences of a polypeptide, the method comprising:
transporting one or more microreactors comprising one or more polypeptides and one or more putative binding partners through a channel of an interrogation chamber;
capturing data corresponding to the one or more microreactors comprising the one or more polypeptides and one or more putative binding partners in the channel;
determining one or more binding affinities of the one or more polypeptides to the one or more putative binding partners based on the captured data; and
generating, by a machine learning model receiving input based on the one or more binding affinities, output indicative of one or more predicted amino acid sequences of a polypeptide.

14. The method of claim 13, further comprising:
generating, based on the one or more predicted amino acid sequences of the polypeptide, one or more DNA sequences that encode the one or more predicted amino acid sequences of the polypeptide.

15. The method of claim 13, further comprising:
correlating corresponding ones of the one or more microreactors with corresponding ones of the one or more predicted amino acid sequences of a polypeptide, based on timestamps of the captured data.

16. The method of claim 13, further comprising:
depositing, by a robotic device, each of one or more sample fluids comprising one or more polypeptides into one or more respective containers, wherein each respective container corresponds to one or more of the predicted amino acid sequences of a polypeptide.

17. The method of claim 16, wherein the depositing is performed subsequently to the generating, to iteratively identify predicted amino acid sequences with increased binding affinity to the putative binding partner.

18. The method of claim 13, wherein the one or more putative binding partners comprises an antigen or a protein of a virus.

19. The method of claim 13, further comprising:
measuring, managing, and/or controlling oxygen levels within the one or more microreactors.

20. The method of claim 13, further comprising:
selecting one or more sets of microreactors in the channel of the interrogation chamber;
monitor positions of the selected one or more sets of microreactors within the channel of the interrogation chamber over time,
wherein generating the output indicative of the one or more predicted amino acid sequences of the polypeptide comprises generating, by the machine learning model, the output indicative of the one or more predicted amino acid sequences based on the monitored positions of the selected one or more sets of microreactors.
